(19) United States
(12) Patent Application Publication
(10) Pub. No.: US 2026/0355144 A1
(43) Pub. Date: Nov. 12, 2026

(54) METAP2 INHIBITORS FOR THE TREATMENT OF PULMONARY FIBROSIS

(71) Applicant: SynDevRx , Inc., Cambridge, MA (US)

(72) Inventors: Peter CORNELIUS, Framingham, MA (US); Bradley J. CARVER, Cambridge, MA (US); Sebastien LABBE, St-Denis-De-Brompton (CA); James SHANAHAN, Cambridge, MA (US)

(21) Appl. No.: 19/102,572

(22) PCT Filed: Aug. 10, 2023
(86) PCT No.: PCT/US2023/072023

§ 371 Date: Feb. 10, 2025

Related U.S. Application Data
(60) Provisional application No. 63/396,836, filed on Aug. 10, 2022.
(60) Provisional application No. 63/405,973, filed on Sep. 13, 2022.

(51) Int. Cl. A61K 31/336; A61K 31/4418; A61K 31/496; A61K 45/06; A61K 47/58; A61K 47/65; A61K 47/68; A61P 11/00

(52) U.S. Cl. CPC A61K 31/336; A61K 31/4418; A61K 31/496; A61K 45/06; A61K 47/58; A61K 47/65; A61K 47/68037; A61K 47/6855; A61P 11/00

(57) ABSTRACT

The present disclosure provides pharmaceutical combinations comprising MetAP2 inhibitors for the treatment of interstitial lung disease, including pulmonary fibrosis.

### RELATED APPLICATIONS

This application claims priority to, and the benefit of, U.S. Provisional Application No. 63/396,836, filed Aug. 10, 2022 and U.S. Provisional Application No. 63/405,973, filed Sep. 13, 2022. The contents of each of the aforementioned patent applications are incorporated herein by reference in their entireties.

### BACKGROUND

Interstitial lung disease (ILD) describes a large group of disorders, most of which cause progressive scarring of pulmonary tissue due to fibrosis. The scarring associated with interstitial lung disease eventually affects a person's ability to breathe and get enough oxygen into their bloodstream leading to a cascade of adverse health outcomes. Interstitial lung disease can be caused by long-term exposure to hazardous materials, such as asbestos. Some types of autoimmune diseases, such as rheumatoid arthritis, also can cause interstitial lung disease. Interstitial lung disease can also be induced by the administration of specific drugs, including anti-cancer drugs (see Schwaiblmair et al. Drug induced interstitial lung disease. Open Respir Med J. 2012; 6:63-74. doi: 10.2174/1874306401206010063. Epub 2012 Jul. 27. PMID: 22896776; PMCID: PMC3415629; Camus et al. Interstitial lung disease associated with drug therapy. Br J Cancer. 2004; 91 Suppl 2 (Suppl 2): S18-S23; Conte et al. Drug-induced interstitial lung disease during cancer therapies: expert opinion on diagnosis and treatment. ESMO Open. 2022; 7 (2): 100404; the aforementioned publications are herein incorporated by reference in their entireties for all purposes). For example, Enhertu, an antibody-drug conjugate used to treat HER2-positive metastatic breast cancer, has been shown to induce ILD, including pneumonitis, in certain patients. In some cases, however, the causes remain unknown (i.e., idiopathic).

Pulmonary fibrosis, also referred to as lung fibrosis, is a complex disease that includes multiple sub-types, including idiopathic pulmonary fibrosis (IPF), which is a progressive, chronic and one of the most frequently fatal among interstitial lung diseases. Pulmonary fibrosis, including IPF, is characterized by extracellular matrix (ECM) remodeling and abnormal proliferation of fibroblasts in the pulmonary parenchyma (pulmonary foci) leading to edema and tissue scarring. Pulmonary fibrosis is thought to be the result of long-term exposure to substances harmful to alveolar epithelial cells and heredity. The pathogenesis of pulmonary fibrosis has not yet been completely resolved, but various cytokines and other molecules have been reported to play crucial roles in the progression of the disease. Activated lung epithelium might also produce mediators of fibroblast migration, proliferation, and differentiation into active myofibroblasts. These myofibroblasts secrete increased amounts of the extracellular matrix fluids and molecules which promote lung architecture remodeling.

Inflammation signaling pathways have been implicated in the pathophysiology of lung fibrosis. Acute inflammatory reactions play an important role in triggering fibrosis in the lungs, for example, in the bleomycin-induced pulmonary fibrosis model that is used to study pulmonary fibrosis in animal models. Brief exposure to bleomycin causes epithelial cell apoptosis, activating an inflammatory, wound-healing response that can lead to a temporary excess deposition of ECM components in the affected tissues. Consequently, a reduction of the inflammatory response may halt or slow the progression of tissue remodeling and allow the normal tissue architecture to be restored after injury. Fibrosis is facilitated by a number of signaling molecules such as pro-inflammatory cytokines.

Although the pathogenesis of pulmonary fibrosis remains incompletely understood, evidence suggests that several classes of proteases may play key roles in disease pathogenesis. For example, expression of matrix metalloproteinase (e.g., MMP-7 and MMP-9), as well as certain cathepsins (e.g., cathepsins B and H), are increased in lung samples from patients with lung fibrosis, and promote development of pulmonary fibrosis and/or extracellular matrix remodeling in experimental models. Another class of proteases which comprises the aminopeptidase family, has also been implicated in several disease models of fibrosis, and increased aminopeptidase activity was found in bronchoalveolar lavage fluid from patients with IPF and from patients with interstitial lung involvement from collagen vascular disease compared with normal volunteers. One member of the aminopeptidase family, is methionine aminopeptidase type 2 (MetAP2, or p67), which catalyzes the removal of the N-terminal methionine from nascent polypeptides.

There is a need in the art for compositions and methods directed to the treatment of ILDs. Additionally, there is a need in the art for composition and methods for preventing and/or mitigating treatment-induced ILDs. The present disclosure provides MetAP2 inhibitors and combinations comprising MetAP2 inhibitors for the treatment of ILDs, including pulmonary edema, pleural effusions, pulmonary fibrosis and pneumonitis, and for preventing and/or mitigating treatment-induced ILDs.

### SUMMARY

The present disclosure provides at least one MetAP2 inhibitor, or a pharmaceutically acceptable salt thereof, for use in treating pulmonary fibrosis in a subject.

The present disclosure provides methods of treating pulmonary fibrosis in a subject in need thereof, the methods comprising administering to the subject at least one therapeutically effective amount of at least one MetAP2 inhibitor, or a pharmaceutically acceptable salt thereof.

The present disclosure provides combinations comprising at least one MetAP2 inhibitor, or a pharmaceutically acceptable salt thereof, and nintedanib, or a pharmaceutically acceptable salt thereof, for use in treating pulmonary fibrosis in a subject.

The present disclosure provides methods of treating pulmonary fibrosis in a subject in need thereof, the methods comprising administering to the subject at least one therapeutically effective amount of at least one MetAP2 inhibitor, or a pharmaceutically acceptable salt thereof, and at least one therapeutically effective amount of nintedanib, or a pharmaceutically acceptable salt thereof.

The present disclosure provides MetAP2 inhibitors, or a pharmaceutically acceptable salt thereof, for use in methods of treating pulmonary fibrosis in a subject, wherein the methods further comprise administration of nintedanib, or a pharmaceutically acceptable salt thereof.

The present disclosure provides nintedanib, or a pharmaceutically acceptable salt thereof, for use in methods of treating pulmonary fibrosis in a subject, wherein the methods further comprise administration of at least one MetAP2 inhibitor or a pharmaceutically acceptable salt thereof.

The present disclosure provides combinations comprising at least one MetAP2 inhibitor, or a pharmaceutically acceptable salt thereof, and pirfenidone, or a pharmaceutically acceptable salt thereof, for use in treating pulmonary fibrosis in a subject.

The present disclosure provides methods of treating pulmonary fibrosis in a subject in need thereof, the methods comprising administering to the subject at least one therapeutically effective amount of at least one MetAP2 inhibitor, or a pharmaceutically acceptable salt thereof, and at least one therapeutically effective amount of pirfenidone, or a pharmaceutically acceptable salt thereof.

The present disclosure provides MetAP2 inhibitors, or a pharmaceutically acceptable salt thereof, for use in methods of treating pulmonary fibrosis in a subject, wherein the methods further comprise administration of pirfenidone, or a pharmaceutically acceptable salt thereof.

The present disclosure provides pirfenidone, or a pharmaceutically acceptable salt thereof, for use in methods of treating pulmonary fibrosis in a subject, wherein the methods further comprise administration of at least one MetAP2 inhibitor, or a pharmaceutically acceptable salt thereof.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a series of graphs showing the PenH value (left panel) and respiratory rate (right panel) of the mice in each treatment group on Day 6 of the study described in Example 1 of the present disclosure following randomization of the mice into the different treatment groups but before the start of treatment.

FIG. 2 is a series of graphs showing the PenH value on day 13 of the study described in Example 1 of the present disclosure (top left panel), the change in PenH value from day 6 to day 13 of the study described in Example 1 of the present disclosure (bottom left panel), the respiratory rate on day 13 of the study described in Example 1 of the present disclosure (top right panel) and the change in respiratory rate from day 6 to day 13 of the study described in Example 1 of the present disclosure (bottom rate panel) in each treatment group described in Example 1 of the present disclosure.

FIG. 3 is a series of graphs showing the PenH value on day 20 of the study described in Example 1 of the present disclosure (top left panel), the change in PenH value from day 6 to day 20 of the study described in Example 1 of the present disclosure (bottom left panel), the respiratory rate on day 20 of the study described in Example 1 of the present disclosure (top right panel) and the change in respiratory rate from day 6 to day 20 of the study described in Example 1 of the present disclosure (bottom rate panel) in each treatment group described in Example 1 of the present disclosure.

FIG. 4 is a series of graphs showing the PenH values (left panel) and respiratory rates (right panel) in each of the treatment groups over the course of the study described in Example 1 of the present disclosure.

FIG. 5A is a series of graphs showing oxygen pressure in blood (pO₂), blood bicarbonate (HCO3−) ion levels and blood lactate levels are shown (left panel to right panel, respectively) in each of the treatment groups at the conclusion of the study described in Example 1 of the present disclosure.

FIG. 5B is a graph showing arterial blood saturation (SpO₂) in each of the treatment groups at the conclusion of the study described in Example 1 of the present disclosure.

FIG. 6 is a graph showing the lung weights in each of the treatment groups at the conclusion of the study described in Example 1 of the present disclosure.

FIG. 7 is a graph showing pressure-volume curves (PV-loop) in each of the treatment groups at the conclusion of the study described in Example 1 of the present disclosure.

FIG. 8 is a graph showing the Static compliance (Cst) in each of the treatment groups at the conclusion of the study described in Example 1 of the present disclosure.

FIG. 9 is a series of graphs showing Inflation volume (tidal volume) and pulmonary capacity (total inspiratory capacity) in each of the treatment groups at the conclusion of the study described in Example 1 of the present disclosure.

FIG. 10 is a series of graphs showing Resistance of the Respiratory System (Rrs) and Tissue Damping (G) in each of the treatment groups at the conclusion of the study described in Example 1 of the present disclosure.

FIG. 11 is a series of graphs showing Elastance of the respiratory system (Ers) and pulmonary elastance (H) in each of the treatment groups at the conclusion of the study described in Example 1 of the present disclosure.

FIG. 12 is a series of graphs showing the results of automated histopathological analysis of lung samples obtained from each of the treatment groups at the conclusion of the study described in Example 1 of the present disclosure.

FIG. 13 is a series of graphs showing the body weight and change in body weight in each of the treatment groups over the course of the study described in Example 2 of the present disclosure.

FIG. 14 is a graph showing the weight of the left lung in each of the treatment groups at the conclusion of the study described in Example 2 of the present disclosure.

FIG. 15 is a graph showing the weight of the post-caval lobe weight in each of the treatment groups at the conclusion of the study described in Example 2 of the present disclosure.

FIG. 16 is a graph showing the survival of mice in each of the treatment groups over the course of the study described in Example 2 of the present disclosure.

FIGS. 17-19 is a series of graphs showing the results of automated histopathological analysis of lung samples obtained from each of the treatment groups at the conclusion of the study described in Example 2 of the present disclosure.

FIG. 17 is a graph showing the results of automated histopathological analysis to quantify tissue density in lung samples from mice from each treatment group at the conclusion of the study described in Example 2 of the present disclosure.

FIG. 18 is a graph showing the results of automated histopathological analysis to quantify fibrotic foci in lung samples from mice from each treatment group at the conclusion of the study described in Example 2 of the present disclosure.

FIG. 19 is a graph showing the results of automated histopathological analysis to quantify collagen amounts in lung samples from mice from each treatment group at the conclusion of the study described in Example 2 of the present disclosure.

FIG. 20 is a table showing the results of automated histopathological analysis to quantify the mean airspace circularity in each of the treatment groups at the conclusion of the study described in Example 1 of the present disclosure.

FIG. 21 is a table showing the results of automated histopathological analysis to quantify the mean airspace contactness in each of the treatment groups at the conclusion of the study described in Example 1 of the present disclosure.

FIG. 22 is a graph showing the results of automated histopathological analysis of lung samples obtained from each of the treatment groups at the conclusion of the study described in Example 1 representing the collagen area (COL1A1 area (%)). Values are Represented as Mean±SD, and icons represent mean value per subject.

### DETAILED DESCRIPTION

The present disclosure provides, inter alia, a method of treating interstitial lung diseases, including pulmonary fibrosis, idiopathic pulmonary fibrosis (IPF), and treatment-induced interstitial lung diseases, comprising administering to a subject in need thereof at least one therapeutically effective amount of at least one MetAP2 inhibitor of the present disclosure, or a pharmaceutically acceptable salt thereof, or comprising administering to a subject in need thereof at least one therapeutically effective amount of at least one MetAP2 inhibitor of the present disclosure, or a pharmaceutically acceptable salt thereof in combination with at least one additional therapeutic agent.

### Methods and Combinations of the Present Disclosure

The present disclosure provides methods of treating pulmonary fibrosis in a subject in need thereof, the method comprising administering to the subject in need thereof at least one therapeutically effective amount of at least one MetAP2 inhibitor of the present disclosure, or a pharmaceutically acceptable salt thereof.

The present disclosure provides a method of treating pulmonary fibrosis in a subject, the method comprising administering to the subject a MetAP2 inhibitor of the present disclosure, or a pharmaceutically acceptable salt thereof.

The present disclosure provides at least one MetAP2 inhibitor of the present disclosure, or a pharmaceutically acceptable salt thereof, for use in treating pulmonary fibrosis in a subject in need thereof.

The present disclosure provides the use of at least one MetAP2 inhibitor of the present disclosure, or a pharmaceutically acceptable salt thereof, for the manufacture of a medicament for the treatment of pulmonary fibrosis in a subject in need thereof.

The present disclosure provides a method of preventing pulmonary fibrosis in a subject in need thereof, the method comprising administering to the subject in need thereof at least one therapeutically effective amount of at least one MetAP2 inhibitor of the present disclosure, or a pharmaceutically acceptable salt thereof.

The present disclosure provides a method of preventing pulmonary fibrosis in a subject, the method comprising administering to the subject a MetAP2 inhibitor of the present disclosure, or a pharmaceutically acceptable salt thereof.

The present disclosure provides at least one MetAP2 inhibitor of the present disclosure, or a pharmaceutically acceptable salt thereof, for use in preventing pulmonary fibrosis in a subject in need thereof.

The present disclosure provides the use of at least one MetAP2 inhibitor of the present disclosure, or a pharmaceutically acceptable salt thereof, for the manufacture of a medicament for the prevention of pulmonary fibrosis in a subject in need thereof.

The present disclosure provides a combination therapy comprising at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a combination therapy comprising at least one MetAP2 inhibitor of the present disclosure, or a pharmaceutically acceptable salt thereof, and pirfenidone, or a pharmaceutically acceptable salt thereof.

In some embodiments, the combination therapy comprises a MetAP2 inhibitor of the present disclosure, or a pharmaceutically acceptable salt thereof, and pirfenidone.

In some embodiments, the combination therapy comprises a MetAP2 inhibitor of the present disclosure, or a pharmaceutically acceptable salt thereof, and nintedanib.

The present disclosure provides a method of treating pulmonary fibrosis in a subject in need thereof, the method comprising administering to the subject in need thereof at least one therapeutically effective amount of the preceding combination therapy.

The present disclosure provides a method of preventing pulmonary fibrosis in a subject in need thereof, the method comprising administering to the subject in need thereof at least one therapeutically effective amount of the preceding combination therapy.

The present disclosure provides a pharmaceutical composition comprising at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a pharmaceutical composition comprising at least one MetAP2 inhibitor of the present disclosure, or a pharmaceutically acceptable salt thereof, and pirfenidone, or a pharmaceutically acceptable salt thereof.

The present disclosure provides a method of treating pulmonary fibrosis in a subject in need thereof, the method comprising administering to the subject in need thereof at least one therapeutically effective amount of the preceding pharmaceutical composition.

The present disclosure provides a method of preventing pulmonary fibrosis in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a kit comprising least one MetAP2 inhibitor of the present disclosure, or a pharmaceutically acceptable salt thereof, and pirfenidone, or a pharmaceutically acceptable salt thereof.

The present disclosure provides a method of treating pulmonary fibrosis in a subject in need thereof, the method comprising administering to the subject in need thereof at least one therapeutically effective amount of the preceding kit.

The present disclosure provides a method of preventing pulmonary fibrosis in a subject in need thereof, the method comprising administering to the subject in need thereof at least one therapeutically effective amount of the preceding kit.

The present disclosure provides a method of treating pulmonary fibrosis in a subject in need thereof, the method comprising administering to the subject at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a method of treating pulmonary fibrosis in a subject in need thereof, the method comprising administering to the subject a MetAP2 inhibitor of the present disclosure, or a pharmaceutically acceptable salt thereof, and pirfenidone, or a pharmaceutically acceptable salt thereof.

The present disclosure provides a method of preventing pulmonary fibrosis in a subject in need thereof, the method comprising administering to the subject at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a method of preventing pulmonary fibrosis in a subject in need thereof, the method comprising administering to the subject a MetAP2 inhibitor of the present disclosure, or a pharmaceutically acceptable salt thereof, and pirfenidone, or a pharmaceutically acceptable salt thereof.

The present disclosure provides a use of at least one MetAP2 inhibitor of the present disclosure, or a pharmaceutically acceptable salt thereof, in combination with pirfenidone, or a pharmaceutically acceptable salt thereof, in the manufacture of a medicament for the treatment of pulmonary fibrosis.

The present disclosure provides a use of at least one MetAP2 inhibitor of the present disclosure, or a pharmaceutically acceptable salt thereof, in combination with pirfenidone, or a pharmaceutically acceptable salt thereof, in the manufacture of a medicament for the prevention of pulmonary fibrosis.

The present disclosure provides a use of pirfenidone, or a pharmaceutically acceptable salt thereof, in combination with at least one MetAP2 inhibitor of the present disclosure, or a pharmaceutically acceptable salt thereof, in the manufacture of a medicament for the treatment of pulmonary fibrosis.

The present disclosure provides a use of pirfenidone, or a pharmaceutically acceptable salt thereof, in combination with at least one MetAP2 inhibitor of the present disclosure, or a pharmaceutically acceptable salt thereof, in the manufacture of a medicament for the prevention of pulmonary fibrosis.

The present disclosure provides a combination of at least one MetAP2 inhibitor of the present disclosure, or a pharmaceutically acceptable salt thereof, and pirfenidone, or a pharmaceutically acceptable salt thereof, for use in the manufacture of a medicament for the treatment of pulmonary fibrosis.

The present disclosure provides a combination of at least one MetAP2 inhibitor of the present disclosure, or a pharmaceutically acceptable salt thereof, and pirfenidone, or a pharmaceutically acceptable salt thereof, for use in the manufacture of a medicament for the prevention of pulmonary fibrosis.

The present disclosure provides at least one MetAP2 inhibitor of the present disclosure, or a pharmaceutically acceptable salt thereof, for use in combination with pirfenidone, or a pharmaceutically acceptable salt thereof, in treating pulmonary fibrosis.

The present disclosure provides at least one MetAP2 inhibitor of the present disclosure, or a pharmaceutically acceptable salt thereof, for use in combination with pirfenidone, or a pharmaceutically acceptable salt thereof, in preventing pulmonary fibrosis.

The present disclosure provides pirfenidone, or a pharmaceutically acceptable salt thereof, for use in combination with at least one MetAP2 inhibitor of the present disclosure, or a pharmaceutically acceptable salt thereof, in treating pulmonary fibrosis.

The present disclosure provides pirfenidone, or a pharmaceutically acceptable salt thereof, for use in combination with at least one MetAP2 inhibitor of the present disclosure, or a pharmaceutically acceptable salt thereof, in preventing pulmonary fibrosis.

The present disclosure provides a combination of at least one MetAP2 inhibitor of the present disclosure, or a pharmaceutically acceptable salt thereof, and pirfenidone, or a pharmaceutically acceptable salt thereof, for use in treating pulmonary fibrosis.

The present disclosure provides a combination of at least one MetAP2 inhibitor of the present disclosure, or a pharmaceutically acceptable salt thereof, and pirfenidone, or a pharmaceutically acceptable salt thereof, for use in preventing pulmonary fibrosis.

The present disclosure provides a combination comprising at least one MetAP2 inhibitor of the present disclosure, or a pharmaceutically acceptable salt thereof, and pirfenidone, or a pharmaceutically acceptable salt thereof, for use in treating pulmonary fibrosis. The present disclosure provides a combination comprising at least one MetAP2 inhibitor of the present disclosure, or a pharmaceutically acceptable salt thereof, for use in treating pulmonary fibrosis, wherein the combination further comprises pirfenidone, or a pharmaceutically acceptable salt thereof. The present disclosure provides a combination comprising pirfenidone, or a pharmaceutically acceptable salt thereof, for use in treating pulmonary fibrosis, wherein the combination further comprises at least one MetAP2 inhibitor of the present disclosure, or a pharmaceutically acceptable salt thereof.

The present disclosure provides a combination comprising at least one MetAP2 inhibitor of the present disclosure, or a pharmaceutically acceptable salt thereof, and pirfenidone, or a pharmaceutically acceptable salt thereof, for use in preventing pulmonary fibrosis. The present disclosure provides a combination comprising at least one MetAP2 inhibitor of the present disclosure, or a pharmaceutically acceptable salt thereof, for use in preventing pulmonary fibrosis, wherein the combination further comprises pirfenidone, or a pharmaceutically acceptable salt thereof. The present disclosure provides a combination comprising pirfenidone, or a pharmaceutically acceptable salt thereof, for use in preventing pulmonary fibrosis, wherein the combination further comprises at least one MetAP2 inhibitor of the present disclosure, or a pharmaceutically acceptable salt thereof.

The present disclosure provides a MetAP2 inhibitor, or a pharmaceutically acceptable salt thereof, for use in a method of treating pulmonary fibrosis, wherein the method further comprises administration of pirfenidone, or a pharmaceutically acceptable salt thereof

The present disclosure provides pirfenidone, or a pharmaceutically acceptable salt thereof, for use in a method of treating pulmonary fibrosis, wherein the method further comprises administration of at least one MetAP2 inhibitor, or a pharmaceutically acceptable salt thereof.

The present disclosure provides a MetAP2 inhibitor, or a pharmaceutically acceptable salt thereof, for use in a method of preventing pulmonary fibrosis, wherein the method further comprises administration of pirfenidone, or a pharmaceutically acceptable salt thereof.

The present disclosure provides pirfenidone, or a pharmaceutically acceptable salt thereof, for use in a method of preventing pulmonary fibrosis, wherein the method further comprises administration of at least one MetAP2 inhibitor, or a pharmaceutically acceptable salt thereof.

In some aspects, a MetAP2 inhibitor, or pharmaceutically acceptable salt thereof, and pirfenidone, or pharmaceutically acceptable salt thereof, can be administered by the same administration route. In some aspects, a MetAP2 inhibitor, or pharmaceutically acceptable salt thereof, and pirfenidone, or pharmaceutically acceptable salt thereof, can be administered by different administration routes.

In some aspects, a MetAP2 inhibitor, or pharmaceutically acceptable salt thereof, and pirfenidone, or pharmaceutically acceptable salt thereof, can be administered concurrently.

In some aspects, a MetAP2 inhibitor, or pharmaceutically acceptable salt thereof, and pirfenidone, or pharmaceutically acceptable salt thereof, can be administered in temporal proximity.

In some aspects, a MetAP2 inhibitor, or pharmaceutically acceptable salt thereof, and pirfenidone, or pharmaceutically acceptable salt thereof, can be administered in any order.

The present disclosure provides a combination therapy comprising at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a combination therapy comprising at least one MetAP2 inhibitor of the present disclosure, or a pharmaceutically acceptable salt thereof, and nintedanib, or a pharmaceutically acceptable salt thereof.

The present disclosure provides a method of treating pulmonary fibrosis in a subject in need thereof, the method comprising administering to the subject in need thereof at least one therapeutically effective amount of the preceding combination therapy.

The present disclosure provides a method of preventing pulmonary fibrosis in a subject in need thereof, the method comprising administering to the subject in need thereof at least one therapeutically effective amount of the preceding combination therapy.

The present disclosure provides a pharmaceutical comprising at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a method of treating pulmonary fibrosis in a subject in need thereof, the method comprising administering to the subject in need thereof at least one therapeutically effective amount of the preceding pharmaceutical composition.

The present disclosure provides a method of preventing pulmonary fibrosis in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a kit comprising at least one MetAP2 inhibitor of the present disclosure, or a pharmaceutically acceptable salt thereof, and nintedanib, or a pharmaceutically acceptable salt thereof.

The present disclosure provides a method of treating pulmonary fibrosis in a subject in need thereof, the method comprising administering to the subject in need thereof at least one therapeutically effective amount of the preceding kit.

The present disclosure provides a method of preventing pulmonary fibrosis in a subject in need thereof, the method comprising administering to the subject in need thereof at least one therapeutically effective amount of the preceding kit.

The present disclosure provides a method of treating pulmonary fibrosis in a subject in need thereof, the method comprising administering to the subject at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a method of treating pulmonary fibrosis in a subject, the method comprising administering to the subject a MetAP2 inhibitor of the present disclosure, or a pharmaceutically acceptable salt thereof, and nintedanib, or a pharmaceutically acceptable salt thereof.

The present disclosure provides a method of preventing pulmonary fibrosis in a subject in need thereof, the method comprising administering to the subject at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a method of preventing pulmonary fibrosis in a subject, the method comprising administering to the subject a MetAP2 inhibitor of the present disclosure, or a pharmaceutically acceptable salt thereof, and nintedanib, or a pharmaceutically acceptable salt thereof.

The present disclosure provides a use of at least one MetAP2 inhibitor of the present disclosure, or a pharmaceutically acceptable salt thereof, in combination with nintedanib, or a pharmaceutically acceptable salt thereof, in the manufacture of a medicament for the treatment of pulmonary fibrosis.

The present disclosure provides a use of at least one MetAP2 inhibitor of the present disclosure, or a pharmaceutically acceptable salt thereof, in combination with nintedanib, or a pharmaceutically acceptable salt thereof, in the manufacture of a medicament for the prevention of pulmonary fibrosis.

The present disclosure provides a use of nintedanib, or a pharmaceutically acceptable salt thereof, in combination with at least one MetAP2 inhibitor of the present disclosure, or a pharmaceutically acceptable salt thereof, in the manufacture of a medicament for the treatment of pulmonary fibrosis.

The present disclosure provides a use of nintedanib, or a pharmaceutically acceptable salt thereof, in combination with at least one MetAP2 inhibitor of the present disclosure, or a pharmaceutically acceptable salt thereof, in the manufacture of a medicament for the prevention of pulmonary fibrosis.

The present disclosure provides a combination of at least one MetAP2 inhibitor of the present disclosure, or a pharmaceutically acceptable salt thereof, and nintedanib, or a pharmaceutically acceptable salt thereof, for use in the manufacture of a medicament for the treatment of pulmonary fibrosis.

The present disclosure provides a combination of at least one MetAP2 inhibitor of the present disclosure, or a pharmaceutically acceptable salt thereof, and nintedanib, or a pharmaceutically acceptable salt thereof, for use in the manufacture of a medicament for the prevention of pulmonary fibrosis.

The present disclosure provides at least one MetAP2 inhibitor of the present disclosure, or a pharmaceutically acceptable salt thereof, for use in combination with nintedanib, or a pharmaceutically acceptable salt thereof, in treating pulmonary fibrosis.

The present disclosure provides at least one MetAP2 inhibitor of the present disclosure, or a pharmaceutically acceptable salt thereof, for use in combination with nintedanib, or a pharmaceutically acceptable salt thereof, in preventing pulmonary fibrosis.

The present disclosure provides nintedanib, or a pharmaceutically acceptable salt thereof, for use in combination with at least one MetAP2 inhibitor of the present disclosure, or a pharmaceutically acceptable salt thereof, in treating pulmonary fibrosis.

The present disclosure provides nintedanib, or a pharmaceutically acceptable salt thereof, for use in combination with at least one MetAP2 inhibitor of the present disclosure, or a pharmaceutically acceptable salt thereof, in preventing pulmonary fibrosis.

The present disclosure provides a combination of at least one MetAP2 inhibitor of the present disclosure, or a pharmaceutically acceptable salt thereof, and nintedanib, or a pharmaceutically acceptable salt thereof, for use in treating pulmonary fibrosis.

The present disclosure provides a combination of at least one MetAP2 inhibitor of the present disclosure, or a pharmaceutically acceptable salt thereof, and nintedanib, or a pharmaceutically acceptable salt thereof, for use in preventing pulmonary fibrosis.

The present disclosure provides a combination comprising at least one MetAP2 inhibitor of the present disclosure, or a pharmaceutically acceptable salt thereof, and nintedanib, or a pharmaceutically acceptable salt thereof, for use in treating pulmonary fibrosis. The present disclosure provides a combination comprising at least one MetAP2 inhibitor of the present disclosure, or a pharmaceutically acceptable salt thereof, for use in treating pulmonary fibrosis, wherein the combination further comprises nintedanib, or a pharmaceutically acceptable salt thereof. The present disclosure provides a combination comprising nintedanib, or a pharmaceutically acceptable salt thereof, for use in treating pulmonary fibrosis, wherein the combination further comprises at least one MetAP2 inhibitor of the present disclosure, or a pharmaceutically acceptable salt thereof.

The present disclosure provides a combination comprising at least one MetAP2 inhibitor of the present disclosure, or a pharmaceutically acceptable salt thereof, and nintedanib, or a pharmaceutically acceptable salt thereof, for use in preventing pulmonary fibrosis. The present disclosure provides a combination comprising at least one MetAP2 inhibitor of the present disclosure, or a pharmaceutically acceptable salt thereof, for use in preventing pulmonary fibrosis, wherein the combination further comprises nintedanib, or a pharmaceutically acceptable salt thereof. The present disclosure provides a combination comprising nintedanib, or a pharmaceutically acceptable salt thereof, for use in preventing pulmonary fibrosis, wherein the combination further comprises at least one MetAP2 inhibitor of the present disclosure, or a pharmaceutically acceptable salt thereof.

The present disclosure provides a MetAP2 inhibitor, or a pharmaceutically acceptable salt thereof, for use in a method of treating pulmonary fibrosis, wherein the method further comprises administration of nintedanib, or a pharmaceutically acceptable salt thereof

The present disclosure provides nintedanib, or a pharmaceutically acceptable salt thereof, for use in a method of treating pulmonary fibrosis, wherein the method further comprises administration of at least one MetAP2 inhibitor, or a pharmaceutically acceptable salt thereof.

The present disclosure provides a MetAP2 inhibitor, or a pharmaceutically acceptable salt thereof, for use in a method of preventing pulmonary fibrosis, wherein the method further comprises administration of nintedanib, or a pharmaceutically acceptable salt thereof

The present disclosure provides nintedanib, or a pharmaceutically acceptable salt thereof, for use in a method of preventing pulmonary fibrosis, wherein the method further comprises administration of at least one MetAP2 inhibitor, or a pharmaceutically acceptable salt thereof.

The present disclosure provides methods of treating an interstitial lung disease in a subject in need thereof, the method comprising administering to the subject in need thereof at least one therapeutically effective amount of at least one MetAP2 inhibitor of the present disclosure, or a pharmaceutically acceptable salt thereof.

The present disclosure provides a method of treating an interstitial lung disease in a subject, the method comprising administering to the subject a MetAP2 inhibitor of the present disclosure, or a pharmaceutically acceptable salt thereof.

The present disclosure provides at least one MetAP2 inhibitor of the present disclosure, or a pharmaceutically acceptable salt thereof, for use in treating an interstitial lung disease in a subject in need thereof.

The present disclosure provides the use of at least one MetAP2 inhibitor of the present disclosure, or a pharmaceutically acceptable salt thereof, for the manufacture of a medicament for the treatment of an interstitial lung disease in a subject in need thereof.

The present disclosure provides a method of preventing an interstitial lung disease in a subject in need thereof, the method comprising administering to the subject in need thereof at least one therapeutically effective amount of at least one MetAP2 inhibitor of the present disclosure, or a pharmaceutically acceptable salt thereof.

The present disclosure provides a method of preventing an interstitial lung disease in a subject, the method comprising administering to the subject a MetAP2 inhibitor of the present disclosure, or a pharmaceutically acceptable salt thereof.

The present disclosure provides at least one MetAP2 inhibitor of the present disclosure, or a pharmaceutically acceptable salt thereof, for use in preventing an interstitial lung disease in a subject in need thereof.

The present disclosure provides the use of at least one MetAP2 inhibitor of the present disclosure, or a pharmaceutically acceptable salt thereof, for the manufacture of a medicament for the prevention of an interstitial lung disease in a subject in need thereof.

The present disclosure provides methods of treating a treatment-induced interstitial lung disease in a subject in need thereof, the method comprising administering to the subject in need thereof at least one therapeutically effective amount of at least one MetAP2 inhibitor of the present disclosure, or a pharmaceutically acceptable salt thereof.

The present disclosure provides a method of treating a treatment-induced interstitial lung disease in a subject, the method comprising administering to the subject a MetAP2 inhibitor of the present disclosure, or a pharmaceutically acceptable salt thereof.

The present disclosure provides at least one MetAP2 inhibitor of the present disclosure, or a pharmaceutically acceptable salt thereof, for use in treating a treatment-induced interstitial lung disease in a subject in need thereof.

The present disclosure provides the use of at least one MetAP2 inhibitor of the present disclosure, or a pharmaceutically acceptable salt thereof, for the manufacture of a medicament for the treatment of a treatment-induced interstitial lung disease in a subject in need thereof.

The present disclosure provides a method of preventing a treatment-induced interstitial lung disease in a subject in need thereof, the method comprising administering to the subject in need thereof at least one therapeutically effective amount of at least one MetAP2 inhibitor of the present disclosure, or a pharmaceutically acceptable salt thereof.

The present disclosure provides a method of preventing a treatment-induced interstitial lung disease in a subject, the method comprising administering to the subject a MetAP2 inhibitor of the present disclosure, or a pharmaceutically acceptable salt thereof.

The present disclosure provides at least one MetAP2 inhibitor of the present disclosure, or a pharmaceutically acceptable salt thereof, for use in preventing a treatment-induced interstitial lung disease in a subject in need thereof.

The present disclosure provides the use of at least one MetAP2 inhibitor of the present disclosure, or a pharmaceutically acceptable salt thereof, for the manufacture of a medicament for the prevention of a treatment-induced interstitial lung disease in a subject in need thereof.

The present disclosure provides methods of preventing and/or mitigating a treatment-induced interstitial lung disease in a subject in need thereof, the method comprising administering to the subject in need thereof at least one therapeutically effective amount of at least one MetAP2 inhibitor of the present disclosure, or a pharmaceutically acceptable salt thereof.

The present disclosure provides a method of preventing and/or mitigating a treatment-induced interstitial lung disease in a subject, the method comprising administering to the subject a MetAP2 inhibitor of the present disclosure, or a pharmaceutically acceptable salt thereof.

The present disclosure provides at least one MetAP2 inhibitor of the present disclosure, or a pharmaceutically acceptable salt thereof, for use in preventing and/or mitigating a treatment-induced interstitial lung disease in a subject in need thereof.

The present disclosure provides the use of at least one MetAP2 inhibitor of the present disclosure, or a pharmaceutically acceptable salt thereof, for the manufacture of a medicament for the prevention and/or mitigation of a treatment-induced interstitial lung disease in a subject in need thereof.

The present disclosure provides a combination therapy comprising at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a method of treating an interstitial lung disease in a subject in need thereof, the method comprising administering to the subject in need thereof at least one therapeutically effective amount of the preceding combination therapy.

The present disclosure provides a method of preventing an interstitial lung disease in a subject in need thereof, the method comprising administering to the subject in need thereof at least one therapeutically effective amount of the preceding combination therapy.

The present disclosure provides a pharmaceutical comprising at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a method of treating an interstitial lung disease in a subject in need thereof, the method comprising administering to the subject in need thereof at least one therapeutically effective amount of the preceding pharmaceutical composition.

The present disclosure provides a method of preventing an interstitial lung disease in a subject in need thereof, the method comprising administering to the subject in need thereof at least one therapeutically effective amount of the preceding pharmaceutical composition.

The present disclosure provides a method of treating an interstitial lung disease in a subject in need thereof, the method comprising administering to the subject in need thereof at least one therapeutically effective amount of the preceding kit.

The present disclosure provides a method of preventing an interstitial lung disease in a subject in need thereof, the method comprising administering to the subject in need thereof at least one therapeutically effective amount of the preceding kit.

The present disclosure provides a method of treating an interstitial lung disease in a subject in need thereof, the method comprising administering to the subject at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a method of treating an interstitial lung disease in a subject, the method comprising administering to the subject a MetAP2 inhibitor of the present disclosure, or a pharmaceutically acceptable salt thereof, and apirfenidone, or a pharmaceutically acceptable salt thereof.

The present disclosure provides a method of preventing an interstitial lung disease in a subject in need thereof, the method comprising administering to the subject at least one therapeutically effective amount of at least one MetAP2 inhibitor of the present disclosure, or a pharmaceutically acceptable salt thereof, and at least one therapeutically effective amount of pirfenidone, or a pharmaceutically acceptable salt thereof.

The present disclosure provides a method of preventing an interstitial lung disease in a subject, the method comprising administering to the subject a MetAP2 inhibitor of the present disclosure, or a pharmaceutically acceptable salt thereof, and apirfenidone, or a pharmaceutically acceptable salt thereof.

The present disclosure provides a use of at least one MetAP2 inhibitor of the present disclosure, or a pharmaceutically acceptable salt thereof, in combination with pirfenidone, or a pharmaceutically acceptable salt thereof, in the manufacture of a medicament for the treatment of an interstitial lung disease.

The present disclosure provides a use of at least one MetAP2 inhibitor of the present disclosure, or a pharmaceutically acceptable salt thereof, in combination with pirfenidone, or a pharmaceutically acceptable salt thereof, in the manufacture of a medicament for the prevention of an interstitial lung disease.

The present disclosure provides a use of pirfenidone, or a pharmaceutically acceptable salt thereof, in combination with at least one MetAP2 inhibitor of the present disclosure, or a pharmaceutically acceptable salt thereof, in the manufacture of a medicament for the treatment of an interstitial lung disease.

The present disclosure provides a use of pirfenidone, or a pharmaceutically acceptable salt thereof, in combination with at least one MetAP2 inhibitor of the present disclosure, or a pharmaceutically acceptable salt thereof, in the manufacture of a medicament for the prevention of an interstitial lung disease.

The present disclosure provides a combination of at least one MetAP2 inhibitor of the present disclosure, or a pharmaceutically acceptable salt thereof, and pirfenidone, or a pharmaceutically acceptable salt thereof, for use in the manufacture of a medicament for the treatment of an interstitial lung disease.

The present disclosure provides a combination of at least one MetAP2 inhibitor of the present disclosure, or a pharmaceutically acceptable salt thereof, and pirfenidone, or a pharmaceutically acceptable salt thereof, for use in the manufacture of a medicament for the prevention of an interstitial lung disease.

The present disclosure provides at least one MetAP2 inhibitor of the present disclosure, or a pharmaceutically acceptable salt thereof, for use in combination with pirfenidone, or a pharmaceutically acceptable salt thereof, in treating an interstitial lung disease.

The present disclosure provides at least one MetAP2 inhibitor of the present disclosure, or a pharmaceutically acceptable salt thereof, for use in combination with pirfenidone, or a pharmaceutically acceptable salt thereof, in preventing an interstitial lung disease.

The present disclosure provides pirfenidone, or a pharmaceutically acceptable salt thereof, for use in combination with at least one MetAP2 inhibitor of the present disclosure, or a pharmaceutically acceptable salt thereof, in treating an interstitial lung disease.

The present disclosure provides pirfenidone, or a pharmaceutically acceptable salt thereof, for use in combination with at least one MetAP2 inhibitor of the present disclosure, or a pharmaceutically acceptable salt thereof, in preventing an interstitial lung disease.

The present disclosure provides a combination of at least one MetAP2 inhibitor of the present disclosure, or a pharmaceutically acceptable salt thereof, and pirfenidone, or a pharmaceutically acceptable salt thereof, for use in treating an interstitial lung disease.

The present disclosure provides a combination of at least one MetAP2 inhibitor of the present disclosure, or a pharmaceutically acceptable salt thereof, and pirfenidone, or a pharmaceutically acceptable salt thereof, for use in preventing an interstitial lung disease.

The present disclosure provides a combination comprising at least one MetAP2 inhibitor of the present disclosure, or a pharmaceutically acceptable salt thereof, and pirfenidone, or a pharmaceutically acceptable salt thereof, for use in treating an interstitial lung disease. The present disclosure provides a combination comprising at least one MetAP2 inhibitor of the present disclosure, or a pharmaceutically acceptable salt thereof, for use in treating an interstitial lung disease, wherein the combination further comprises pirfenidone, or a pharmaceutically acceptable salt thereof. The present disclosure provides a combination comprising pirfenidone, or a pharmaceutically acceptable salt thereof, for use in treating an interstitial lung disease, wherein the combination further comprises at least one MetAP2 inhibitor of the present disclosure, or a pharmaceutically acceptable salt thereof.

The present disclosure provides a combination comprising at least one MetAP2 inhibitor of the present disclosure, or a pharmaceutically acceptable salt thereof, and pirfenidone, or a pharmaceutically acceptable salt thereof, for use in preventing an interstitial lung disease. The present disclosure provides a combination comprising at least one MetAP2 inhibitor of the present disclosure, or a pharmaceutically acceptable salt thereof, for use in preventing an interstitial lung disease, wherein the combination further comprises pirfenidone, or a pharmaceutically acceptable salt thereof. The present disclosure provides a combination comprising pirfenidone, or a pharmaceutically acceptable salt thereof, for use in preventing an interstitial lung disease, wherein the combination further comprises at least one MetAP2 inhibitor of the present disclosure, or a pharmaceutically acceptable salt thereof.

The present disclosure provides a MetAP2 inhibitor, or a pharmaceutically acceptable salt thereof, for use in a method of treating an interstitial lung disease, wherein the method further comprises administration of pirfenidone, or a pharmaceutically acceptable salt thereof

The present disclosure provides pirfenidone, or a pharmaceutically acceptable salt thereof, for use in a method of treating an interstitial lung disease, wherein the method further comprises administration of at least one MetAP2 inhibitor, or a pharmaceutically acceptable salt thereof.

The present disclosure provides a MetAP2 inhibitor, or a pharmaceutically acceptable salt thereof, for use in a method of preventing an interstitial lung disease, wherein the method further comprises administration of pirfenidone, or a pharmaceutically acceptable salt thereof

The present disclosure provides pirfenidone, or a pharmaceutically acceptable salt thereof, for use in a method of preventing an interstitial lung disease, wherein the method further comprises administration of at least one MetAP2 inhibitor, or a pharmaceutically acceptable salt thereof.

In some aspects, a MetAP2 inhibitor, or pharmaceutically acceptable salt thereof, and pirfenidone, or pharmaceutically acceptable salt thereof, can be administered by the same administration route. In some aspects, a MetAP2 inhibitor, or pharmaceutically acceptable salt thereof, and pirfenidone, or pharmaceutically acceptable salt thereof, can be administered by different administration routes.

In some aspects, a MetAP2 inhibitor, or pharmaceutically acceptable salt thereof, and pirfenidone, or pharmaceutically acceptable salt thereof, can be administered concurrently.

In some aspects, a MetAP2 inhibitor, or pharmaceutically acceptable salt thereof, and pirfenidone, or pharmaceutically acceptable salt thereof, can be administered in temporal proximity.

In some aspects, a MetAP2 inhibitor, or pharmaceutically acceptable salt thereof, and pirfenidone, or pharmaceutically acceptable salt thereof, can be administered in any order.

The present disclosure provides a combination therapy comprising at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a method of treating an interstitial lung disease in a subject in need thereof, the method comprising administering to the subject in need thereof at least one therapeutically effective amount of the preceding combination therapy.

The present disclosure provides a method of treating an interstitial lung disease in a subject, the method comprising administering to the subject the combination therapy.

The present disclosure provides a method of preventing an interstitial lung disease in a subject in need thereof, the method comprising administering to the subject in need thereof at least one therapeutically effective amount of the preceding combination therapy.

The present disclosure provides a pharmaceutical composition comprising at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a pharmaceutical composition comprising at least one MetAP2 inhibitor of the present disclosure, or a pharmaceutically acceptable salt thereof, and nintedanib, or a pharmaceutically acceptable salt thereof.

The present disclosure provides a method of treating an interstitial lung disease in a subject in need thereof, the method comprising administering to the subject in need thereof at least one therapeutically effective amount of the preceding pharmaceutical composition.

The present disclosure provides a method of preventing an interstitial lung disease in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a method of treating an interstitial lung disease in a subject in need thereof, the method comprising administering to the subject in need thereof at least one therapeutically effective amount of the preceding kit.

The present disclosure provides a method of preventing an interstitial lung disease in a subject in need thereof, the method comprising administering to the subject in need thereof at least one therapeutically effective amount of the preceding kit.

The present disclosure provides a method of treating an interstitial lung disease in a subject in need thereof, the method comprising administering to the subject at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a method of treating an interstitial lung disease in a subject the method comprising administering to the subject a MetAP2 inhibitor of the present disclosure, or a pharmaceutically acceptable salt thereof, and nintedanib, or a pharmaceutically acceptable salt thereof.

The present disclosure provides a method of preventing an interstitial lung disease in a subject in need thereof, the method comprising administering to the subject at least one therapeutically effective amount of at least one MetAP2 inhibitor of the present disclosure, or a pharmaceutically acceptable salt thereof, and at least one therapeutically effective amount of nintedanib, or a pharmaceutically acceptable salt thereof.

The present disclosure provides a method of preventing an interstitial lung disease in a subject the method comprising administering to the subject a MetAP2 inhibitor of the present disclosure, or a pharmaceutically acceptable salt thereof, and nintedanib, or a pharmaceutically acceptable salt thereof.

The present disclosure provides a use of at least one MetAP2 inhibitor of the present disclosure, or a pharmaceutically acceptable salt thereof, in combination with nintedanib, or a pharmaceutically acceptable salt thereof, in the manufacture of a medicament for the treatment of an interstitial lung disease.

The present disclosure provides a use of at least one MetAP2 inhibitor of the present disclosure, or a pharmaceutically acceptable salt thereof, in combination with nintedanib, or a pharmaceutically acceptable salt thereof, in the manufacture of a medicament for the prevention of an interstitial lung disease.

The present disclosure provides a use of nintedanib, or a pharmaceutically acceptable salt thereof, in combination with at least one MetAP2 inhibitor of the present disclosure, or a pharmaceutically acceptable salt thereof, in the manufacture of a medicament for the treatment of an interstitial lung disease.

The present disclosure provides a use of nintedanib, or a pharmaceutically acceptable salt thereof, in combination with at least one MetAP2 inhibitor of the present disclosure, or a pharmaceutically acceptable salt thereof, in the manufacture of a medicament for the prevention of an interstitial lung disease.

The present disclosure provides a combination of at least one MetAP2 inhibitor of the present disclosure, or a pharmaceutically acceptable salt thereof, and nintedanib, or a pharmaceutically acceptable salt thereof, for use in the manufacture of a medicament for the treatment of an interstitial lung disease.

The present disclosure provides a combination of at least one MetAP2 inhibitor of the present disclosure, or a pharmaceutically acceptable salt thereof, and nintedanib, or a pharmaceutically acceptable salt thereof, for use in the manufacture of a medicament for the prevention of an interstitial lung disease.

The present disclosure provides at least one MetAP2 inhibitor of the present disclosure, or a pharmaceutically acceptable salt thereof, for use in combination with nintedanib, or a pharmaceutically acceptable salt thereof, in treating an interstitial lung disease.

The present disclosure provides at least one MetAP2 inhibitor of the present disclosure, or a pharmaceutically acceptable salt thereof, for use in combination with nintedanib, or a pharmaceutically acceptable salt thereof, in preventing an interstitial lung disease.

The present disclosure provides nintedanib, or a pharmaceutically acceptable salt thereof, for use in combination with at least one MetAP2 inhibitor of the present disclosure, or a pharmaceutically acceptable salt thereof, in treating an interstitial lung disease.

The present disclosure provides nintedanib, or a pharmaceutically acceptable salt thereof, for use in combination with at least one MetAP2 inhibitor of the present disclosure, or a pharmaceutically acceptable salt thereof, in preventing an interstitial lung disease.

The present disclosure provides a combination of at least one MetAP2 inhibitor of the present disclosure, or a pharmaceutically acceptable salt thereof, and nintedanib, or a pharmaceutically acceptable salt thereof, for use in treating an interstitial lung disease.

The present disclosure provides a combination of at least one MetAP2 inhibitor of the present disclosure, or a pharmaceutically acceptable salt thereof, and nintedanib, or a pharmaceutically acceptable salt thereof, for use in preventing an interstitial lung disease.

The present disclosure provides a combination comprising at least one MetAP2 inhibitor of the present disclosure, or a pharmaceutically acceptable salt thereof, and nintedanib, or a pharmaceutically acceptable salt thereof, for use in treating an interstitial lung disease. The present disclosure provides a combination comprising at least one MetAP2 inhibitor of the present disclosure, or a pharmaceutically acceptable salt thereof, for use in treating an interstitial lung disease, wherein the combination further comprises nintedanib, or a pharmaceutically acceptable salt thereof. The present disclosure provides a combination comprising nintedanib, or a pharmaceutically acceptable salt thereof, for use in treating an interstitial lung disease, wherein the combination further comprises at least one MetAP2 inhibitor of the present disclosure, or a pharmaceutically acceptable salt thereof.

The present disclosure provides a combination comprising at least one MetAP2 inhibitor of the present disclosure, or a pharmaceutically acceptable salt thereof, and nintedanib, or a pharmaceutically acceptable salt thereof, for use in preventing an interstitial lung disease. The present disclosure provides a combination comprising at least one MetAP2 inhibitor of the present disclosure, or a pharmaceutically acceptable salt thereof, for use in preventing an interstitial lung disease, wherein the combination further comprises nintedanib, or a pharmaceutically acceptable salt thereof. The present disclosure provides a combination comprising nintedanib, or a pharmaceutically acceptable salt thereof, for use in preventing an interstitial lung disease, wherein the combination further comprises at least one MetAP2 inhibitor of the present disclosure, or a pharmaceutically acceptable salt thereof.

The present disclosure provides a MetAP2 inhibitor, or a pharmaceutically acceptable salt thereof, for use in a method of treating an interstitial lung disease, wherein the method further comprises administration of nintedanib, or a pharmaceutically acceptable salt thereof

The present disclosure provides nintedanib, or a pharmaceutically acceptable salt thereof, for use in a method of treating an interstitial lung disease, wherein the method further comprises administration of at least one MetAP2 inhibitor, or a pharmaceutically acceptable salt thereof.

The present disclosure provides a MetAP2 inhibitor, or a pharmaceutically acceptable salt thereof, for use in a method of preventing an interstitial lung disease, wherein the method further comprises administration of nintedanib, or a pharmaceutically acceptable salt thereof

The present disclosure provides nintedanib, or a pharmaceutically acceptable salt thereof, for use in a method of preventing an interstitial lung disease, wherein the method further comprises administration of at least one MetAP2 inhibitor, or a pharmaceutically acceptable salt thereof.

In some aspects, a MetAP2 inhibitor, or pharmaceutically acceptable salt thereof, and nintedanib, or pharmaceutically acceptable salt thereof, can be administered by the same administration route. In some aspects, a MetAP2 inhibitor, or pharmaceutically acceptable salt thereof, and nintedanib, or pharmaceutically acceptable salt thereof, can be administered by different administration routes.

In some aspects, a MetAP2 inhibitor, or pharmaceutically acceptable salt thereof, and nintedanib, or pharmaceutically acceptable salt thereof, can be administered concurrently.

In some aspects, a MetAP2 inhibitor, or pharmaceutically acceptable salt thereof, and nintedanib, or pharmaceutically acceptable salt thereof, can be administered in temporal proximity.

In some aspects, a MetAP2 inhibitor, or pharmaceutically acceptable salt thereof, and nintedanib, or pharmaceutically acceptable salt thereof, can be administered in any order.

The present disclosure provides a combination therapy comprising at least one therapeutically effective amount of at least one MetAP2 inhibitor of the present disclosure, or a pharmaceutically acceptable salt thereof, and at least one therapeutically effective amount of at least one anti-cancer agent, or a pharmaceutically acceptable salt thereof.

The present disclosure provides a method of treating a cancer in a subject in need thereof, the method comprising administering to the subject in need thereof at least one therapeutically effective amount of the preceding combination therapy.

The present disclosure provides a method of treating a cancer in a subject, the method comprising administering to the subject a combination therapy of the present disclosure.

The present disclosure provides a method of preventing a cancer in a subject in need thereof, the method comprising administering to the subject in need thereof at least one therapeutically effective amount of the preceding combination therapy.

The present disclosure provides a method of preventing a cancer in a subject, the method comprising administering to the subject a combination therapy of the present disclosure.

The present disclosure provides a pharmaceutical composition comprising at least one therapeutically effective amount of at least one MetAP2 inhibitor of the present disclosure, or a pharmaceutically acceptable salt thereof, and at least one therapeutically effective amount of at least one anti-cancer agent, or a pharmaceutically acceptable salt thereof.

The present disclosure provides a method of treating a cancer in a subject in need thereof, the method comprising administering to the subject in need thereof at least one therapeutically effective amount of the preceding pharmaceutical composition.

The present disclosure provides a method of preventing a cancer in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof, and at least one therapeutically effective amount of at least one anti-cancer agent, or a pharmaceutically acceptable salt thereof.

The present disclosure provides a method of treating a cancer in a subject in need thereof, the method comprising administering to the subject in need thereof at least one therapeutically effective amount of the preceding kit.

The present disclosure provides a method of preventing a cancer in a subject in need thereof, the method comprising administering to the subject in need thereof at least one therapeutically effective amount of the preceding kit.

The present disclosure provides a method of treating a cancer in a subject in need thereof, the method comprising administering to the subject at least one therapeutically effective amount of at least one MetAP2 inhibitor of the present disclosure, or a pharmaceutically acceptable salt thereof, and at least one therapeutically effective amount of at least one anti-cancer agent, or a pharmaceutically acceptable salt thereof.

The present disclosure provides a method of treating a cancer, the method comprising administering to the subject a MetAP2 inhibitor of the present disclosure, or a pharmaceutically acceptable salt thereof, and an anti-cancer agent, or a pharmaceutically acceptable salt thereof.

The present disclosure provides a method of preventing a cancer in a subject in need thereof, the method comprising administering to the subject at least one therapeutically effective amount of at least one MetAP2 inhibitor of the present disclosure, or a pharmaceutically acceptable salt thereof, and at least one therapeutically effective amount of at least one anti-cancer agent, or a pharmaceutically acceptable salt thereof.

The present disclosure provides a method of preventing a cancer, the method comprising administering to the subject a MetAP2 inhibitor of the present disclosure, or a pharmaceutically acceptable salt thereof, and an anti-cancer agent, or a pharmaceutically acceptable salt thereof.

The present disclosure provides a use of at least one MetAP2 inhibitor of the present disclosure, or a pharmaceutically acceptable salt thereof, in combination with at least one anti-cancer agent, or a pharmaceutically acceptable salt thereof, in the manufacture of a medicament for the treatment of a cancer.

The present disclosure provides a use of at least one MetAP2 inhibitor of the present disclosure, or a pharmaceutically acceptable salt thereof, in combination with at least one anti-cancer agent, or a pharmaceutically acceptable salt thereof, in the manufacture of a medicament for the prevention of a cancer.

The present disclosure provides a use of at least one anti-cancer agent, or a pharmaceutically acceptable salt thereof, in combination with at least one MetAP2 inhibitor of the present disclosure, or a pharmaceutically acceptable salt thereof, in the manufacture of a medicament for the treatment of a cancer.

The present disclosure provides a use of at least one anti-cancer agent, or a pharmaceutically acceptable salt thereof, in combination with at least one MetAP2 inhibitor of the present disclosure, or a pharmaceutically acceptable salt thereof, in the manufacture of a medicament for the prevention of a cancer.

The present disclosure provides a combination of at least one MetAP2 inhibitor of the present disclosure, or a pharmaceutically acceptable salt thereof, and at least one anti-cancer agent, or a pharmaceutically acceptable salt thereof, for use in the manufacture of a medicament for the treatment of a cancer.

The present disclosure provides a combination of at least one MetAP2 inhibitor of the present disclosure, or a pharmaceutically acceptable salt thereof, and at least one anti-cancer agent, or a pharmaceutically acceptable salt thereof, for use in the manufacture of a medicament for the prevention of a cancer.

The present disclosure provides at least one MetAP2 inhibitor of the present disclosure, or a pharmaceutically acceptable salt thereof, for use in combination with at least one anti-cancer agent, or a pharmaceutically acceptable salt thereof, in treating a cancer.

The present disclosure provides at least one MetAP2 inhibitor of the present disclosure, or a pharmaceutically acceptable salt thereof, for use in combination with at least one anti-cancer agent, or a pharmaceutically acceptable salt thereof, in preventing a cancer.

The present disclosure provides at least one anti-cancer agent, or a pharmaceutically acceptable salt thereof, for use in combination with at least one MetAP2 inhibitor of the present disclosure, or a pharmaceutically acceptable salt thereof, in treating a cancer.

The present disclosure provides at least one anti-cancer agent, or a pharmaceutically acceptable salt thereof, for use in combination with at least one MetAP2 inhibitor of the present disclosure, or a pharmaceutically acceptable salt thereof, in preventing a cancer.

The present disclosure provides a combination of at least one MetAP2 inhibitor of the present disclosure, or a pharmaceutically acceptable salt thereof, and at least one anti-cancer agent, or a pharmaceutically acceptable salt thereof, for use in treating a cancer.

The present disclosure provides a combination of at least one MetAP2 inhibitor of the present disclosure, or a pharmaceutically acceptable salt thereof, and at least one anti-cancer agent, or a pharmaceutically acceptable salt thereof, for use in preventing a cancer.

The present disclosure provides a combination comprising at least one MetAP2 inhibitor of the present disclosure, or a pharmaceutically acceptable salt thereof, and at least one anti-cancer agent, or a pharmaceutically acceptable salt thereof, for use in treating a cancer. The present disclosure provides a combination comprising at least one MetAP2 inhibitor of the present disclosure, or a pharmaceutically acceptable salt thereof, for use in treating a cancer, wherein the combination further comprises at least one anti-cancer agent, or a pharmaceutically acceptable salt thereof. The present disclosure provides a combination comprising at least one anti-cancer agent, or a pharmaceutically acceptable salt thereof, for use in treating a cancer, wherein the combination further comprises at least one MetAP2 inhibitor of the present disclosure, or a pharmaceutically acceptable salt thereof.

The present disclosure provides a combination comprising at least one MetAP2 inhibitor of the present disclosure, or a pharmaceutically acceptable salt thereof, and at least one anti-cancer agent, or a pharmaceutically acceptable salt thereof, for use in preventing a cancer. The present disclosure provides a combination comprising at least one MetAP2 inhibitor of the present disclosure, or a pharmaceutically acceptable salt thereof, for use in preventing a cancer, wherein the combination further comprises at least one anti-cancer agent, or a pharmaceutically acceptable salt thereof. The present disclosure provides a combination comprising at least one anti-cancer agent, or a pharmaceutically acceptable salt thereof, for use in preventing a cancer, wherein the combination further comprises at least one MetAP2 inhibitor of the present disclosure, or a pharmaceutically acceptable salt thereof.

The present disclosure provides a MetAP2 inhibitor, or a pharmaceutically acceptable salt thereof, for use in a method of treating a cancer, wherein the method further comprises administration of at least one anti-cancer agent, or a pharmaceutically acceptable salt thereof

The present disclosure provides at least one anti-cancer agent, or a pharmaceutically acceptable salt thereof, for use in a method of treating a cancer, wherein the method further comprises administration of at least one MetAP2 inhibitor, or a pharmaceutically acceptable salt thereof.

The present disclosure provides a MetAP2 inhibitor, or a pharmaceutically acceptable salt thereof, for use in a method of preventing a cancer, wherein the method further comprises administration of at least one anti-cancer agent, or a pharmaceutically acceptable salt thereof

The present disclosure provides at least one anti-cancer agent, or a pharmaceutically acceptable salt thereof, for use in a method of preventing a cancer, wherein the method further comprises administration of at least one MetAP2 inhibitor, or a pharmaceutically acceptable salt thereof.

In some aspects, a MetAP2 inhibitor, or pharmaceutically acceptable salt thereof, and at least one anti-cancer agent, or pharmaceutically acceptable salt thereof, can be administered by the same administration route. In some aspects, a MetAP2 inhibitor, or pharmaceutically acceptable salt thereof, and at least one anti-cancer agent, or pharmaceutically acceptable salt thereof, can be administered by different administration routes.

In some aspects, a MetAP2 inhibitor, or pharmaceutically acceptable salt thereof, and at least one anti-cancer agent, or pharmaceutically acceptable salt thereof, can be administered concurrently.

In some aspects, a MetAP2 inhibitor, or pharmaceutically acceptable salt thereof, and at least one anti-cancer agent, or pharmaceutically acceptable salt thereof, can be administered in temporal proximity.

In some aspects, a MetAP2 inhibitor, or pharmaceutically acceptable salt thereof, and at least one anti-cancer agent, or pharmaceutically acceptable salt thereof, can be administered in any order.

In some aspects, the administration of a MetAP2 inhibitor of the present disclosure, or pharmaceutically acceptable salt thereof, alone or in combination with nintedanib and/or an anti-cancer agent, or pharmaceutically acceptable salt thereof, to the subject in need thereof results in reduced pulmonary congestion, reduced pulmonary inflammation, reduced respirator rates, increased SpO₂, increased pO₂ levels, decreased blood lactate levels, increased blood bicarbonate ion levels, decreased lung weight, reduced tissue resistance of the respiratory system, reduced tissue damping, reduced elastance of the respiratory system, reduced pulmonary elastance, reduced parenchyma density, reduced pulmonary foci, reduced collagen content, reduced collagen deposition, and/or reduced tissue density in the subject, as compared to a subject who was not administered a MetAP2 inhibitor of the present disclosure, pharmaceutically acceptable salt thereof, alone or in combination with nintedanib and/or an anti-cancer agent, pharmaceutically acceptable salt thereof.

In some aspects, the administration of a MetAP2 inhibitor of the present disclosure, or pharmaceutically acceptable salt thereof, results in reduced pulmonary congestion, reduced pulmonary inflammation, reduced respirator rates, increased SpO₂, increased pO₂ levels, decreased blood lactate levels, increased blood bicarbonate ion levels, decreased lung weight, reduced tissue resistance of the respiratory system, reduced tissue damping, reduced elastance of the respiratory system, reduced pulmonary elastance, reduced parenchyma density, reduced pulmonary foci, reduced collagen content, reduced collagen deposition, and/or reduced tissue density in the subject, as compared to a subject who was not administered a MetAP2 inhibitor of the present disclosure, pharmaceutically acceptable salt thereof.

In some aspects, the administration of a MetAP2 inhibitor of the present disclosure, or pharmaceutically acceptable salt thereof, and nintedanib, or a pharmaceutically acceptable salt thereof, results in reduced pulmonary congestion, reduced pulmonary inflammation, reduced respirator rates, increased SpO₂, increased pO₂ levels, decreased blood lactate levels, increased blood bicarbonate ion levels, decreased lung weight, reduced tissue resistance of the respiratory system, reduced tissue damping, reduced elastance of the respiratory system, reduced pulmonary elastance, reduced parenchyma density, reduced pulmonary foci, reduced collagen content, reduced collagen deposition, and/or reduced tissue density in the subject, as compared to a subject who was not administered a MetAP2 inhibitor of the present disclosure, pharmaceutically acceptable salt thereof, and nintedanib, or a pharmaceutically acceptable salt thereof.

In some aspects, the administration of a MetAP2 inhibitor of the present disclosure, or pharmaceutically acceptable salt thereof, and pirfenidone, or a pharmaceutically acceptable salt thereof, results in reduced pulmonary congestion, reduced pulmonary inflammation, reduced respirator rates, increased SpO₂, increased pO₂ levels, decreased blood lactate levels, increased blood bicarbonate ion levels, decreased lung weight, reduced tissue resistance of the respiratory system, reduced tissue damping, reduced elastance of the respiratory system, reduced pulmonary elastance, reduced parenchyma density, reduced pulmonary foci, reduced collagen content, reduced collagen deposition, and/or reduced tissue density in the subject, as compared to a subject who was not administered a MetAP2 inhibitor of the present disclosure, pharmaceutically acceptable salt thereof, and pirfenidone, or a pharmaceutically acceptable salt thereof.

In some aspects, the administration of a MetAP2 inhibitor of the present disclosure, or pharmaceutically acceptable salt thereof, and an anti-cancer agent, or a pharmaceutically acceptable salt thereof, results in reduced pulmonary congestion, reduced pulmonary inflammation, reduced respirator rates, increased SpO₂, increased pO₂ levels, decreased blood lactate levels, increased blood bicarbonate ion levels, decreased lung weight, reduced tissue resistance of the respiratory system, reduced tissue damping, reduced elastance of the respiratory system, reduced pulmonary elastance, reduced parenchyma density, reduced pulmonary foci, reduced collagen content, reduced collagen deposition, and/or reduced tissue density in the subject, as compared to a subject who was not administered a MetAP2 inhibitor of the present disclosure, pharmaceutically acceptable salt thereof, and an anti-cancer agent, or a pharmaceutically acceptable salt thereof.

In some embodiments, bleomycin (BLM) was administered intratracheally. In some embodiments, the exposure to bleomycin results in an inflammatory response and increase epithelial apoptosis. In some embodiments, this inflammatory response and epithelial apoptosis occurs within the first 7 days after exposure. In some embodiments, the exposure to bleomycin causes a condition resembling acute lung injury (ALI). In some embodiments, a fibrotic stage begins 7 days after exposure to BLM. In some embodiments, the fibrotic stage persists for 3-4 weeks post BLM exposure.

In some embodiments, the treatment with bleomycin results in pulmonary function reduction. In some embodiments, the pulmonary function reduction is indicated by an increase in pulmonary congestion, an increase in pulmonary inflammation, an increase in respiratory rates, a decrease in SpO₂ values, an increase in blood lactate levels, a decrease in blood bicarbonate ion levels, an increase in edema, an increase in vascular leakiness and extracellular matrix deposition, an increase in lung tissue resistance and stiffness, an increase parenchyma density, an increase in pulmonary foci, and an increase in the collagen content of the lung.

In some embodiments, the administration of a MetAP2 inhibitor of the present disclosure, or pharmaceutically acceptable salt thereof, results in an increase in pulmonary function as indicated by a decrease of pulmonary congestion, a decrease in pulmonary inflammation, an increase in blood bicarbonate ion levels, a decrease in edema, a decrease in vascular leakiness and extracellular matrix deposition, and an increase in SpO₂ values, a decrease in blood lactate levels, a decrease in lung tissue resistance and stiffness, a decrease in parenchyma density, a decrease in pulmonary foci, a decrease in collagen content of the lung, and a decrease in respiratory rates as compared to a subject who was not administered a MetAP2 inhibitor of the present disclosure, pharmaceutically acceptable salt thereof. In some embodiments, the increase in pulmonary function occurs within one week of the administration of a MetAP2 inhibitor of the present disclosure, or pharmaceutically acceptable salt thereof. In some embodiments, the administration of a MetAP2 inhibitor of the present disclosure, or pharmaceutically acceptable salt thereof is after exposure to bleomycin.

In some embodiments, the administration of a MetAP2 inhibitor of the present disclosure, or pharmaceutically acceptable salt thereof, and nintedanib, or a pharmaceutically acceptable salt thereof, results in an increase in pulmonary function as indicated by a decrease in pulmonary congestion, decrease in pulmonary inflammation, an increase in SpO₂ values, a decrease in blood lactate levels, an increase in blood bicarbonate ion levels, a decrease in edema, a decrease in vascular leakiness and extracellular matrix deposition, a decrease in lung tissue resistance and stiffness, a decrease in parenchyma density, a decrease in pulmonary foci, a decrease in collagen content of the lung, and a decrease in respiratory rates as compared to a subject who was not administered a MetAP2 inhibitor of the present disclosure, pharmaceutically acceptable salt thereof, and nintedanib, or a pharmaceutically acceptable salt thereof. In some embodiments, the increase in pulmonary function occurs within one week of the administration of a MetAP2 inhibitor of the present disclosure, pharmaceutically acceptable salt thereof, and nintedanib, or a pharmaceutically acceptable salt thereof. In some embodiments, the administration of a MetAP2 inhibitor of the present disclosure, or pharmaceutically acceptable salt thereof, and nintedanib, or a pharmaceutically acceptable salt thereof, is after exposure to bleomycin.

In some embodiments, the administration of a MetAP2 inhibitor of the present disclosure, or pharmaceutically acceptable salt thereof, and pirfenidone, or a pharmaceutically acceptable salt thereof, results in an increase in pulmonary function as indicated by a decrease of pulmonary congestion, a decrease in pulmonary inflammation, an increase SpO₂ values, a decrease in blood lactate levels, an increase in blood bicarbonate ion levels, a decrease in edema, a decrease in vascular leakiness and extracellular matrix deposition, a decrease in lung tissue resistance and stiffness, a decrease in parenchyma density, a decrease in pulmonary foci, a decrease in collagen content of the lung, and a decrease in respiratory rates, as compared to a subject who was not administered a MetAP2 inhibitor of the present disclosure, or pharmaceutically acceptable salt thereof, and pirfenidone, or a pharmaceutically acceptable salt thereof. In some embodiments, the increase in pulmonary function occurs within one week of the administration of the MetAP2 inhibitor of the present disclosure, or pharmaceutically acceptable salt thereof, and pirfenidone, or a pharmaceutically acceptable salt thereof. In some embodiments, the administration of a MetAP2 inhibitor of the present disclosure, or pharmaceutically acceptable salt thereof, and pirfenidone, or a pharmaceutically acceptable salt thereof, is after exposure to bleomycin.

In some embodiments, the administration of a MetAP2 inhibitor of the present disclosure, or pharmaceutically acceptable salt thereof, inhibits the deposition of collagen in lung tissue.

In some embodiments, the administration of a MetAP2 inhibitor of the present disclosure, or pharmaceutically acceptable salt thereof, and nintedanib, or a pharmaceutically acceptable salt thereof, inhibits the deposition of collagen in lung tissue. In some embodiments, the administration of the combination of MetAP2, or a pharmaceutically acceptable salt thereof, and nintedanib, or a pharmaceutically acceptable salt thereof, results in an increased reduction of collagen deposition as compared to the administration MetAP2 alone.

In some embodiments, the administration of a MetAP2 inhibitor of the present disclosure, or pharmaceutically acceptable salt thereof, and pirfenidone, or a pharmaceutically acceptable salt thereof, inhibits the deposition of collagen in lung tissue.

In some embodiments, the administration of a MetAP2 inhibitor of the present disclosure, or pharmaceutically acceptable salt thereof, results in a reduction in the deposition of collagen in lung tissue, as compared to a subject who was not administered a MetAP2 inhibitor of the present disclosure, pharmaceutically acceptable salt thereof.

In some embodiments, the administration of a MetAP2 inhibitor of the present disclosure, or pharmaceutically acceptable salt thereof, and nintedanib, or a pharmaceutically acceptable salt thereof, results in a reduction in the deposition of collagen in lung tissue, as compared to a subject who was not administered a MetAP2 inhibitor of the present disclosure, pharmaceutically acceptable salt thereof, and nintedanib, or a pharmaceutically acceptable salt thereof.

In some embodiments, the administration of a MetAP2 inhibitor of the present disclosure, or pharmaceutically acceptable salt thereof, and pirfenidone, or a pharmaceutically acceptable salt thereof, results in a reduction in the deposition of collagen in lung tissue, as compared to a subject who was not administered a MetAP2 inhibitor of the present disclosure, or pharmaceutically acceptable salt thereof, and pirfenidone, or a pharmaceutically acceptable salt thereof.

In some embodiments, the administration of a MetAP2 inhibitor of the present disclosure, or pharmaceutically acceptable salt thereof, after exposure to bleomycin, results in a reduction in the deposition of collagen in lung tissue, as compared to a subject who was not administered a MetAP2 inhibitor of the present disclosure, or pharmaceutically acceptable salt thereof.

In some embodiments, the administration of a MetAP2 inhibitor of the present disclosure, or pharmaceutically acceptable salt thereof, and nintedanib, or a pharmaceutically acceptable salt thereof, after exposure to bleomycin, results in a reduction in the deposition of collagen in lung tissue, as compared to a subject who was not administered a MetAP2 inhibitor of the present disclosure, or pharmaceutically acceptable salt thereof, and nintedanib, or a pharmaceutically acceptable salt thereof.

In some embodiments, the administration of a MetAP2 inhibitor of the present disclosure, or pharmaceutically acceptable salt thereof, and nintedanib, or a pharmaceutically acceptable salt thereof, after exposure to bleomycin, results in a reduction in the deposition of collagen in lung tissue, as compared to a subject who was not administered a MetAP2 inhibitor of the present disclosure, or pharmaceutically acceptable salt thereof.

In some embodiments, the administration of a MetAP2 inhibitor of the present disclosure, or pharmaceutically acceptable salt thereof, and pirfenidone, or a pharmaceutically acceptable salt thereof, after exposure to bleomycin results in a reduction in the deposition of collagen in lung tissue, as compared to a subject who was not administered a MetAP2 inhibitor of the present disclosure, or pharmaceutically acceptable salt thereof, and pirfenidone, or a pharmaceutically acceptable salt thereof.

In some embodiments, the administration of a MetAP2 inhibitor of the present disclosure, or pharmaceutically acceptable salt thereof, and pirfenidone, or a pharmaceutically acceptable salt thereof, after exposure to bleomycin, results in a reduction in the deposition of collagen in lung tissue, as compared to a subject who was not administered a MetAP2 inhibitor of the present disclosure, or pharmaceutically acceptable salt thereof.

### MetAP2 Inhibitors

Any of the MetAP2 inhibitors described herein can be used in the kits, pharmaceutical compositions, uses and methods described herein.

In some aspects, a MetAP2 inhibitor can be Compound 1, or a pharmaceutically acceptable salt, analog, derivative, salt or ester thereof, wherein Compound 1 is represented by:

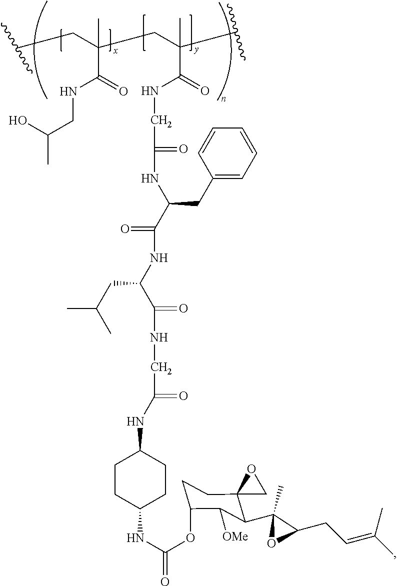

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, a MetAP2 inhibitor can be Compound 2, or a pharmaceutically acceptable salt, analog, derivative, salt or ester thereof, wherein Compound 2 is represented by:

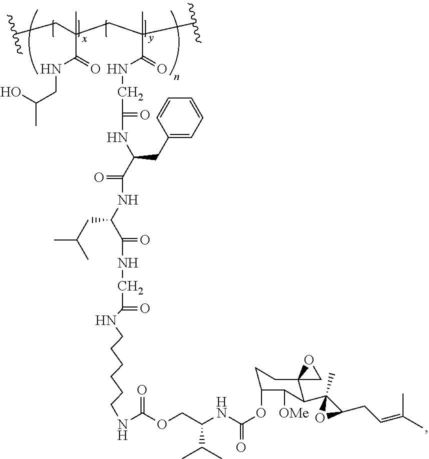

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, a MetAP2 inhibitor can be Compound 3, or a pharmaceutically acceptable salt, analog, derivative, salt or ester thereof, wherein Compound 3 is represented by:

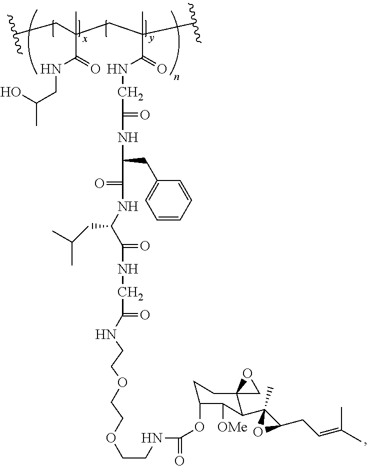

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, a MetAP2 inhibitor can be Compound 4, or a pharmaceutically acceptable salt, analog, derivative, salt or ester thereof, wherein Compound 4 is represented by:

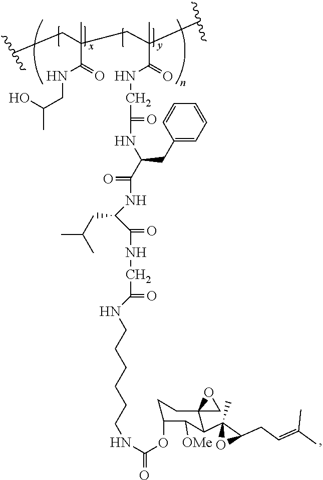

wherein x is in the range of 1 to about 450, y is in the range of 1 to about 30 and n is in the range of 1 to about 100. In some aspects, n is in the range of about 1 to about 90; about 1 to about 80; about 1 to about 70; about 1 to about 60; about 1 to about 55; or about 1 to about 50. In some aspects, the ratio of x to y can be in the range of about 30:1 to about 3:1.

In some aspects, the MetAP2 inhibitor can be:

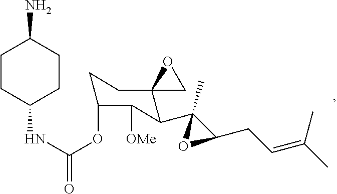

or a pharmaceutically acceptable salt, analog, derivative, salt or ester thereof.

In some aspects, the MetAP2 inhibitor can be:

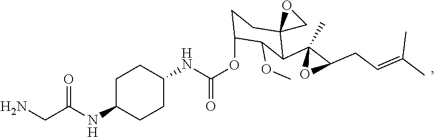

or a pharmaceutically acceptable salt, analog, derivative, salt or ester thereof.

In some aspects, the MetAP2 inhibitor can be:

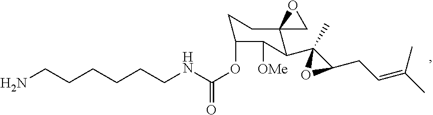

or a pharmaceutically acceptable salt, analog, derivative, salt or ester thereof.

In some aspects, the MetAP2 inhibitor can be:

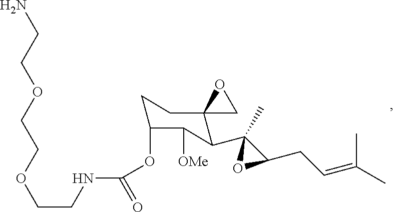

or a pharmaceutically acceptable salt, analog, derivative, salt or ester thereof.

In some aspects, the MetAP2 inhibitor can be:

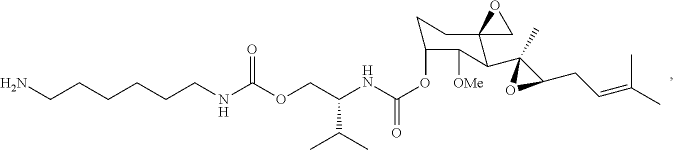

or a pharmaceutically acceptable salt, analog, derivative, salt or ester thereof.

In some aspects, the MetAP2 inhibitor can be selected from cis-(3aRS,9bRS)-7-(benzenesulfonylamino)-1,3a,4,9b-tetrahydro-2H-furo[2,3-c]chromene-6-carboxylic acid; cis-(3 aRS,9bRS)-7-[2-(3-diethylaminopropyl)-4-fluorobenzenesulfonyl-amino]-1,3a,4,9b-tetrahydro-2H-furo[2,3-c]chromene-6-carboxylic acid; cis-(3aRS,9bRS)-7-[2-(3-{pyrrolidin-1-yl}propyl)-4-fluorobenzenesulfonylamino]-1,3a,4,9b-tetrahydro-2H-furo[2,3-c]chromene-6-carboxylic acid; cis-(3aRS,9bRS)-7-[2-((Z)-3-diethylaminoprop-1-enyl)-4-fluorobenzenesulfonylamino]-1,3a,4,9b-tetrahydro-2H-furo[2,3-c]chromene-6-carboxylic acid; cis-(3aR,9bR)-7-[2-((Z)-3-diethylaminoprop-1-enyl)-4-fluoro-benzenesulfonylamino]-1,3a,4,9b-tetrahydro-2H-furo[2,3-c]chromene-6-carboxylic acid; cis-(3aS,9bS)-7-[2-((Z)-3-diethylaminoprop-1-enyl)-4-fluorobenzenesulfonylamino]-1,3a,4,9b-tetrahydro-2H-furo[2,3-c]chromene-6-carboxylic acid; 7-[2-((Z)-3-diethylaminoprop-1-enyl)-4-fluorobenzenesulfonylamino]-1,2-dihydrofuro[2,3-c]quinoline-6-carboxylic acid formate salt; 7-(benzenesulfonylamino))-1,2-dihydrofuro[2,3-c]quinoline-6-carboxylic acid formate salt; cis-(3aRS,9bRS)-7-[2-((Z)-3-diethylaminoprop-1-enyl)-4-fluorobenzenesulfonylamino]-1,2,3a,4,5,9b-hexahydrofuro[2,3-c]quinoline-6-carboxylic acid; (1 aRS, 7bSR)-5-[2-((Z)-3-diethylaminoprop-1-enyl)-4-fluorobenzenesulfonylamino]-1,1a,2,7b-tetrahydrocyclopropa[c]chromene-4-carboxylic acid; (1aR, 7bS)-5-[2-((Z)-3-diethylaminoprop-1-enyl)-4-fluorobenzenesulfonylamino]-1,1a,2,7b-tetrahydrocyclopropa[c]chromene-4-carboxylic acid; (1 aS, 7bR)-5-[2-((Z)-3-diethylaminoprop-1-enyl)-4-fluorobenzenesulfonylamino]-1,1a,2,7b-tetrahydrocyclopropa[c]chromene-4-carboxylic acid; (1aRS,7bSR)-5-[2-((Z)-3-diethylaminoprop-1-enyl)-4-fluorobenzenesulfonylamino]-7b-methyl-1, 1a,2,7b-tetrahydrocyclopropa[c]chromene-4-carboxylic acid; (laRS,7bSR)-5-[2-((E)-3-diethylaminoprop-1-enyl)-4-fluorobenzenesulfonylamino]-7b-methyl-1,1a,2,7b-tetrahydrocyclopropa[c]chromene-4-carboxylic acid; cis-(3aRS,9bRS)-7-[2-(4-dimethylamino-butylamino)-benzenesulfonylamino]-1,3a,4,9b-tetrahydro-2H-furo[2,3-c]chromene-6-carboxylic acid; (1 aR,7bS)-5-[2-(3-diethylaminopropyl)-4-fluorobenzenesulfonyl-amino]-1,1a,2,7b-tetrahydrocyclopropa[c]chromene-4-carboxylic acid; (laRS,7bSR)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1aR,7bS)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1 aS,7bR)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1aRS,7bSR)-5-[2 ((Z)-3-ethylaminoprop-1-enyl)-4-fluoro-benzenesulfonylamino]-1,1a,2,7b-tetrahydrocyclopropa-[c]chromene-4-carboxylic acid; (1aR, 7bS)-5-[2 ((Z)-3-ethylaminoprop-1-enyl)-4-fluorobenzenesulfonylamino]-1, 1a,2, 7b-tetrahydrocyclopropa[c]chromene-4-carboxylic acid; (laS,7bR)-5-[2 ((Z)-3-ethylaminoprop-1-enyl)-4-fluorobenzene-sulfonylamino]-1,1a,2,7b-tetrahydro-cyclopropa[c]chromene-4-carboxylic acid; (1aRS, 7bSR)-5-{2 [(Z)-3-(pyrrolidin-1-yl) prop-1-enyl]-4-fluorobenzenesulfonylamino}-1,1a,2,7b-tetrahydro-cyclopropa[c]chromene-4-carboxylic acid; (laR, 7bS)-5-{2 [(Z)-3-(pyrrolidin-1-yl) prop-1-enyl]-4-fluorobenzenesulfonyl-amino}-1,1a,2,7b-tetrahydro-cyclopropa[c]chromene-4-carboxylic acid; (1 aS, 7bR)-5-{2 [(Z)-3-(pyrrolidin-1-yl) prop-1-enyl]-4-fluorobenzenesulfonylamino}-1,1a,2,7b-tetrahydro-cyclopropa[c]chromene-4-carboxylic acid; (laRS,7bSR)-5-[2-(3-dimethylaminopropylamino)-benzenesulfonylamino]-1,1a,2,7b-tetrahydrocyclopropa[c]chromene-4-carboxylic acid; (1 aR, 7bS)-5-[2-(3-dimethylaminopropylamino)benzene-sulfonylamino]-1,1a,2,7b-tetrahydrocyclopropa[c]chromene-4-carboxylic acid; (1aS,7bR)-5-[2-(3-dimethylaminopropyl-amino)benzenesulfonylamino]-1,1a,2,7b-tetrahydrocyclopropa[c]chromene-4-carboxylic acid; (1aRS,7bSR)-5-[2-(4-dimethylaminobutylamino)benzenesulfonylamino]-1,1a,2,7b-tetrahydrocyclopropa[c]chromene-4-carboxylic acid; (1aR,7bS)-5-[2-(4-dimethylamino-butylamino)benzenesulfonylamino]-1,1a,2,7b-tetrahydrocyclopropa[c]chromene-4-carboxylic acid; (laS,7bR)-5-[2-(4-dimethylaminobutylamino]-1,1a,2,7b-tetrahydrocyclopropa[c]chromene-4-carboxylic acid; (1 aRS, 7bSR)-5-[2-(5-dimethylamino-pentylamino)benzene-sulfonylamino]-1,1a,2,7b-tetrahydrocyclopropa[c]chromene-4-carboxylic acid; (laRS, 7bSR)-5-{2 [(Z)-3-(propan-2-yl)aminoprop-1-enyl]-4-fluorobenzenesulfonyl-amino}-1, 1a,2,7b-tetrahydrocyclopropa[c]chromene-4-carboxylic acid; (1 aRS,7bSR)-5-{2 [(Z)-3-((S)-3-hydroxypyrrolidin-1-yl)aminoprop-1-enyl]-4-fluorobenzenesulfonylamino}-1,1a,2,7b-tetrahydrocyclopropa[c]chromene-4-carboxylic acid; (1 aRS,7bSR)-5-{2 [(Z)-3-((R)-3-hydroxypyrrolidin-1-yl)aminoprop-1-enyl]-4-fluorobenzene-sulfonylamino}-1, 1a,2, 7b-tetrahydro-cyclopropa[c]chromene-4-carboxylic acid; (1aRS,7bSR)-5-[2 ((Z)-4-diethylaminobut-1-enyl)-4-fluorobenzenesulfonyl-amino]-1, 1a,2, 7b-tetrahydrocyclopropa[c]chromene-4-carboxylic acid; (1aR, 7bS)-5-[2 ((Z)-4-diethylaminobut-1-enyl)-4-fluorobenzenesulfonyl-amino]-1,1a,2,7b-tetrahydrocyclopropa[c]chromene-4-carboxylic acid; (1 aS, 7bR)-5-[2 ((Z)-4-diethylaminobut-1-enyl)-4-fluorobenzenesulfonyl-amino]-1,1a,2,7b-tetrahydro-cyclopropa[c]chromene-4-carboxylic acid; (1 aRS,7bSR)-5-{2-[2-(4-ethylpiperazin-1-yl)-ethyl]-4-fluorobenzenesulfonylamino}-1,1a,2,7b-tetrahydrocyclopropa[c]chromene-4-carboxylic acid; (laRS,7bSR)-5-{2 [(Z)-3-(azetidin-1-yl) prop-1-enyl]-4-fluorobenzene-sulfonylamino}-1, 1a,2, 7b-tetrahydro-cyclopropa[c]chromene-4-carboxylic acid; (1aRS,7bSR)-5-{2 [(Z)-3-(3-hydroxy-azetidin-1-yl) prop-1-enyl]-4-fluorobenzene-sulfonylamino}-1,1a,2,7b-tetrahydrocyclopropa-[c]chromene-4-carboxylic acid; (1aRS,7bSR)-5-{2 [(Z)-3-(azetidin-1-yl)propyl]-4-fluorobenzenesulfonylamino}-1,1a,2,7b-tetrahydrocyclopropa[c]chromene-4-carboxylic acid; (1 aRS,7bSR)-5-[2 ((Z)-4-diethylaminobutyl)-4-fluorobenzenesulfonylamino]-1, 1a,2,7b-tetrahydrocyclopropa[c]chromene-4-carboxylic acid; (1 aRS,7bSR)-5-{2-[N-(4-dimethylaminobutyl)-N-methylamino]-benzenesulfonyl-amino}-1,1a,2, 7b-tetrahydrocyclopropa-[c]chromene-4-carboxylic acid; (1aRS,7bSR)-5-{2-[((S)-1-ethylpyrrolidin-3-ylcarbamoyl)-methyl]-4-fluoro-benzenesulfonyl-amino}-1,1a,2,7b-tetrahydrocyclopropa-[c]chromene-4-carboxylic acid; (1aRS,7bSR)-5-[2-(1-ethylazetidin-3-yl)-4-fluorobenzenesulfonylamino]-1,1a,2,7b-tetrahydro-cyclopropa[c]chromene-4-carboxylic acid; (1 aRS,7bSR)-5-{2-[((R)-1-ethylpyrrolidin-3-ylcarbamoyl)methyl]-4-fluorobenzenesulfonyl-amino}-1, 1a,2,7b-tetrahydro-cyclopropa[c]chromene-4-carboxylic acid; (1 aRS, 7bSR)-5-{2-[2-(pyrrolidin-1-yl)-ethyl]-4-fluorobenzenesulfonylamino}-1, 1a,2, 7b-tetrahydrocyclopropa[c]chromene-4-carboxylic acid; (1aRS,7bSR)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1aS,7bR)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1 aR, 7bS)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1aRS,7bSR)-5-{2-[((S)-1-ethylpyrrolidin-2-yl)carbonyl-aminomethyl]-4-fluorobenzene-sulfonylamino}-1, 1a,2, 7b-tetrahydrocyclopropa[c]chromene-4-carboxylic acid; (1 aRS, 7bSR)-5-[2-(4-dimethylaminobutyrylamino)-4-fluorobenzenesulfonyl-amino]-1, 1a,2, 7b-tetrahydrocyclopropa-[c]chromene-4-carboxylic acid; (1aRS,7bSR)-5-[2-((S)-1-ethyl-pyrrolidin-3-ylmethyl)-4-fluorobenzenesulfonyl-amino]-1,1a,2,7b-tetrahydrocyclopropa[c]chromene-4-carboxylic acid; (1aRS,7bSR)-5-[2-(3-dimethylaminopropylcarbamoyl)benzene-sulfonylamino]-1,1a,2,7b-tetrahydrocyclopropa[c]chromene-4-carboxylic acid; (1 aRS,7bSR)-5-(2-{[N-((S)-1-ethyl-pyrrolidin-3-yl)-N-methylcarbamoyl]methyl}-4-fluoro-benzenesulfonylamino)-1,1a,2,7b-tetrahydrocyclopropa-[c]chromene-4-carboxylic acid; (1 aRS, 7bSR)-5-(2-{[N-((R)-1-ethyl-pyrrolidin-3-yl)-N-methylcarbamoyl]methyl}-4-fluoro-benzenesulfonylamino)-1,1a,2,7b-tetrahydrocyclopropa-[c]chromene-4-carboxylic acid; (1 aRS, 7bSR)-5-{2-[2-((S)-1-ethylpyrrolidin-2-yl)ethylamino]-benzenesulfonyl-amino}-1,1a,2,7b-tetrahydrocyclopropa-[c]chromene-4-carboxylic acid; (1aRS,7bSR)-5-{2-[2-((R)-1-ethylpyrrolidin-2-yl)ethylamino]-benzenesulfonyl-amino}-1,1a,2,7b-tetrahydrocyclopropa-[c]chromene-4-carboxylic acid; (1aRS,7bSR)-5-[2-(3-N,N,-diethylaminopropylamino)benzene-sulfonylamino]-1, 1a,2,7b-tetrahydrocyclopropa[c]chromene-4-carboxylic acid; (1aRS,7bSR)-5-(2-{[((R)-1-ethylpyrrolidine-2-yl)carbonyl-amino]methyl}-4-fluorobenzenesulfonylamino)-1,1a,2, 7b-tetrahydrocyclopropa[c]chromene-4-carboxylic acid; (1aRS,7bSR)-5-{2-[(1-ethylazetidin-3-ylmethyl)amino]benzene-sulfonylamino}-1,1a,2,7b-tetrahydrocyclopropa[c]chromene-4-carboxylic acid; (1 aS, 7bR)-5-[2-((Z)-3-diethylaminoprop-1-enyl)benzenesulfonylamino]-1,1a,2,7b-tetrahydrocyclopropa[c]chromene-4-carboxylic acid; (1 aR, 7bS)-5-[2-((Z)-3-diethylaminoprop-1-enyl)benzenesulfonylamino]-1,1a,2,7b-tetrahydrocyclopropa[c]chromene-4-carboxylic acid; (1 aRS, 7bSR)-5-(2-{N-[((R)-1-ethylpyrrolidine-2-yl)carbonyl]-N-methyl-aminomethyl}-4-fluorobenzenesulfonylamino)-1, 1a,2, 7b-tetrahydrocyclopropa[c]chromene-4-carboxylic acid; (1 aRS,7bSR)-5-(2-{N-[((S)-1-ethylpyrrolidine-2-yl)carbonyl]-N-methylamino-methyl}-4-fluorobenzenesulfonylamino)-1, 1a,2, 7b-tetrahydrocyclopropa[c]chromene-4-carboxylic acid; (1 aRS,7bSR)-5-[2-(4-dimethylaminobutylamino)-4-fluorobenzenesulfonyl-amino]-1,1a,2,7b-tetrahydrocyclopropa[c]chromene-4-carboxylic acid; (1aRS,7bSR)-5-{2-[((R)-1-ethylpyrrolidin-3-ylmethyl)amino]-benzenesulfonylamino}-1,1a,2,7b-tetrahydrocyclopropa-[c]chromene-4-carboxylic acid; (1aRS, 7bSR)-5-{2-[((S)-1-ethylpyrrolidin-3-ylmethyl)amino]-benzenesulfonylamino}-1,1a,2,7b-tetrahydrocyclopropa-[c]chromene-4-carboxylic acid; (laRS,7bSR)-5-[2-(4-ethyl-2-oxopiperazin-1-ylmethyl)-4-fluorobenzene-sulfonylamino]-1, 1a,2,7b-tetrahydrocyclopropa[c]chromene-4-carboxylic acid; (1aRS,7bSR)-5-[2-(1-ethylpiperidin-4-ylmethyl)-4-fluoro-benzenesulfonylamino]-1,1a,2,7b-tetrahydrocyclopropa-[c]chromene-4-carboxylic acid; (1 aRS, 7bSR)-5-{2-[2-(1-ethylazetidin-3-yl)ethyl]-4-fluoro-benzenesulfonyl-amino}-1,1a,2,7b-tetrahydrocyclopropa[c]chromene-4-carboxylic acid; (1 aRS, 7bSR)-5-{2-[((S)-1-azabicyclo[2.2.2]oct-3-yl)amino]benzenesulfonyl-amino}-1,1a,2,7b-tetrahydrocyclopropa[c]chromene-4-carboxylic acid; (1 aRS,7bSR)-5-{2-[((R)-1-azabicyclo-[2.2.2]oct-3-yl)amino]benzenesulfonyl-amino}-1,1a,2, 7b-tetrahydrocyclopropa[c]chromene-4-carboxylic acid; (1 aRS,7bSR)-5-(2-{[((S)-1-ethylpyrrolidine-3-carbonyl)amino]methyl}-4-fluoro-benzenesulfonylamino)-1, 1a,2,7b-tetrahydrocyclopropa[c]chromene-4-carboxylic acid; (1 aRS,7bSR)-5-{2-[2-((R)-1-ethylpyrrolidin-3-ylamino)ethyl]-4-fluoro-benzenesulfonylamino}-1, 1a,2,7b-tetrahydrocyclopropa-[c]chromene-4-carboxylic acid; (1 aRS, 7bSR)-5-{2-[((R)-1-ethylpyrrolidin-3-yl)amino]-benzenesulfonylamino}-1,1a,2,7b-tetrahydrocyclopropa-[c]chromene-4-carboxylic acid; (1aRS,7bSR)-5-{2-[((S)-1-ethylpyrrolidin-3-yl)amino]-benzenesulfonylamino}-1,1a,2,7b-tetrahydrocyclopropa-[c]chromene-4-carboxylic acid; (1 aRS, 7bSR)-5-(2-{[((R)-1-ethylpyrrolidine-3-carbonyl)amino]-methyl)}-4-fluoro-benzenesulfonylamino)-1,1a,2,7b-tetrahydro-cyclopropa[c]chromene-4-carboxylic acid; (1aRS,7bSR)-5-[2-((Z)-3-diethylamino-2-methylprop-1-enyl)-4-fluorobenzene-sulfonylamino]-1,1a,2,7b-tetrahydrocyclopropa[c]chromene-4-carboxylic acid; (1 aRS, 7bSR)-5-{2-[2-((R)-1-ethylpyrrolidin-3-yl)ethylamino]-benzenesulfonylamino}-1,1a,2,7b-tetrahydrocyclopropa-[c]chromene-4-carboxylic acid; (1aRS, 7bSR)-5-{2-[2-((S)-1-ethylpyrrolidin-3-yl)ethylamino]-benzenesulfonyl-amino}-1,1a,2,7b-tetrahydrocyclopropa-[c]chromene-4-carboxylic acid; (1aR,7bS)-5-[2-((S)-1-ethylpyrrolidin-3-yloxymethyl)-4-fluoro-benzenesulfonylamino]-1,1a,2, 7b-tetrahydrocyclopropa-[c]chromene-4-carboxylic acid; (1 aR, 7bS)-5-[2-((R)-1-ethylpyrrolidin-3-yloxymethyl)-4-fluoro-benzenesulfonylamino]-1, 1a,2, 7b-tetrahydrocyclopropa-[c]chromene-4-carboxylic acid; (1aR, 7bS)-5-[2-(1-ethylpiperidin-3-ylmethyl)-4-fluorobenzene-sulfonylamino]-1,1a,2,7b-tetrahydrocyclopropa[c]chromene-4-carboxylic acid; (1aR, 7bS)-5-{2-[2-((R)-1-ethylpyrrolidin-2-yl)ethyl]-4-fluorobenzenesulfonyl-amino}-1,1a,2,7b-tetrahydrocyclopropa-[c]chromene-4-carboxylic acid; and pharmaceutically acceptable salts, stereoisomers, esters and prodrugs thereof.

In some aspects, a MetAP2 inhibitor can be selected from:

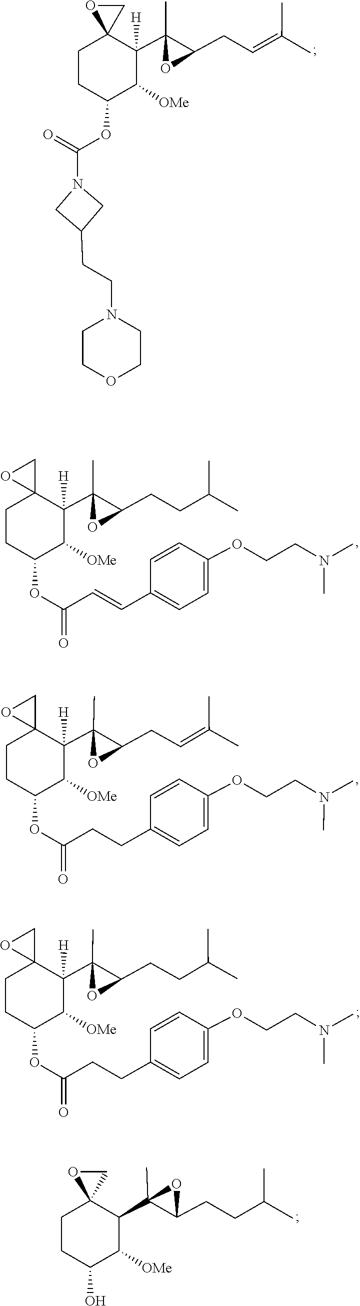 [SMILES: CO[C@@H]1[C@H](OC(=O)N2CC(CCN3CCOCC3)C2)CC[C@]2(CO2)[C@H]1[C@@]1(C)O[C@@H]1CC=C(C)C ; CO[C@@H]1[C@H](OC(=O)/C=C/c2ccc(OCCN(C)C)cc2)CCC2(CO2)[C@H]1[C@@]1(C)O[C@@H]1CCC(C)C ; CO[C@@H]1[C@H](OC(=O)CCc2ccc(OCCN(C)C)cc2)CCC2(CO2)[C@H]1[C@@]1(C)O[C@@H]1CC=C(C)C ; CO[C@@H]1[C@H](OC(=O)CCc2ccc(OCCN(C)C)cc2)CCC2(CO2)[C@H]1[C@@]1(C)O[C@@H]1CCC(C)C ; CO[C@@H]1[C@H](O)CC[C@]2(CO2)[C@H]1[C@@]1(C)O[C@@H]1CCC(C)C]

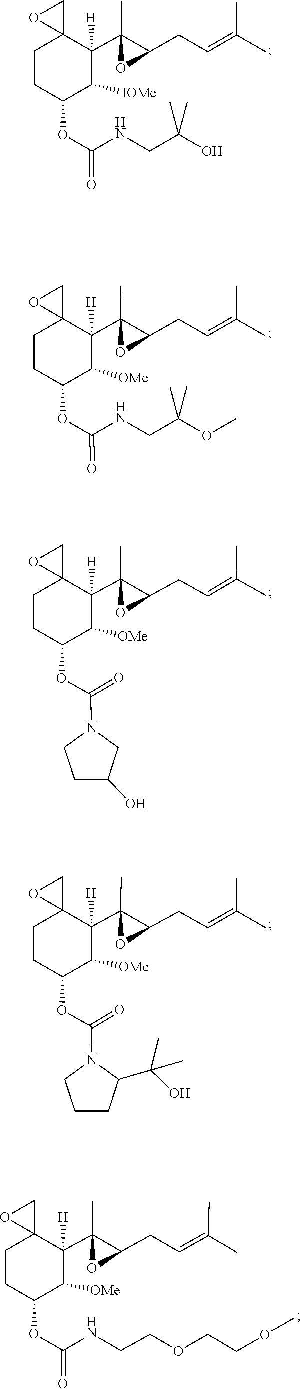

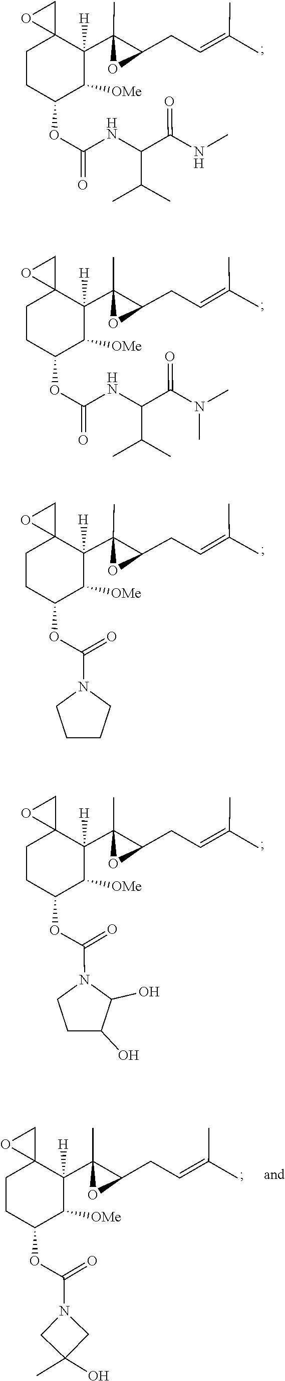 [SMILES: CNC(=O)C(NC(=O)O[C@@H]1CCC2(CO2)[C@@H]([C@@]2(C)O[C@@H]2CC=C(C)C)[C@@H]1OC)C(C)C ; CO[C@@H]1[C@H](OC(=O)NC(C(=O)N(C)C)C(C)C)CCC2(CO2)[C@H]1[C@@]1(C)O[C@@H]1CC=C(C)C ; CO[C@@H]1[C@H](OC(=O)N2CCCC2)CCC2(CO2)[C@H]1[C@@]1(C)O[C@@H]1CC=C(C)C ; CO[C@@H]1[C@H](OC(=O)N2CCC(O)C2O)CCC2(CO2)[C@H]1[C@@]1(C)O[C@@H]1CC=C(C)C ; CO[C@@H]1[C@H](OC(=O)N2CC(C)(O)C2)CCC2(CO2)[C@H]1[C@@]1(C)O[C@@H]1CC=C(C)C]

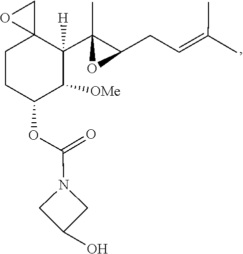 [SMILES: CO[C@@H]1[C@H](OC(=O)N2CC(O)C2)CCC2(CO2)[C@H]1[C@@]1(C)O[C@@H]1CC=C(C)C]

or a pharmaceutically acceptable salt, analog, derivative, salt or ester thereof.

In some aspects, a MetAP2 inhibitor can be:

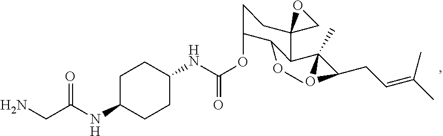

or a pharmaceutically acceptable salt, analog, derivative, salt or ester thereof.

In some aspects, a MetAP2 inhibitor can be administered by subcutaneous injection (SC). In some aspects, a MetAP2 inhibitor can be administered by subcutaneous injection to the mid-abdominal (per-umbilical area). In some aspects, a subcutaneous injection of a MetAP2 inhibitor can be administered over an about 30 to about 45 second timeframe at a constant injection rate. In some aspects, the maximum injection volume of a MetAP2 inhibitor is less than about 1.7 ml.

In some aspects, a MetAP2 inhibitor can be administered about every four days (Q4D).

In some aspects, a MetAP2 inhibitor can be administered about once every day (QD), about once every two days (Q2D), about once every three days (Q3D), about once every four days (Q4D), about once every 5 days (Q5D), about once every 6 days (Q6D), about once every 7 days (Q7D), about once every 8 days (Q8D), about once every 9 days (Q9D), about once every 10 days (Q10D), about once every 11 days (Q11D), about once every 12 days (Q12D), about once every 13 days (Q13D), about once every 14 days (Q14D), or about once every 15 days (Q15D). In some aspects, a MetAP2 inhibitor can be administered about once every 7 days (Q7D). In some aspects, a MetAP2 inhibitor can be administered about once every 14 days (Q14D).

In some aspects, a MetAP2 inhibitor can be administered in an amount of about 1 mg/m², or about 2 mg/m², or about 3 mg/m², or about 4 mg/m², or about 5 mg/m², or about 6 mg/m², or about 7 mg/m², or about 8 mg/m², or about 9 mg/m², or about 10 mg/m², or about 11 mg/m², or about 12 mg/m², or about 13 mg/m², or about 14 mg/m², or about 15 mg/m², or about 16 mg/m², or about 17 mg/m², or about 18 mg/m², or about 19 mg/m², or about 20 mg/m², or about 21 mg/m², or about 22 mg/m², or about 23 mg/m², or about 24 mg/m², or about 25 mg/m², or about 26 mg/m², or about 27 mg/m², or about 28 mg/m², or about 29 mg/m², or about 30 mg/m², or about 31 mg/m², or about 32 mg/m², or about 33 mg/m², or about 34 mg/m², or about 35 mg/m², or about 36 mg/m², or about 37 mg/m², or about 38 mg/m², or about 39 mg/m², or about 40 mg/m², or about 41 mg/m², or about 42 mg/m², or about 43 mg/m², or about 44 mg/m², or about 45 mg/m², or about 46 mg/m², or about 47 mg/m², or about 48 mg/m², or about 49 mg/m², or about 50 mg/m², or about 51 mg/m², or about 52 mg/m², or about 53 mg/m², or about 54 mg/m², or about 55 mg/m², or about 56 mg/m², or about 57 mg/m², or about 58 mg/m², or about 59 mg/m², or about 60 mg/m², or about mg/m², or about 61 mg/m², or about 62 mg/m², or about 63 mg/m², or about 64 mg/m², or about 65 mg/m², or about 66 mg/m², or about 67 mg/m², or about 68 mg/m², or about 69 mg/m², or about 70 mg/m², or about 81 mg/m², or about 82 mg/m², or about 83 mg/m², or about 84 mg/m², or about 85 mg/m², or about 86 mg/m², or about 87 mg/m², or about 88 mg/m², or about 89 mg/m², or about 90 mg/m², or about 91 mg/m², or about 92 mg/m², or about 93 mg/m², or about 94 mg/m², or about 95 mg/m², or about 96 mg/m², or about 97 mg/m², or about 98 mg/m², or about 99 mg/m², or about 100 mg/m².

In some aspects, a MetAP2 inhibitor can be administered in an amount of about 49 mg/m². In some aspects, a MetAP2 inhibitor can be administered in an amount of about 36 mg/m². In some aspects, a MetAP2 inhibitor can be administered in an amount of about 65 mg/m². In some aspects, a MetAP2 inhibitor can be administered in an amount of about 27 mg/m².

In some aspects, a MetAP2 inhibitor can be administered in an amount of about 49 mg/m². In some aspects, a MetAP2 inhibitor can be administered in an amount of about 39 mg/m² to about 59 mg/m². In some aspects, a MetAP2 inhibitor can be administered in an amount of about 44 mg/m² to about 54 mg/m².

In some aspects, a MetAP2 inhibitor can be administered in an amount of about 36 mg/m². In some aspects, a MetAP2 inhibitor can be administered in an amount of about 26 mg/m² to about 49 mg/m². In some aspects, a MetAP2 inhibitor can be administered in an amount of about 31 mg/m² to about 65 mg/m².

In some aspects, a MetAP2 inhibitor can be administered in an amount of about 65 mg/m². In some aspects, a MetAP2 inhibitor can be administered in an amount of about 55 mg/m² to about 75 mg/m². In some aspects, a MetAP2 inhibitor can be administered in an amount of about 60 mg/m² to about 70 mg/m².

In some aspects, a therapeutically effective amount of a MetAP2 inhibitor can be about 1 mg/m², or about 2 mg/m², or about 3 mg/m², or about 4 mg/m², or about 5 mg/m², or about 6 mg/m², or about 7 mg/m², or about 8 mg/m², or about 9 mg/m², or about 10 mg/m², or about 11 mg/m², or about 12 mg/m², or about 13 mg/m², or about 14 mg/m², or about 15 mg/m², or about 16 mg/m², or about 17 mg/m², or about 18 mg/m², or about 19 mg/m², or about 20 mg/m², or about 21 mg/m², or about 22 mg/m², or about 23 mg/m², or about 24 mg/m², or about 25 mg/m², or about 26 mg/m², or about 27 mg/m², or about 28 mg/m², or about 29 mg/m², or about 30 mg/m², or about 31 mg/m², or about 32 mg/m², or about 33 mg/m², or about 34 mg/m², or about 35 mg/m², or about 36 mg/m², or about 37 mg/m², or about 38 mg/m², or about 39 mg/m², or about 40 mg/m², or about 41 mg/m², or about 42 mg/m², or about 43 mg/m², or about 44 mg/m², or about 45 mg/m², or about 46 mg/m², or about 47 mg/m², or about 48 mg/m², or about 49 mg/m², or about 50 mg/m², or about 51 mg/m², or about 52 mg/m², or about 53 mg/m², or about 54 mg/m², or about 55 mg/m², or about 56 mg/m², or about 57 mg/m², or about 58 mg/m², or about 59 mg/m², or about 60 mg/m², or about mg/m², or about 61 mg/m², or about 62 mg/m², or about 63 mg/m², or about 64 mg/m², or about 65 mg/m², or about 66 mg/m², or about 67 mg/m², or about 68 mg/m², or about 69 mg/m², or about 70 mg/m², or about 81 mg/m², or about 82 mg/m², or about 83 mg/m², or about 84 mg/m², or about 85 mg/m², or about 86 mg/m², or about 87 mg/m², or about 88 mg/m², or about 89 mg/m², or about 90 mg/m², or about 91 mg/m², or about 92 mg/m², or about 93 mg/m², or about 94 mg/m², or about 95 mg/m², or about 96 mg/m², or about 97 mg/m², or about 98 mg/m², or about 99 mg/m², or about 100 mg/m².

In some aspects, a therapeutically effective amount of a MetAP2 inhibitor can be about 49 mg/m². In some aspects, a therapeutically effective amount of a MetAP2 inhibitor can be about 39 mg/m² to about 59 mg/m². In some aspects, a therapeutically effective amount of a MetAP2 inhibitor can be about 44 mg/m² to about 54 mg/m².

In some aspects, a therapeutically effective amount of a MetAP2 inhibitor can be about 36 mg/m². In some aspects, a therapeutically effective amount of a MetAP2 inhibitor can be about 26 mg/m² to about 49 mg/m². In some aspects, a therapeutically effective amount of a MetAP2 inhibitor can be about 31 mg/m² to about 49 mg/m².

In some aspects, a therapeutically effective amount of a MetAP2 inhibitor can be about 65 mg/m². In some aspects, a therapeutically effective amount of a MetAP2 inhibitor can be about 55 mg/m² to about 75 mg/m². In some aspects, a therapeutically effective amount of a MetAP2 inhibitor can be about 60 mg/m² to about 70 mg/m².

In some aspects, a MetAP2 inhibitor can be administered in an amount of about 10 mg, or about 20 mg, or about 30 mg, or about 40 mg, or about 50 mg, or about 60 mg, or about 70 mg, or about 80 mg, or about 90 mg, or about 100 mg or about 110 mg, or about 120 mg, or about 130 mg, or about 140 mg, or about 150 mg, or about 160 mg, or about 170 mg, or about 180 mg, or about 190 mg, or about 200 mg. In some aspects a MetAP2 inhibitor can be administered in an amount of about 80 mg. In some aspects, a MetAP2 inhibitor can be administered in an amount of about of about 70 mg to about 90 mg. In some aspects, a MetAP2 inhibitor can be administered in an amount of about 75 mg to about 85 mg.

In some aspects, a therapeutically effective amount of a MetAP2 inhibitor can be about 10 mg, or about 20 mg, or about 30 mg, or about 40 mg, or about 50 mg, or about 60 mg, or about 70 mg, or about 80 mg, or about 90 mg, or about 100 mg. In some aspects a therapeutically effective amount of a MetAP2 inhibitor can be about 80 mg. In some aspects, a therapeutically effective amount of a MetAP2 inhibitor can be about of about 70 mg to about 90 mg. In some aspects, a therapeutically effective amount of a MetAP2 inhibitor can be about 75 mg to about 85 mg.

### Pirfenidone

As would be appreciated by the skilled artisan, pirfenidone has the following chemical structure:

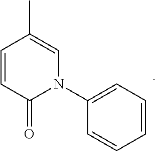

As would be appreciated by the skilled artisan, it is understood that pirfenidone may be identified by any of the following names: 2 (1H)-Pyridinone, 5-methyl-1-phenyl-; 5-Methyl-1-phenyl-2 (1H)-pyridinone; 5-Methyl-1-phenyl-2 (1H)-pyridone; AMR 69; 5-Methyl-1-phenyl-1H-pyridin-2-one; Deskar; Pirespa; Esbriet; RG6062; RG 6062; RG-6062. As would be appreciated by the skilled artisan, Pirfenidone may be identified as CAS No. 53179-13-8.

In some aspects, a pirfenidone can be administered orally.

In some aspects, pirfenidone can be administered in an amount of about 75 mg, or about 100 mg, or about 125 mg, or about 150 mg, or about 175 mg, or about 200 mg, or about 225 mg, or about 250 mg, or about 267 mg, or about 275 mg, or about 300 mg, or about 325 mg, or about 350 mg, or about 375 mg, or about 400 mg, or about 425 mg, or about 450 mg, or about 500 mg, or about 525 mg, or about 550 mg, or about 575 mg, or about 600 mg, or about 625 mg, or about 650 mg, or about 675 mg, or about 700 mg, or about 725 mg, or about 750 mg, or about 775 mg, or about 800 mg, or about 801 mg, or about 825 mg, or about 850 mg, or about 875 mg, or about 900 mg, or about 925 mg, or about 950 mg, or about 975 mg, or about 1000 mg, or about 1025 mg, or about 1050 mg, or about 1075 mg, or about 1100 mg, or about 1125 mg, or about 1150 mg, or about 1175 mg, or about 1200 mg, or about 1225 mg, or about 1250 mg, or about 1267 mg, or about 1275 mg, or about 1300 mg, or about 1325 mg, or about 1350 mg, or about 1375 mg, or about 1400 mg, or about 1425 mg, or about 1450 mg, or about 1500 mg, or about 1525 mg, or about 1550 mg, or about 1575 mg, or about 1600 mg, or about 1625 mg, or about 1650 mg, or about 1675 mg, or about 1700 mg, or about 1725 mg, or about 1750 mg, or about 1775 mg, or about 1800 mg, or about 1801 mg, or about 1825 mg, or about 1850 mg, or about 1875 mg, or about 1900 mg, or about 1925 mg, or about 1950 mg, or about 1975 mg, or about 2000 mg, or about 2025 mg, or about 2050 mg, or about 2075 mg, or about 2100 mg, or about 2125 mg, or about 2150 mg, or about 2175 mg, or about 2200 mg, or about 2225 mg, or about 2250 mg, or about 2275 mg, or about 2300 mg, or about 2325 mg, or about 2350 mg, or about 2375 mg, or about 2400 mg, or about 2403 mg, or about 2425 mg, or about 2450 mg, or about 2475 mg.

In some aspects, pirfenidone can be administered in an amount of about 801 mg. In aspects wherein pirfenidone is administered in an amount of about 801 mg, the amount can be administered by orally administering three capsules of 267 mg.

In some aspects, pirfenidone can be administered in an amount of about 2403 mg per day. In aspects wherein pirfenidone is administered in an amount of about 2403 mg per day, the amount can be administered by orally administering three capsules of 267 mg three times a day.

In some aspects, a therapeutically effective amount of pirfenidone can be any of the pirfenidone amounts described herein.

In some aspects, pirfenidone can be administered as a pharmaceutical composition, wherein the pharmaceutical composition comprises at least one of microcrystalline cellulose, croscarmellose sodium, povidone, magnesium stearate, gelatin and titanium dioxide.

In some aspects, pirfenidone can be administered about once a day. In some aspects, pirfenidone can be administered about twice a day. In some aspects, pirfenidone can be administered about three times a day.

### Nintedanib

As would be appreciated by the skilled artisan, Nintedanib has the following chemical structure:

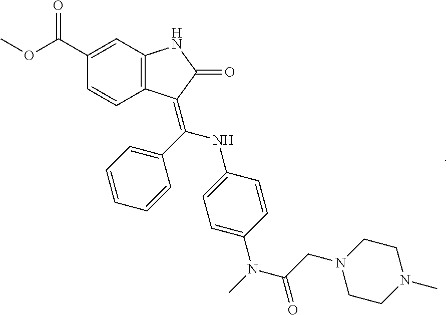

As would be appreciated by the skilled artisan, it is understood that nintedanib may be identified by any of the following names: 1H-Indole-6-carboxylic acid, 2,3-dihydro-3[[[4-[methyl[2-(4-methyl-1-piperazinyl)acetyl]amino]phenyl]amino]phenylmethylene]-2-oxo-, methyl ester, (3Z)-; 1H-Indole-6-carboxylic acid, 2,3-dihydro-3-[[4-[methyl[(4-methyl-1-piperazinyl)acetyl]amino]phenyl]amino]phenylmethylene]-2-oxo-, methyl ester, (3Z)-; BIBF 1120, Vargatef, Ofev, Methyl (Z)-3-[[[4-[N-methyl-2-(4-methylpiperazin-1-yl)acetamido]phenyl]amino](phenyl)methylene]-2-oxoindoline-6-carboxylate; Methyl (Z)-3-[[[4-(N-methyl-2-(4-methylpiperazin-]-yl)acetamido)phenyl)amino) (phenyl)methylene)-2-oxoindoline-6-carboxylate. As would be appreciated by the skilled artisan, Pirfenidone may be identified as CAS No. 656247-17-5.

In some aspects, nintedanib can be administered in the form of an ethanesulfonate salt, i.e. Nintedanib esylate (CAS No. 656247-18-6).

In some aspects, a nintedanib can be administered orally.

In some aspects, nintedanib can be administered in an amount of about 75 mg, or about 100 mg, or about 125 mg, or about 150 mg, or about 175 mg, or about 200 mg, or about 225 mg, or about 250 mg, or about 267 mg, or about 275 mg, or about 300 mg, or about 325 mg, or about 350 mg, or about 375 mg, or about 400 mg, or about 425 mg, or about 450 mg, or about 500 mg, or about 525 mg, or about 550 mg, or about 575 mg, or about 600 mg, or about 625 mg, or about 650 mg, or about 675 mg, or about 700 mg, or about 725 mg, or about 750 mg, or about 775 mg, or about 800 mg, or about 801 mg, or about 825 mg, or about 850 mg, or about 875 mg, or about 900 mg, or about 925 mg, or about 950 mg, or about 975 mg, or about 1000 mg, or about 1025 mg, or about 1050 mg, or about 1075 mg, or about 1100 mg, or about 1125 mg, or about 1150 mg, or about 1175 mg, or about 1200 mg, or about 1225 mg, or about 1250 mg, or about 1267 mg, or about 1275 mg, or about 1300 mg, or about 1325 mg, or about 1350 mg, or about 1375 mg, or about 1400 mg, or about 1425 mg, or about 1450 mg, or about 1500 mg, or about 1525 mg, or about 1550 mg, or about 1575 mg, or about 1600 mg, or about 1625 mg, or about 1650 mg, or about 1675 mg, or about 1700 mg, or about 1725 mg, or about 1750 mg, or about 1775 mg, or about 1800 mg, or about 1801 mg, or about 1825 mg, or about 1850 mg, or about 1875 mg, or about 1900 mg, or about 1925 mg, or about 1950 mg, or about 1975 mg, or about 2000 mg, or about 2025 mg, or about 2050 mg, or about 2075 mg, or about 2100 mg, or about 2125 mg, or about 2150 mg, or about 2175 mg, or about 2200 mg, or about 2225 mg, or about 2250 mg, or about 2275 mg, or about 2300 mg, or about 2325 mg, or about 2350 mg, or about 2375 mg, or about 2400 mg, or about 2403 mg, or about 2425 mg, or about 2450 mg, or about 2475 mg.

In some aspects, nintedanib can be administered in an amount of about 150 mg.

In some aspects, nintedanib can be administered in an amount of about 100 mg.

In some aspects, nintedanib can be administered in an amount of about 300 mg per day. In aspects wherein nintedanib is administered in an amount of about 300 mg per day, the amount can be administered by orally administering one capsule of 150 mg, twice a day. In some aspects, the two capsules can be administered about 12 hours apart.

In some aspects, nintedanib can be administered in an amount of about 200 mg per day. In aspects wherein nintedanib is administered in an amount of about 200 mg per day, the amount can be administered by orally administering one capsule of 100 mg, twice a day. In some aspects, the two capsules can be administered about 12 hours apart.

In some aspects, a therapeutically effective amount of nintedanib can be any of the nintedanib amounts described herein.

In some aspects, nintedanib can be administered as a pharmaceutical composition, wherein the pharmaceutical composition comprises at least one of triglycerides, hard fat, lecithin, gelatin, glycerol, titanium dioxide, red ferric oxide, yellow ferric oxide, black ink.

In some aspects, nintedanib can be administered about once a day. In some aspects, nintedanib can be administered about twice a day. In some aspects, nintedanib can be administered about three times a day.

### Diseases and Disorders

In some aspects, an interstitial lung disease can be pulmonary fibrosis.

In some embodiments, the pulmonary fibrosis results from an interstitial lung disease.

As would be appreciated by the skilled artisan, pulmonary fibrosis is a disease that occurs when lung tissue becomes damaged and scarred. The thickened, stiff tissue that results from the damage and scarring makes it more difficult for a subject's lungs to work properly.

In some aspects, the interstitial lung disease can be one or more of Asbestosis, COVID-19-related pulmonary fibrosis, Drug-induced pulmonary fibrosis, pneumonitis, Hypersensitivity Pneumonitis (HP), Idiopathic pulmonary fibrosis (IPF), Idiopathic Non-specific interstitial pneumonia (NSIP), Pneumoconiosis, Rheumatoid Arthritis Interstitial Lung Disease (RA-ILD), Sarcoidosis, Silicosis, pulmonary edema, pleural effusion, and systemic sclerosis. In some aspects, the pulmonary fibrosis is IPF.

In some aspects, an interstitial lung disease can be a treatment-induced interstitial lung disease. A treatment-induced interstitial lung disease is an interstitial lung disease that is caused by the administration or one or more treatments to a subject (i.e. the interstitial lung disease is an adverse side-effect of the one or more treatments; see Schwaiblmair et al; Camus et al.; Conte et al.; Skeoch et al. Drug-Induced Interstitial Lung Disease: A Systematic Review, 2018, J. Clin. Med. 7 (10): 356; and Spagnolo et al. Drug-induced interstitial lung disease, 2022, European Respiratory Journal, 60 (3), each of the aforementioned publications are incorporated by reference in their entireties for all purposes). In some aspects, a treatment-induced interstitial lung disease can be treatment-induced pulmonary fibrosis. In some aspects, a treatment-induced interstitial lung disease can be treatment-induced pneumonitis.

In some embodiments, the interstitial lung disease is drug induced.

In some aspects, the one or more treatments that induce an interstitial lung disease can be any treatment known in the art to include an interstitial lung disease (see Schwaiblmair et al; Skeoch et al.; Spagnolo et al.; Camus et al.; and Conte et al.).

In some aspects, the one or more treatments that induce interstitial lung disease can comprise at least one of Acetylsalicylic acid, Amphotericin B, Amiodarone, Azathioprine, Beta blockers, Carbamazepine, Clarithromycin, Diclofenac, Granulocyte colony stimulating factor, Phenytoin, Fluoxetine, Hydralazine, Levofloxacin, Contrast media, Minocycline, Naproxen, Nitrofurantoin, Gold, Paracetamol, Penicillamine, Penicillins, Statins, Sulfasalazine, Abemaciclib, Palbociclib, Ribociclib, Alectinib, Crizotinib, Ceritinib Nivolumab, Pembrolizumab, Atezolizumab, Durvalumab, Ipilimumab, Osimertinib, Vinorelbine, Paclitaxel, Docetaxel, Bleomycin, Gemcitabine, Erlotinib, Gefitinib, Panitumumab, Cetuximab, Everolimus, Temsirolimus, Sirolimus, Ipilimumab, Nivolumab, Irinotecan, Rituximab, Imatinib, Pemetrexed, Granulocyte colony stimulating factor, Methotrexate, Tumour necrosis factor inhibitors, Leflunomide, Amiodarone, Bepridil, Statins, Nitrofurantoin, Daptomycin, and Interferon.

In some aspects, the one or more treatments that induce interstitial lung disease can comprise at least one of an anti-epidermal growth factor receptor agent, anti-BRAF agent, a cyclin-dependent kinase 4/6 inhibitor, a poly (ADP-ribose) polymerase inhibitor, an immune check-point inhibitor, a PD-1 inhibitor, an EGFR inhibitor, a HER2 inhibitor, a BRC/ABL tyrosine kinase inhibitor, an ALK inhibitor, a BRAF inhibitor, a PI3K inhibitor, a FLT3 inhibitor, a TRK/ROS1 inhibitors, a VEGFR inhibitor, a CDK4/6 inhibitor, an mTOR inhibitor, and a PARP inhibitor.

In some aspects, the one or more treatments that induce interstitial lung disease can be comprise the administration of at least one anti-cancer agent.

In some aspects, an anti-cancer agent can comprise an antibody-drug conjugate.

In some aspects, an anti-cancer agent can comprise irinotecan. In some aspects, an anti-cancer agent can comprise an analogue or irinotecan. In some aspects, an anti-cancer agent can comprise a derivative of irinotecan. In some aspects, an anti-cancer agent can comprise an antibody-drug conjugate, wherein the antibody drug conjugate comprises irinotecan, an analogue of irinotecan, or a derivative of irinotecan.

In some aspects, an anti-cancer agent can comprise an antibody-drug conjugate, wherein the antibody-drug conjugate comprises deruxtecan.

In some aspects, an anti-cancer agent can comprise Enhertu (Trastuzumab deruxtecan).

In some aspects, an anti-cancer agent can comprise at least one of Abemaciclib, Palbociclib, Ribociclib, Alectinib, Crizotinib, Ceritinib Nivolumab, Pembrolizumab, Atezolizumab, Durvalumab, Ipilimumab, Osimertinib, Vinorelbine, Paclitaxel, Docetaxel, Bleomycin, Gemcitabine, Erlotinib, Gefitinib, Panitumumab, Cetuximab, Everolimus, Temsirolimus, Sirolimus, Ipilimumab, Nivolumab, Irinotecan, Rituximab, Imatinib, Pemetrexed and Granulocyte colony stimulating factor.

In some aspects, an anti-cancer agent can comprise at least one of an anti-epidermal growth factor receptor agent, anti-BRAF agent, a cyclin-dependent kinase 4/6 inhibitor, a poly (ADP-ribose) polymerase inhibitor, an immune check-point inhibitor, a PD-1 inhibitor, an EGFR inhibitor, a HER2 inhibitor, a BRC/ABL tyrosine kinase inhibitor, an ALK inhibitor, a BRAF inhibitor, a PI3K inhibitor, a FLT3 inhibitor, a TRK/ROS1 inhibitors, a VEGFR inhibitor, a CDK4/6 inhibitor, an mTOR inhibitor, and a PARP inhibitor.

In some aspects, the one or more treatments that induce interstitial lung disease can be comprise the administration of at least one anti-cancer agent.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, leukemia and germ cell tumors. More particular examples of such cancers include adrenocortical carcinoma, bladder urothelial carcinoma, breast cancers including Her2 positive or Her2 low, triple-negative, hormone receptor positive, invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary′ cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, cancers that express the human epidermal growth factor family receptors (e.g., Her2), or tumors that express the Trop2 receptor, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

In some aspects, the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

In some aspects, the cancer is breast cancer.

In some aspects, the breast cancer is metastatic breast cancer. As used herein, metastatic breast cancer is stage III or IV breast cancer that has spread to another part of the body, including, but not limited to, the liver, brain, bones, etc.

In some aspects, the breast cancer is human epidermal growth factor 2 (HER2)-negative breast cancer.

In some aspects, the breast cancer is HR+HER2-breast cancer.

In some aspects, the breast cancer can be a Luminal A breast cancer. In some aspects, the breast cancer can be a Luminal B breast cancer. In some aspects, the breast cancer can be a triple negative or basal-like breast cancer. In some aspects the breast cancer can be a HER2-enriched breast cancer.

In some aspects, the cancer is a head and neck cancer.

In some aspects, the cancer is a non-small cell lung cancer.

In some aspects, the cancer is a brain cancer. In some aspects, the brain cancer can be a recurring brain metastasis.

In some aspects, the cancer is a squamous cell carcinoma.

In some aspects, the cancer is a central nervous system tumor.

In some aspects, the cancer is liposarcoma.

In some aspects, the cancer is endometrial carcinoma.

In some aspects, the cancer is a neuroendocrine tumor.

In some aspects, the cancer is a small cell lung cancer (SCLC).

### Subjects

In some aspects, the subject in need thereof is an animal. In some aspects, the animal can be a mammal. In some aspects, the subject in need thereof is a human.

In some aspects, the subject in need thereof is a human of 18 years or older, or of 25 years or older, or of 50 years or older, or of 60 years or older, or of 65 years or older, or of 70 years or older, or of 75 years or older, or of 80 years or older, or of 85 years or older.

### General Definitions

Certain compounds of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure. Any representation of a particular isomer is merely exemplary (e.g., the exemplification of a trans-isomer, also encompasses a cis-isomer).

If, for instance, a particular enantiomer of a compound of the present disclosure is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this disclosure include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

As used herein, the term “temporal proximity” refers to that administration of one therapeutic agent (e.g., a MetAP2 inhibitor compound disclosed herein) occurs within a time period before or after the administration of another therapeutic agent (e.g., pirfenidone or nintedanib), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and/or complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of autoimmune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; decreased mortality at a given point of time following treatment; and/or improvement in one of the following symptoms: congestion, fluid/edema, wheezing, coughing, hypoxemia, low oxygen saturation, lung stiffness, shortness of breath, shortness of breath during exercise, dry hacking cough, fast shallow breathing, weight loss, tiredness, aching joints, aching muscles and clubbing.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “combination therapy” or “co-therapy” includes the administration of a compound of the present disclosure, or a pharmaceutically acceptable salt, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

It is to be understood that the present disclosure also provides pharmaceutical compositions comprising any compound described herein in combination with at least one pharmaceutically acceptable excipient or carrier.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

### Examples

The methods and combinations described herein are based on the surprising discovery that Compound 1 of the present disclosure significantly improved lung histology and pulmonary function in aged, male, bleomycin-treated mice. The combination of Compound 1, with nintedanib had a significantly greater effect on functional (PenH, SpO2, lung stiffness) and histologic (tissue density, fibrotic foci) endpoints relative to either compound alone. A study in young, female mice with bleomycin-induced disease showed benefit of Compound 1 plus nintedanib relative to either agent alone (lung weight, tissue density, fibrotic foci). Compound 1 also increased plasma levels of adiponectin. These findings, as well as others, a described in more detail in the examples presented infra.

### Example 1—Treatment of Pulmonary Fibrosis Using Compound 1 and Combinations of Compound 1 and Nintedanib or Pirfenidone

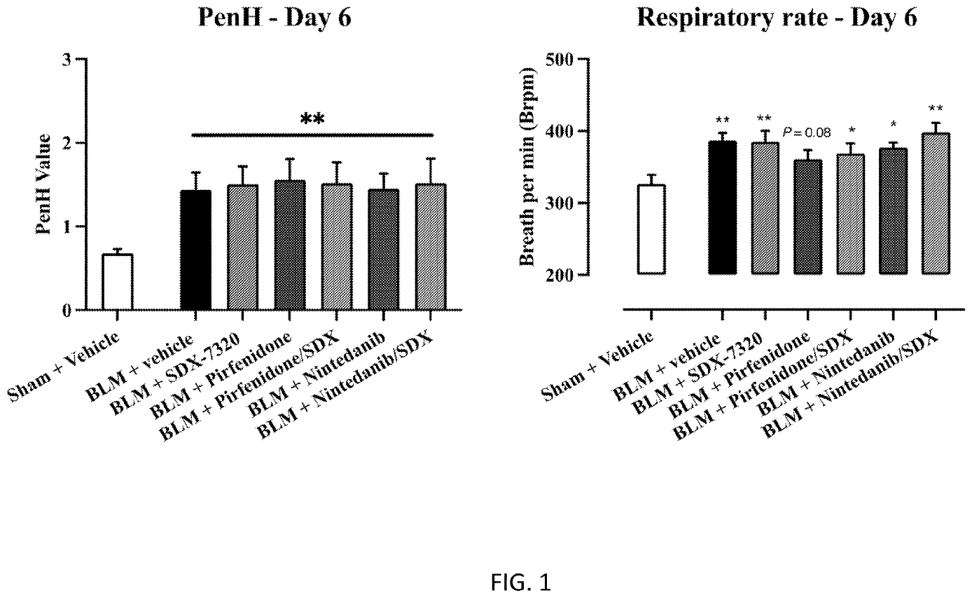

The following is a non-limiting example demonstrating the treatment of pulmonary fibrosis in an aged C57BL/6 male mouse model using Compound 1 (referred to herein and in FIGS. 1-12 and FIG. 22 as “SDX-7320” or “SDX”) and a combination of Compound 1 with either nintedanib (Nin), the current standard-of care therapy for patients with interstitial lung fibrosis, or pirfenidone.

Pulmonary fibrosis was modeled by treating the mice with bleomycin (BLM). As would be appreciated by the skilled artisan, BLM sulfate is a mixture of cytotoxic glycopeptide antibiotics isolated from Streptomyces verticillus and is used as an antineoplastic/antibiotic drug to treat various cancers. BLM causes the DNA in tumor cells to break, inducing apoptosis. BLM has been shown to have adverse effects on tissues such as the lung, and is associated with pulmonary side effects including inflammation and fibrosis. The most common route of administration BLM to induce pulmonary fibrosis is intratracheal, which generally causes an inflammatory response and increased epithelial apoptosis within the first 7 days, closely resembling acute lung injury (ALI). This is followed by 3 days of transitional period, in which inflammation resolves and the earliest fibrotic changes are detected. The fibrotic stage persists for a period of 3-4 weeks post-BLM and is characterized by excessive deposition of the extracellular matrix causing areas of fibrosis. As would be appreciated by the skilled artisan, the BLM-induced lung injury in mice is a well-documented pulmonary fibrosis model, and is particularly useful model for investigating the efficacy compounds in reducing pulmonary fibrosis and improving lung function post-injury.

Animals: Aged, male C57BL/6 mice from Jackson Laboratories, 72 weeks old at the time of randomization were used in this study.

Treatment: seven groups of mice were treated as put forth in Table A. Mice in groups 2-7 were challenged with bleomycin solution on Day 0 of the study by a single oropharyngeal installation of bleomycin solution such that the mice were given 1.5 U/kg based on the average body weight of the animals. On day 6 of the study, all mice were randomized based on body weight loss plus their PenH value (see below) as measured on Day 6.

Mice in treatment groups 1 and 2 were administered a vehicle control subcutaneously once every 4 days (Q4D) from day 7 to day 21. Mice in treatment group 3 were administered SDX-7320 subcutaneously Q4D from day 7 to day 21 at a dose of 8 mg/kg (i.e., 4 doses). Mice in treatment group 4 were administered pirfenidone orally (PO) twice a day (BID) from day 7 to day 21 at a dose of 100 mg/kg. Mice in treatment group 5 were administered nintedanib orally (PO) once a day (QD) from day 7 to day 21 at a dose of 50 mg/kg. Mice in treatment group 6 were administered SDX-7320 subcutaneously Q4D from day 7 to day 21 at a dose of 8 mg/kg and pirfenidone orally (PO) twice a day (BID) from day 7 to day 21 at a dose of 100 mg/kg. Mice in treatment group 7 were administered SDX-7320 subcutaneously Q4D from day 7 to day 21 at a dose of 8 mg/kg and nintedanib orally (PO) once a day (QD) from day 7 to day 21 at a dose of 50 mg/kg. Food and water were given ad libitum.

TABLE A

| | | | Treatment | Dose | |
|---|---|---|---|---|---|
| Treatment | # of | Challenge | (Day 7 | Route, | |
| Group | mice | (Day 0) | to 21) | Frequency | Dose |
| 1 | 10 | PBS | Vehicle | SC, Q4D | — |
| (“Sham”) | | | | | |
| 2 | 14 | Bleomycin | Vehicle | SC, Q4D | — |
| 3 | 14 | (1.5 U/kg | SDX-7320 | SC, Q4D | 8 mg/kg |
| 4 | 14 | oropha- | Pirfenidone | PO, BID | 100 mg/kg |
| 5 | 14 | ryngeal) | Nintedanib | PO, QD | 50 mg/kg |
| 6 | 14 | | SDX-7320 + | SC, Q4D + | 8 mg/kg + |
| | | | Pirfenidone | PO, BID | 100 mg/kg |
| 7 | 14 | | SDX-7320 + | SC, Q4D + | 8 mg/kg + |
| | | | Nintedanib | PO, QD | 50 mg/kg |

Observations and measurements: Daily observations of the behavioural and general health status of the animals were recorded. Body weight was also measured daily.

Respiratory Function: Respiratory function of the mice were measured using plethysmography. All mice were acclimated to the plethysmograph chamber environment. After the acclimatization period, the baseline functional respiratory parameters were measured by the whole-body plethysmograph (VivoFlow by SCIREQ) on Day 6 (before treatment introduction and process to the randomization), Days 13 and 20, post-BLM administration. Each measurement was performed with a mouse placed alone in an unrestrained whole-body plethysmography (WBP) chamber (Buxco system) to measure respiratory functions. The WBP trace provides specific information regarding the breathing patterns correlating to inflammation and fibrosis development. The functional respiratory parameters analyzed include respiratory rate and PenH (pulmonary congestion index). PenH, is used as an index of edema, inflammation or congestion (broncho-restriction). As would be appreciated by the skilled artisan, PenH is associated with pulmonary reactivity to injury. In lung fibrosis studies, PenH is used as an index representing the pulmonary state of the animals. If there is no elevation of the PenH value, the animal would have been unlikely to have developed the disease.

Hypoxia measurements: On Day 20 of the study, hypoxia-related parameters were evaluated using Abbott's Vet-scan i-STAT system. A single-use CG4+cartridge was used to evaluate blood gas measurement of partial Oxygen (pO₂), partial Carbon dioxide (pCO2), saturated Oxygen (SpO2), bicarbonate ions and lactate levels (indicating hypoxia) on arterial blood. Approximately 4-5 drops of arterial blood were collected from the tail to minimize impact blood homeostasis.

PV-Loop Assay: Prior to animal sacrifice on day 21 (post BLM administration), all mice were anesthetized with ketamine/xylazine, and the anesthesia was confirmed by the absence of reflex motion. Mice were tracheostomized with a 20 G stub needle cannula and ventilated with FlexiVent automated system from SCIREQ. Mice received an injection of vecuronium (1 mg/kg), a skeletal muscle relaxant, to avoid spontaneous breathing and were stabilized for at least 2 minutes. Following stabilization, all animals were ventilated with the Flexi Vent (SCIREQ). Default mouse ventilation parameter settings were submitted to Mouse Mechanics Scan script protocol, including SnapShot-150, Quickprime-3, PV Loop and Deep inflation ventilatory pattern protocols. All data acquisitions and compilations of lung functions were made via FlexiWare software V8 to determine phenotypic changes in lung parenchyma that occur with different lung pathologies. Lung compliance, or pulmonary compliance, is a measure of the lung's ability to stretch and expand (distensibility of elastic tissue) i.e., lung compliance reflecting the change in volume for any given applied pressure. As would be appreciated by the skilled artisan, low compliance indicates a stiff lung (one with highly elastic recoil), which is often observed in fibrosis. High compliance indicates a pliable lung (one with low elastic recoil), and is often observed in emphysema.

Lung weight: Lungs were harvested, and the wet lung weights (signifying edema) were recorded.

Histopathological Preparation: The whole lung of each mouse was harvested, weighed, flushed with 0.9% NaCl and inflated with 10% neutral buffered formalin solution (NBF). The left lobe was kept in fixative for 48 h and was sent to the Histopathology Laboratory Althisia (Troyes, France) to make slides for the histopathological analysis. For each animal, three sections (5-μm thick) were cut (100 μm apart), laid on one slide and stained with picrosirius red (PSR). Alternatively, the sections were labeled with an ab270993 antibody (rabbit monoclonal antibody to Collagen I). After fixation and sectioning, slides labeled with the ab270993 first underwent EDTA based epitope revival and were blocked with serum for 20 minutes before being incubated with the primary ab270993antibody (concentration, 1/1000) for 1 hour and subsequently incubated with the secondary antibody for 45 minutes. All slides were scanned using the NanoZoomer®-SQ scanner (Hamamatsu), at the magnification of X20 (0.452 μm/pixel) and digital slides of whole sections were captured using NDP view 2 Hamamatsu software. Images were then processed and automated analysis of the pulmonary fibrosis (density of parenchymal tissue, percentage of fibrotic foci, collagen percentage content) was performed.

Histopathological Analysis: Automatic quantification of morphological features of pulmonary fibrosis were performed using MorphoQuant™ (Biocellvia, France), an automated AI-based digital pathology software for quantification, from the digitalized images (scans) of PSR-stained or antibody labeled lung sections. MorphoQuant™ uses principles of morphometric analysis to detect and quantify specific lung disease features from histological slides (Gilhodes et al. 2017; Michaudel, Fauconnier, et al. 2018). The following morphological endpoints were quantitatively assessed:
a) Parenchyma tissue density (expressed as “Tissue density (%)”), PSR-stained: Tissue density is the ratio of the area occupied by the lung tissue to the whole section area, expressed in percent. Lumens from alveoli, bronchi, bronchioles and blood vessels are not considered.
b) High-density pulmonary tissue (expressed as “Fibrotic foci (%)”), PSR-stained: Fibrotic foci correspond to fibrous thickening of alveolar parenchyma and are therefore characterized throughout the lung section by specific high-density values. Fibrotic foci are expressed as the percent ratio of area occupied by high-density lung tissue of a section to the whole section area.
c) Total collagen (expressed as “Collagen (%)”), PSR-stained: Total collagen corresponds to the ratio of the area occupied by collagen to the whole section area, expressed in percent.
d) Collagen, type 1, alpha 1 (expressed as “COL1A1 area (%)”), ab270993 antibody labeled: Collagen, type 1, alpha 1 corresponds to the ratio of the area occupied by collagen, type 1, alpha 1 to the whole section area, expressed in percent.

Statistical Analysis: All parameters were analyzed in the same manner and results were expressed as means±SEM with Graph Pad Prism Software version 8.0 (San Diego, CA, USA). The data homoscedasticity was tested with Bartlett's test. ANOVA was performed, followed by a post-hoc test (Uncorrected Fisher's LSD). In the case where distribution was not normalized, a nonparametric analysis (Kruskal-Wallis) followed by an uncorrected Dunn's test was performed. Statistical comparisons between sham and BLM/vehicle were conducted, followed by comparisons between BLM/vehicle and treated groups. Differences were considered statistically significant when P values were less than 0.05. * Shown difference versus Sham animals, #shown difference versus BLM+Vehicle group, $ shown difference versus BLM+Nintedanib. * Means P<0.05, **P<0.01, ***P<0.001; #means P<0.05, ##P<0.01, ###P<0.001, while $ Means P<0.05, $$ P<0.01 and $$$ P<0.001.

Results: FIG. 1 shows the PenH value (left panel) and respiratory rate (right panel) of the mice in each treatment group on Day 6 of the study following randomization of the mice into the different treatment groups. As would be appreciated by the skilled artisan, randomization allows for relative equal distribution of each group in terms of the severity of the disease prior to treatment initiation.

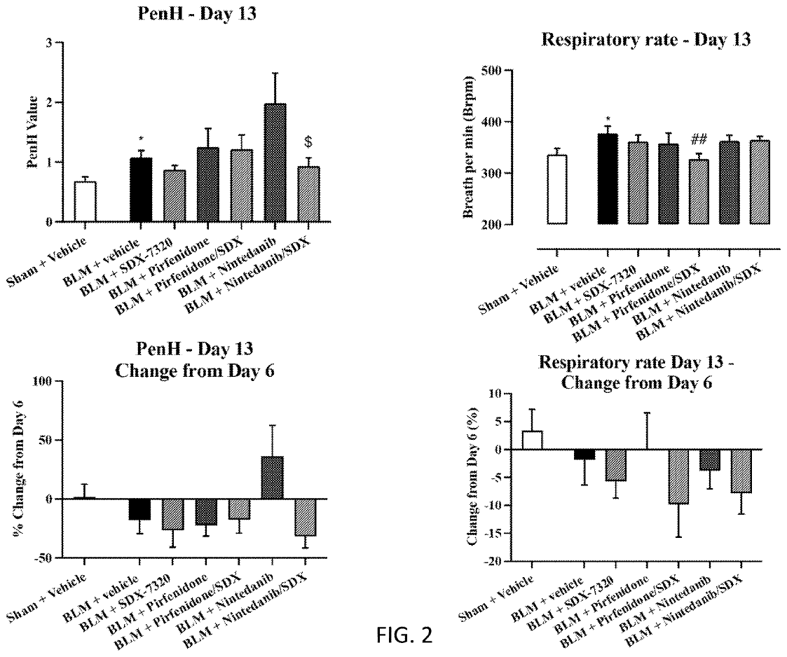

FIG. 2 shows the PenH value on day 13 of the study (top left panel), the change in PenH value from day 6 to day 13 of the study (bottom left panel), the respiratory rate on day 13 of the study (top right panel) and the change in respiratory rate from day 6 to day 13 of the study (bottom rate panel) in each treatment group.

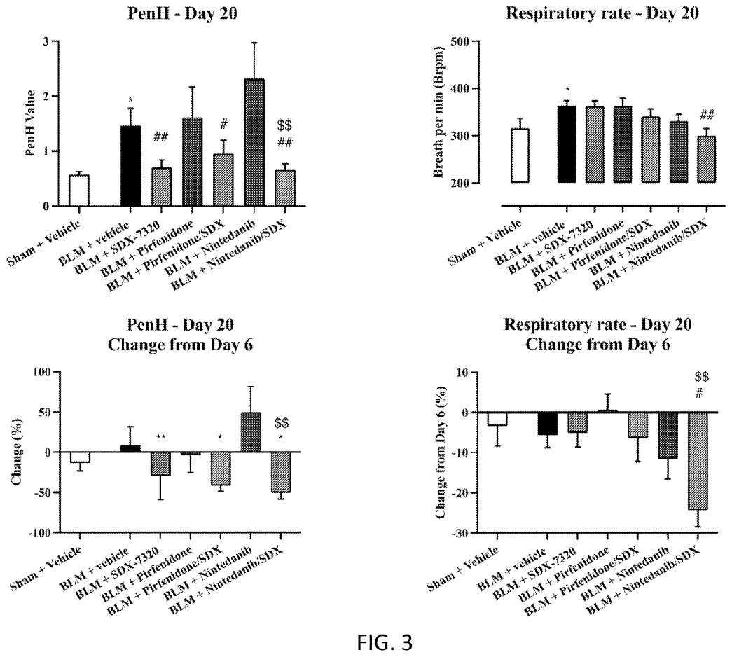

FIG. 3 shows the PenH value on day 20 of the study (top left panel), the change in PenH value from day 6 to day 20 of the study (bottom left panel), the respiratory rate on day 20 of the study (top right panel) and the change in respiratory rate from day 6 to day 20 of the study (bottom rate panel) in each treatment group. As shown in FIG. 3, after 20 days following BLM administration, the pulmonary function reductions were maintained in the BLM-treated group compared to the Sham group. As observed on days 6 and 13, BLM administration caused an increase in pulmonary congestion (as indicated by PenH values) in BLM treated animals. Nintedanib alone increased the pulmonary congestion while SDX-7320 monotherapy reduced pulmonary congestion, and treatment with a combination of SDX-7320 and nintedanib or a combination of SDX-7320 and pirfenidone reduced congestion/inflammation. Moreover, as shown in FIG. 3, the average respiratory rate was significantly greater in the BLM-treated group. Treatment with a combination of SDX-7320 and nintedanib or a combination of SDX-7320 and pirfenidone reduced respiratory rates.

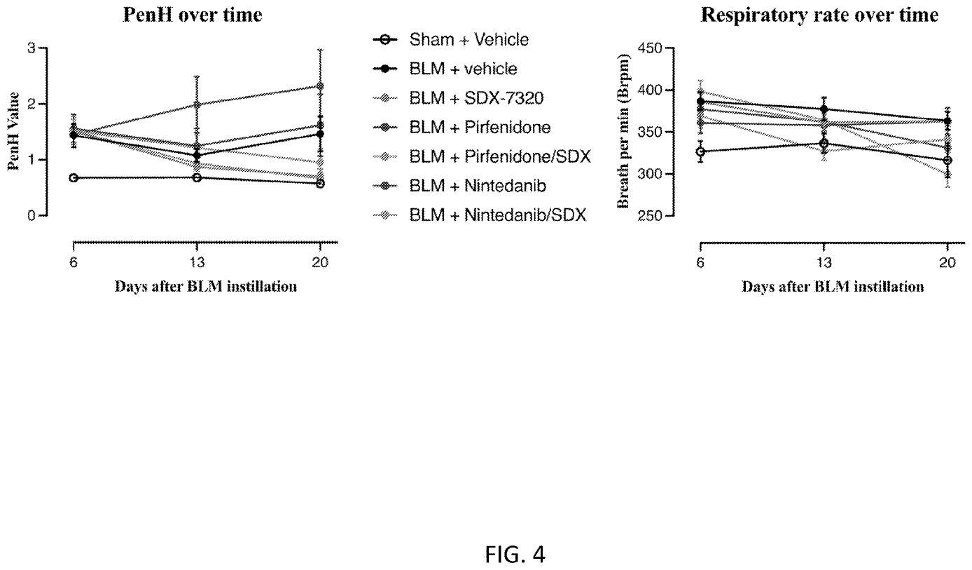

FIG. 4 shows the PenH values (left panel) and respiratory rates (right panel) in each of the treatment groups over the course of the study. As shown in FIG. 4, the improvements in the condition of the mice treated with SDX-7320 monotherapy, a combination of SDX-7320 and nintedanib, or a combination of SDX-7320 and pirfenidone appeared one week after treatment. As shown in FIG. 4, 21 days following BLM injury, and fourteen days after the beginning of treatment, mice treated either with SDX-7320 monotherapy, a combination of SDX-7320 and nintedanib, or a combination of SDX-7320 and pirfenidone saw improvements in their condition.

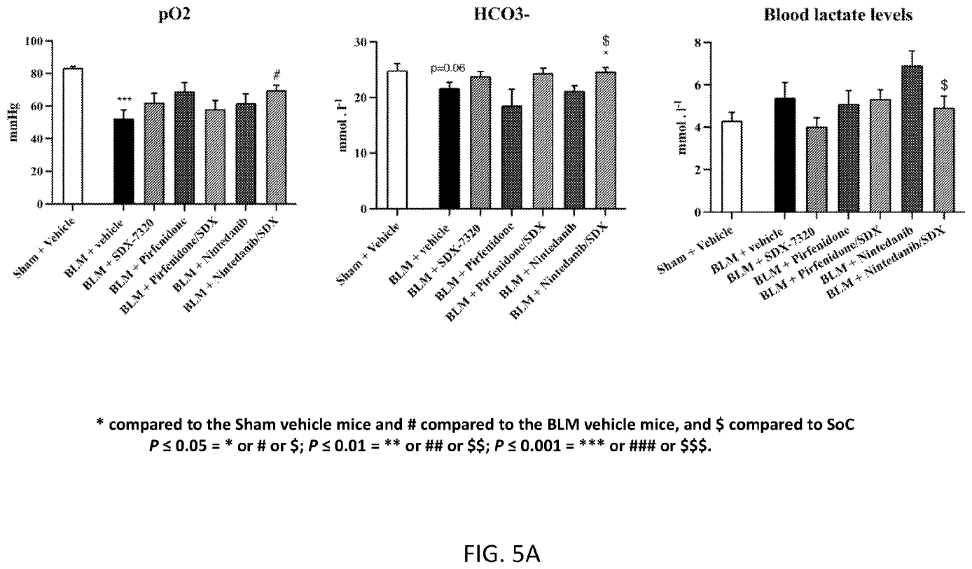

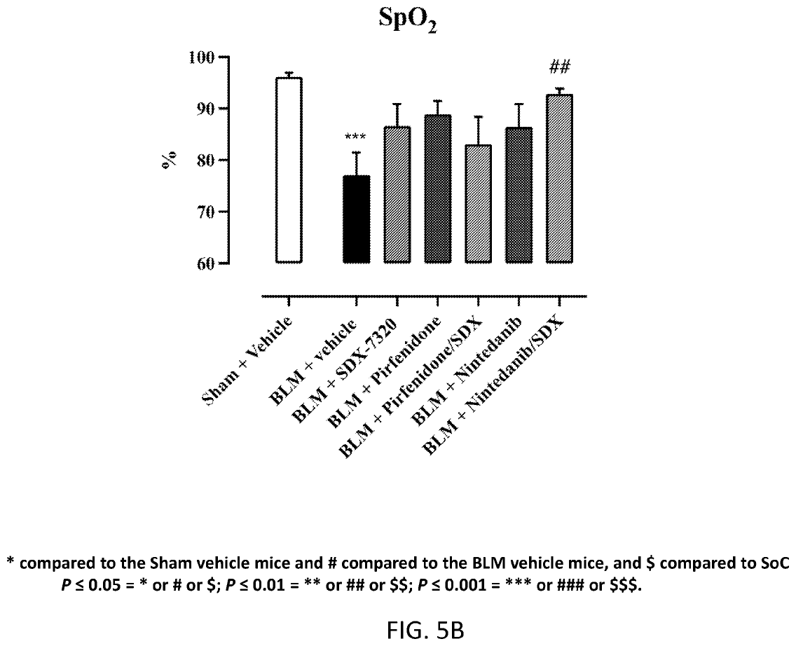

On day 21 of the study, the respiratory capacity was evaluated by measuring key blood/gas exchange parameters, including arterial blood saturation (SpO₂), oxygen pressure in blood (pO₂), blood lactate levels, and blood bicarbonate (HCO3−) ion levels. The oxygen pressure in blood (pO₂), blood bicarbonate (HCO3−) ion levels and blood lactate levels are shown in FIG. 5A (left panel to right panel, respectively), and the SpO₂ values are shown in FIG. 5B. As would be appreciated by the skilled artisan, in pulmonary fibrosis, the arterial saturation is considered a key parameter describing the pulmonary status. As shown in FIG. 5B, the SpO₂ values were significantly lower in BLM-treated animals compared to the Sham animals providing further evidence of lung function damage. As shown in FIG. 5B, treatment with either SDX-7320 alone, or in combination with pirfenidone or nintedanib improved SpO₂ values. This improvement further corroborated by improvements in the pO₂ levels, which are shown in the left panel of FIG. 5A.

As would be appreciated by the skilled artisan, one of the physiological impacts of hypoxia is whole-body anaerobic metabolism leading to increases in blood lactate levels. As shown in the right panel of FIG. 5A, BLM-treated mice had higher blood lactate level compared to Sham mice. In direct correlation with the SpO₂ and pO₂, SDX-7320 treatment tended to decrease this level. Treatment with either SDX-7320 alone, or in combination with pirfenidone or nintedanib resulted in a decrease in blood lactate levels. These improvements are further supported by and correlated with the blood bicarbonate ion levels, as shown in the middle panel of FIG. 5A.

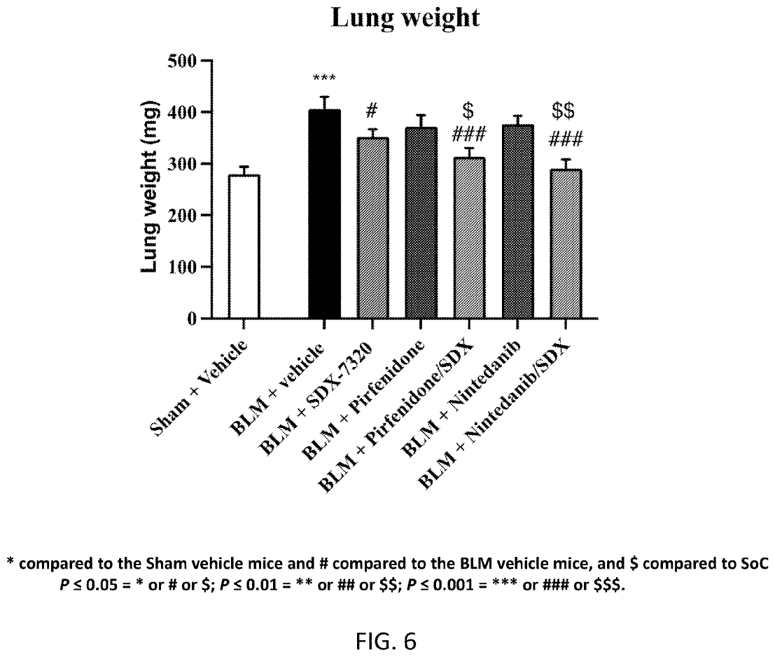

As would be appreciated by the skilled artisan, edema is a hallmark of lung inflammation, and is associated with vascular leakiness and extracellular matrix deposition, which can increase lung weight via interstitial fluid accumulation and collagen deposition. After sacrifice of the mice after the 21 day study, the lungs were harvested, carefully sponged and weighed. Lung edema was evaluated by measuring the wet lung weight, and the results are shown in FIG. 6. As shown in FIG. 6, BLM injury induced a significant increase in the lung weights. Treatment with SDX-7320 alone, or in combination with pirfenidone or nintedanib, decreased the lung weight compared to BLM-treated mice. Nintedanib or pirfenidone alone did not significantly reduce lung weight.

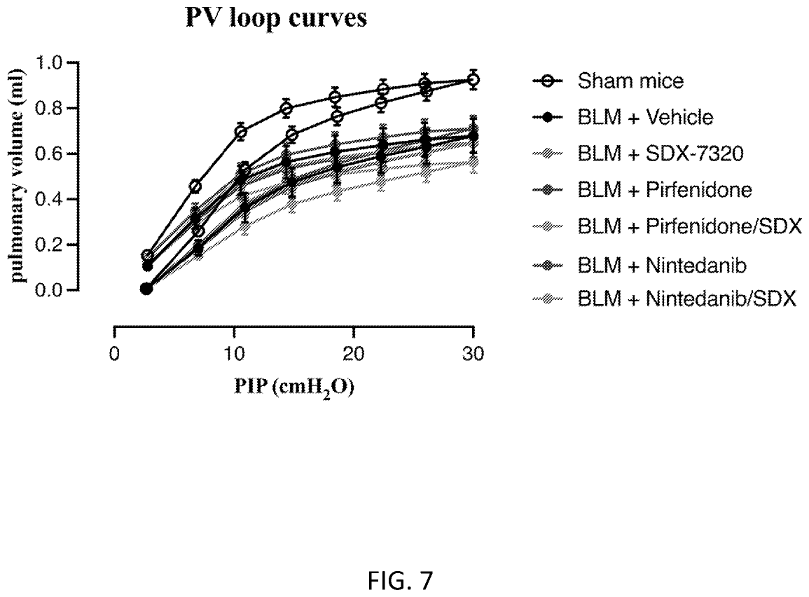

Immediately before sacrifice on the terminal surgery day, mice underwent a series of tests using a FlexiVent automated system (SCIREQ) to evaluate different parameters of the lung functions. Pressure-Volume curves (PV-loop) were assessed on all mice using a series of automated predefined pressure steps from 0 to 30 cmH₂O, and analysis is shown in FIG. 7.

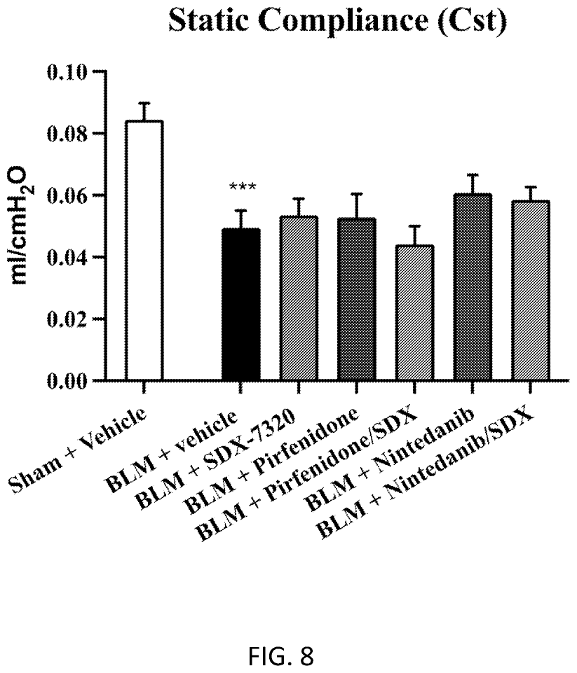

Static compliance (Cst) was also measured in each treatment group, and the analysis is shown in FIG. 8.

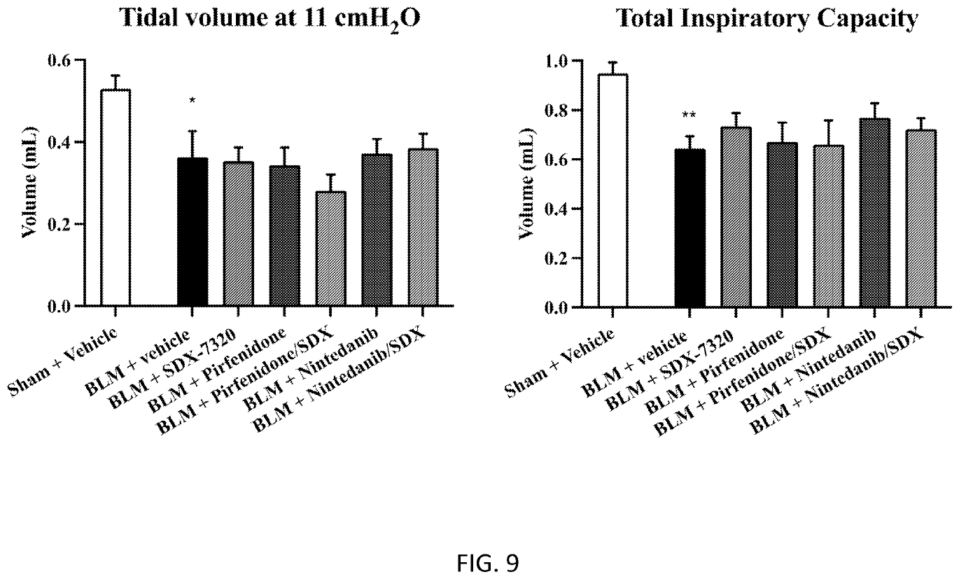

Inflation volume (tidal volume) and pulmonary capacity (total inspiratory capacity) were also measured in each treatment group, and the analysis is shown in FIG. 9.

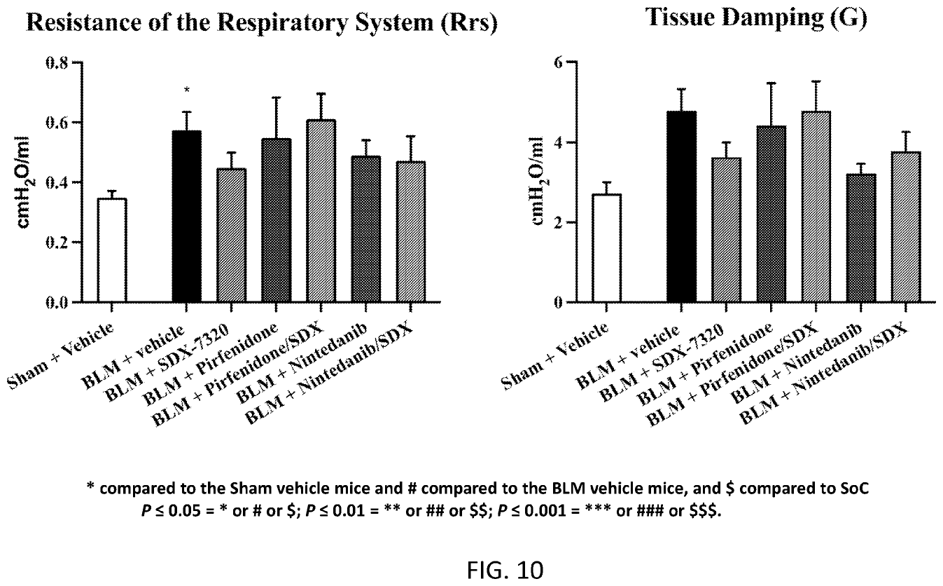

Resistance of the Respiratory System (Rrs) and Tissue Damping (G) were also measured in each treatment group, and the analysis is shown in FIG. 10. As would be appreciated by the skilled artisan, tissue damping is related to alveoli resistance and mainly responsible for the whole pulmonary system resistance. As shown in FIG. 10, tissue resistance was significantly increased in the BLM+vehicle mice. Treatment with either SDX-7320 alone, or in combination with pirfenidone or nintedanib, improved this alveoli resistance to pressure variation.

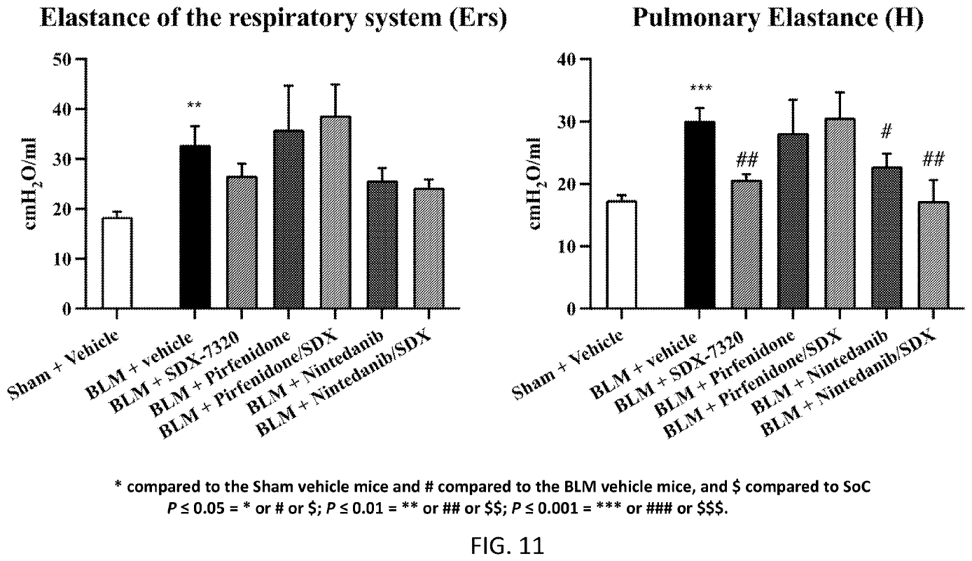

Elastance of the respiratory system (Ers) and pulmonary elastance (H) were also measured in each treatment group, and the analysis is shown in FIG. 11. As would be appreciated by the skilled artisan, Pulmonary elastance (H) which represents the elastic energy stored within the tissues following the imposed deformation and therefore the ability of the tissue to retract and revert to its original shape. It is an index of tissue stiffness. As shown in FIG. 11, tissue stiffness was increased in the BLM mice and was improved by treatment with SDX-7320, with or without the addition of nintedanib.

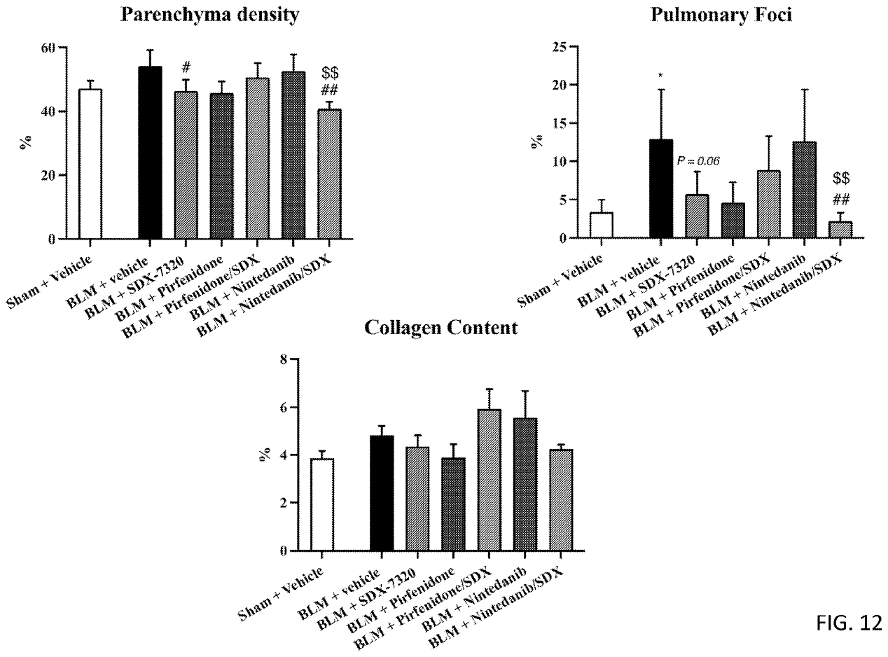

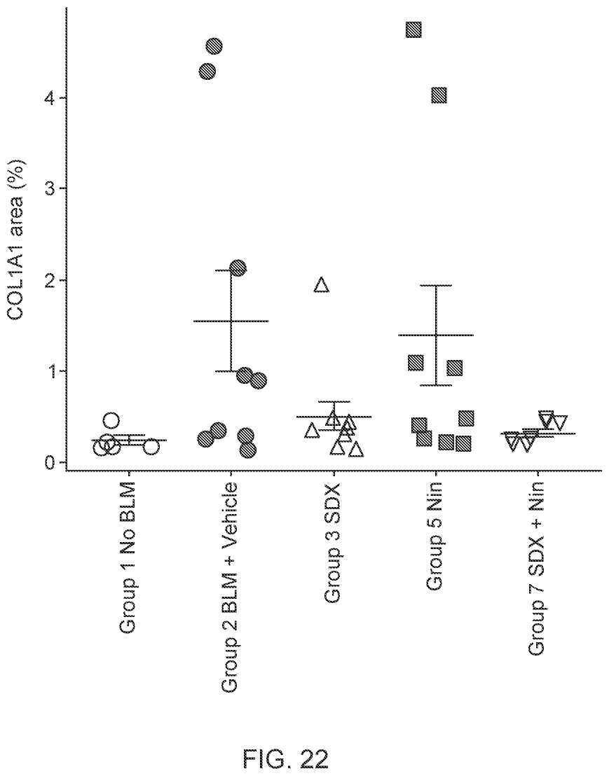

Automated histopathological analysis of lung samples was performed to determine Parenchyma tissue density, Fibrotic foci (pulmonary foci), and total collagen, as described above. The results of this analysis are shown in FIG. 12 (Parenchyma tissue density, Fibrotic foci, and total collagen), FIG. 22 (Collagen, type 1, alpha 1) and corresponding Table C.

TABLE C

| Treatment | # of | Challenge | Treatment | Dose Route, | | COL1A1 |
|---|---|---|---|---|---|---|
| Group | mice | (Day 0) | (Day 7 to 21) | Frequency | Dose | area (%) |
| Group 1 | 5 | PBS | Vehicle | SC, Q4D | — | 0.241 ± 0.115 |
| Group 2 | 9 | Bleomycin | Vehicle | SC, Q4D | — | 1.54 ± 1.641 |
| Group 3 | 10 | (1.5 U/kg | SDX | SC, Q4D | 8 mg/kg | 0.501 ± 0.494 |
| Group 5 | 9 | oropharyngeal) | Nin | PO, QD | 50 mg/kg | 1.387 ± 1.638 |
| Group 7 | 8 | | SDX + Nin | SC, Q4D + | 8 mg/kg + | 0.319 ± 0.106 |
| | | | | PO, QD | 50 mg/kg | |

As shown in FIG. 12, treatment with SDX-7320 alone, or in combination with nintedanib or pirfenidone improved both pulmonary foci, parenchyma density and collagen content.

As shown in FIG. 22 and corresponding Table C, treatment with SDX-7320 alone or in combination with nintedanib greatly inhibits the deposition of collagen in lung tissue following exposure to bleomycin. SDX-7320 in combination with nintedanib further improved the response of SDX-7320, with nearly full inhibition of collagen deposition. In contrast, nintedanib alone had almost no activity with regards to collagen deposition.

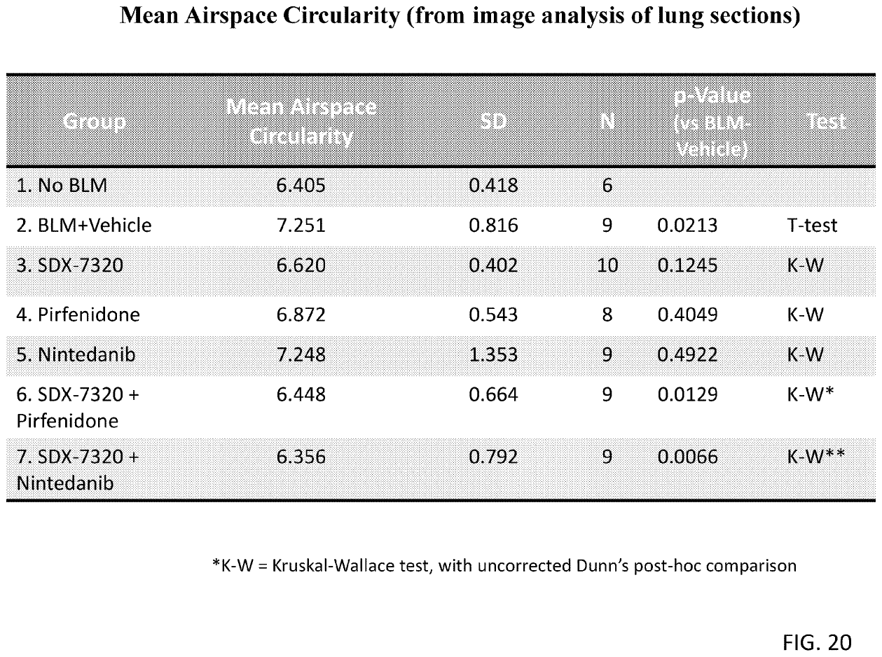

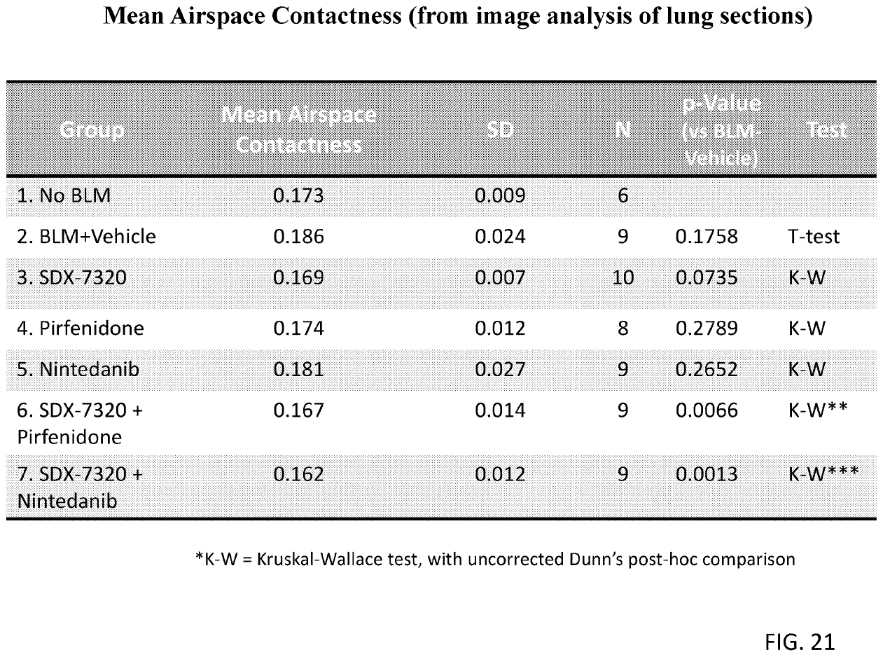

Finally, automated histopathological analysis of lung samples was performed to determine mean airspace circularity and mean airspace contactness in each of the treatment groups. The results of this analysis are summarized in FIG. 20 and FIG. 21, respectively. As shown in FIG. 20 and FIG. 21, differences were observed the mice treated with the vehicle control as compared to mice treated with a combination of SDX-7320 and pirfenidone or a combination of SDX-7320 and nintedanib.

Taken together, these results demonstrate that SDX-7320, and combinations of SDX-7320 with either nintedanib or pirfenidone can be used to effectively treat pulmonary fibrosis and to improve lung function after the inducement of pulmonary fibrosis.

### Example 2—Treatment of Pulmonary Fibrosis Using Compound 1 and Combinations of Compound 1 and Nintedanib

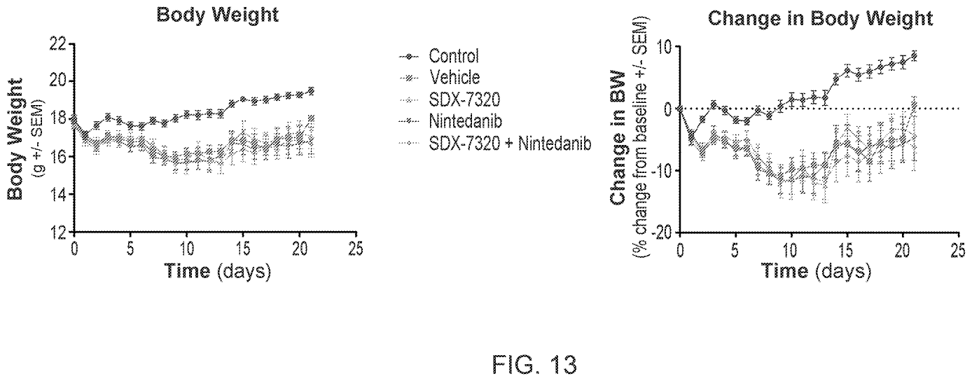

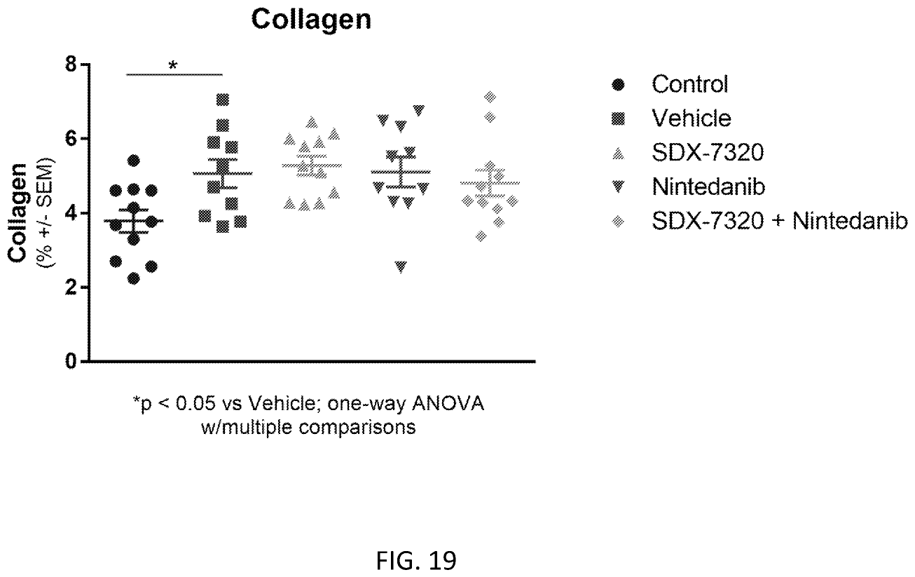

The following is a non-limiting example demonstrating the treatment of pulmonary fibrosis in an 8 week-old female C57BL/6 mouse model using Compound 1 (referred to herein and in FIGS. 13-19 as “SDX-7320” or “SDX”) and a combination of Compound 1 with nintedanib.

Pulmonary fibrosis was modeled by treating the mice with bleomycin (BLM). To induce lung fibrosis, a single dose of bleomycin (3 mg/kg in a volume of 50 μl) was instilled intra-tracheally on day zero of the study.

Treatment: five groups of mice were treated as put forth in Table B. Mice in groups 2-5 were challenged with bleomycin solution on Day 0 of the study, as described above. The mice were randomized into different treatment groups on Day 7 of the study based on change in body weight from baseline.

Mice in treatment group 2 were administered a vehicle control subcutaneously once every 4 days (Q4D) from day 7 to day 20. Mice in treatment group 3 were administered SDX-7320 subcutaneously Q4D from day 7 to day 21 at a dose of 8 mg/kg. Mice in treatment group 4 were administered nintedanib orally (PO) once a day (QD) from day 7 to day 20 at a dose of 100 mg/kg. Mice in treatment group 5 were administered SDX-7320 subcutaneously Q4D from day 7 to day 20 at a dose of 8 mg/kg and nintedanib orally (PO) once a day (QD) from day 7 to day 21 at a dose of 100 mg/kg. All mice were sacrificed on Day 21 of the study.

TABLE B

| | | | Treatment | Dose | |
|---|---|---|---|---|---|
| Treatment | # of | Challenge | (Day 7 | Route, | |
| Group | mice | (Day 0) | to 21) | Frequency | Dose |
| 1 | 12 | — | — | SC, Q4D | — |
| (“Sham”) | | | | | |
| 2 | 12 | Bleomycin | Vehicle | SC, Q4D | — |
| 3 | 12 | (3 mg/kg) | SDX-7320 | SC, Q4D | 8 mg/kg |
| 4 | 12 | | Nintedanib | PO, QD | 100 mg/kg |
| 5 | 12 | | SDX-7320 + | SC, Q4D + | 8 mg/kg + |
| | | | Nintedanib | PO, QD | 100 mg/kg |

Results: FIG. 13 shows the body weight and change in body weight in each of the treatment groups over the course of the study.

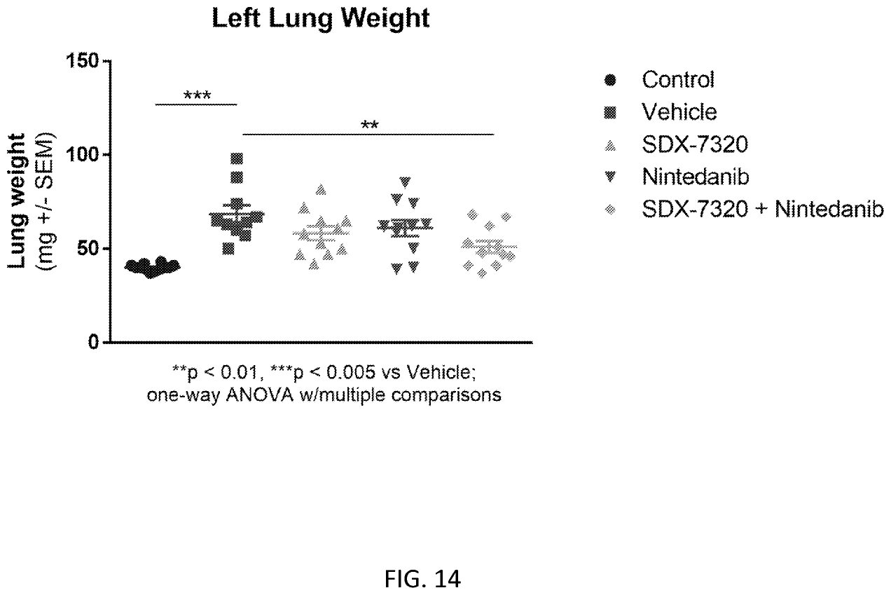

FIG. 14 shows the weight of the left lung at the conclusion of the study in each of the treatment groups. As shown in FIG. 14, mice treated with a combination of SDX-7320 and nintedanib showed a decreased in mean left lung weight as compared to the vehicle control group. Furthermore, mean left lung weight in mice treated with SDX-7320 alone also tended to decrease as compared to the vehicle control group.

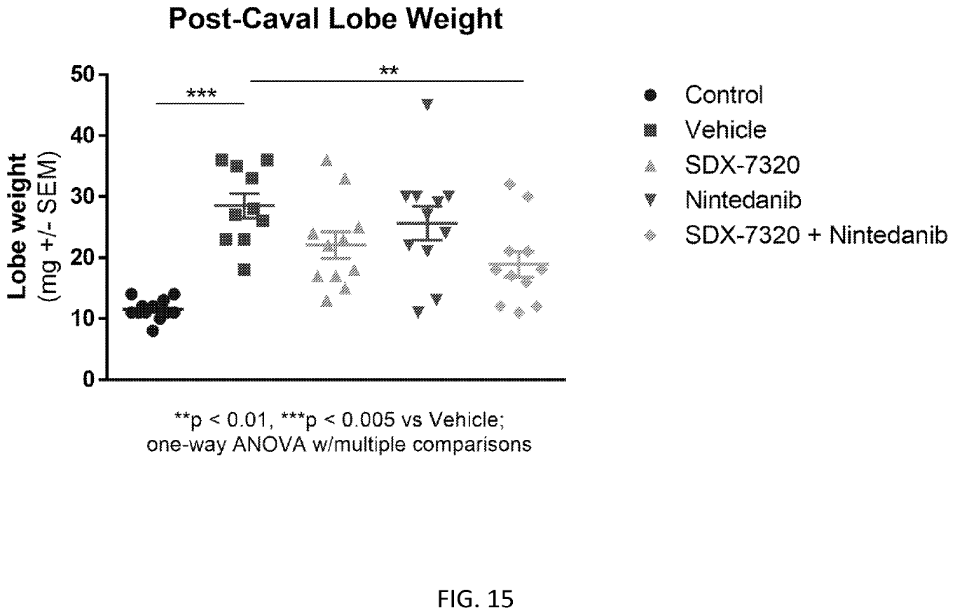

FIG. 15 shows the post-caval lobe weight at the conclusion of the study in each of the treatment groups. As shown in FIG. 15, mice treated with a combination of SDX-7320 and nintedanib showed a decreased in post-caval lobe weight as compared to the vehicle control group. Furthermore, post-caval lobe weight in mice treated with SDX-7320 alone also tended to decrease as compared to the vehicle control group.

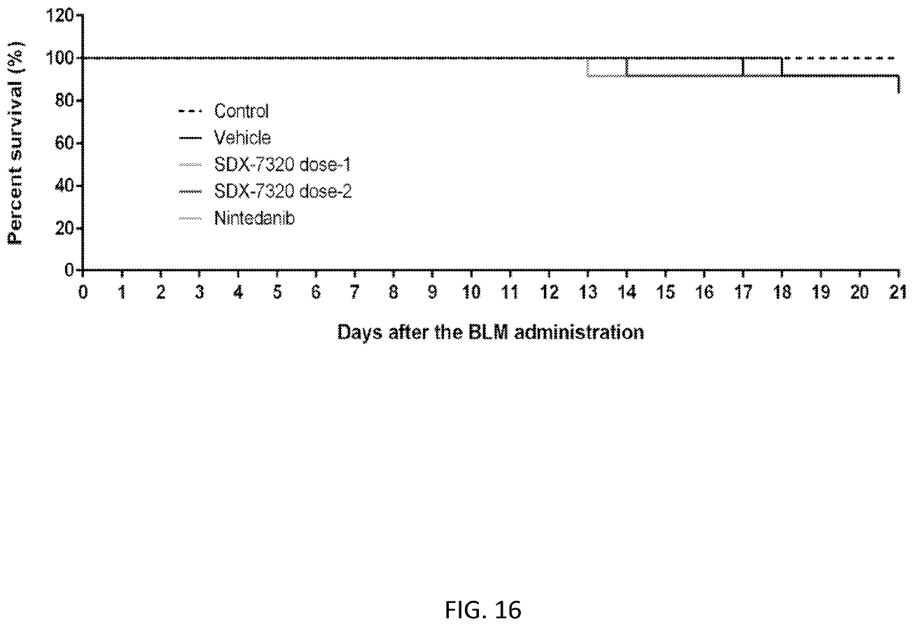

FIG. 16 shows the survival of the mice in each of the control groups over the course of the study.

Additionally, automated histopathological analysis was performed on lung samples obtained from the mice at the conclusion of the study. Briefly, formalin-fixed paraffin-embedded blocks were processed. For each mouse, 1 series of 3 sections (5-μm thick) were cut and spaced by 50 μm. As each block contained 3 lobes, 9 sections were laid on a slide, and stained with PSR. Digital slides of whole sections were captured using NDP.view 2 Hamamatsu software at the magnification of X20. Automatic quantification of morphological features of PF in the lung was performed using Biocellvia's imaging assay MorphoQuant®-Lung from digitalized images (scans) of PSR-stained lung sections. The following endpoints were quantitatively assessed, as described in Example 1:
a) tissue density (%)
b) fibrotic foci (%)
c) collagen (%).

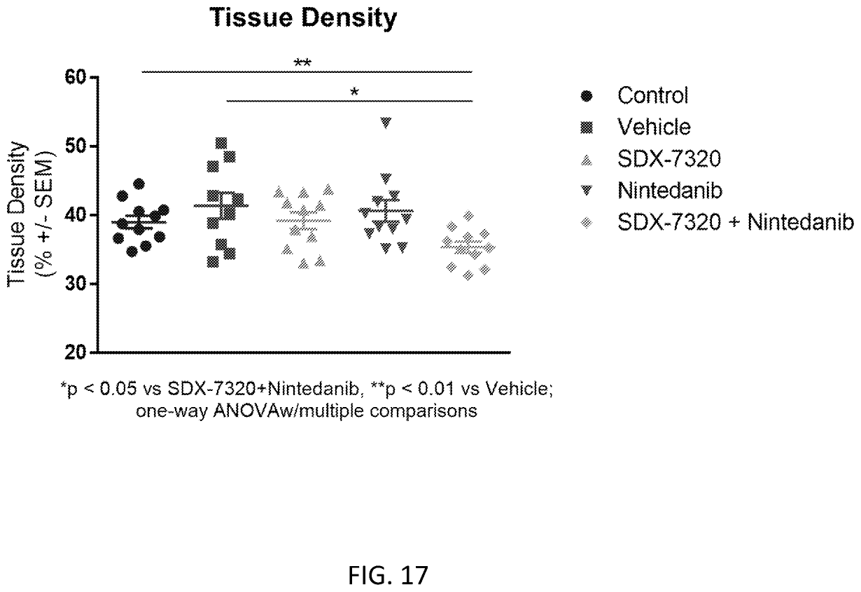

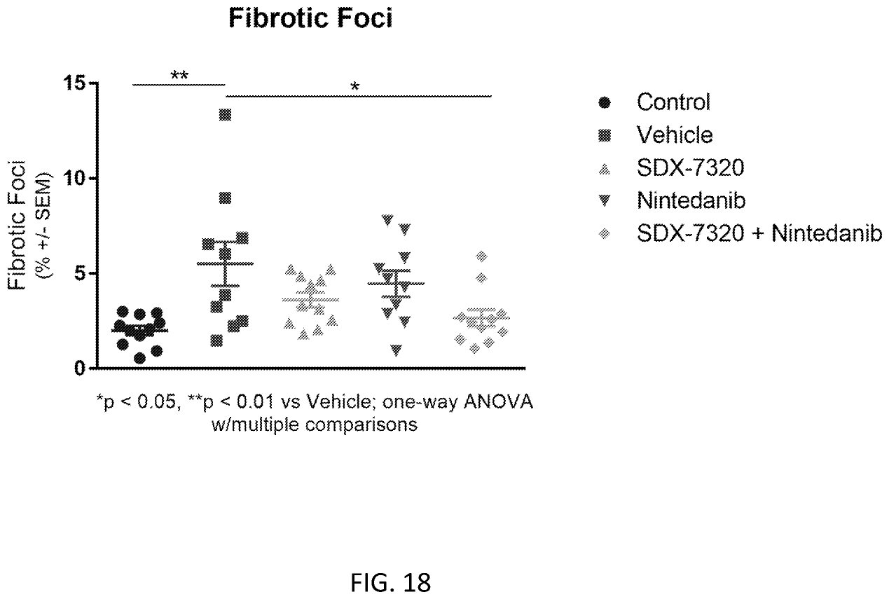

The results of the tissue density, fibrotic foci and collagen analyses are shown in FIGS. 17-19, respectively. Tissue density and collagen content was reduced in the combination groups relative to the vehicle and each treatment alone. As shown in FIG. 18, treatment with a combination of SDX-7320 and nintedanib resulted in a decrease in fibrotic foci as compared to both the vehicle control group and treatment with nintedanib alone.

Taken together, these results demonstrate that SDX-7320, and combinations of SDX-7320 with either nintedanib or pirfenidone can be used to effectively treat pulmonary fibrosis and to improve lung function after the inducement of pulmonary fibrosis.

What is claimed is:

1. A combination comprising at least one MetAP2 inhibitor, or a pharmaceutically acceptable salt thereof, and nintedanib, or a pharmaceutically acceptable salt thereof, for use in treating pulmonary fibrosis in a subject.

2. A method of treating pulmonary fibrosis in a subject in need thereof, the method comprising administering to the subject at least one MetAP2 inhibitor, or a pharmaceutically acceptable salt thereof, and nintedanib, or a pharmaceutically acceptable salt thereof.

3. A MetAP2 inhibitor, or a pharmaceutically acceptable salt thereof, for use in a method of treating pulmonary fibrosis in a subject, wherein the method further comprises administration of nintedanib, or a pharmaceutically acceptable salt thereof.

4. Nintedanib, or a pharmaceutically acceptable salt thereof, for use in a method of treating pulmonary fibrosis in a subject, wherein the method further comprises administration of at least one MetAP2 inhibitor or a pharmaceutically acceptable salt thereof.

5. The combination for use of claim 1, the method of claim 2, the MetAP2 inhibitor for use of claim 3, or the nintedanib for use of claim 4, wherein the at least one MetAP2 inhibitor, or pharmaceutically acceptable salt thereof, and the nintedanib, or pharmaceutically acceptable salt thereof, are administered concurrently or in temporal proximity.

6. The combination for use of claim 1 or 5, the method of claim 2 or 5, the MetAP2 inhibitor for use of claim 3 or 5, or the nintedanib for use of claim 4 or 5, wherein the MetAP2 inhibitor is selected from: 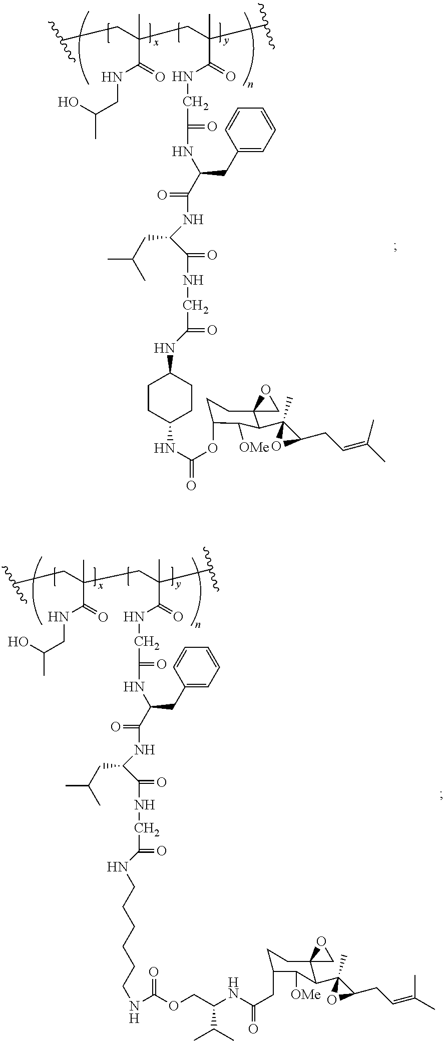 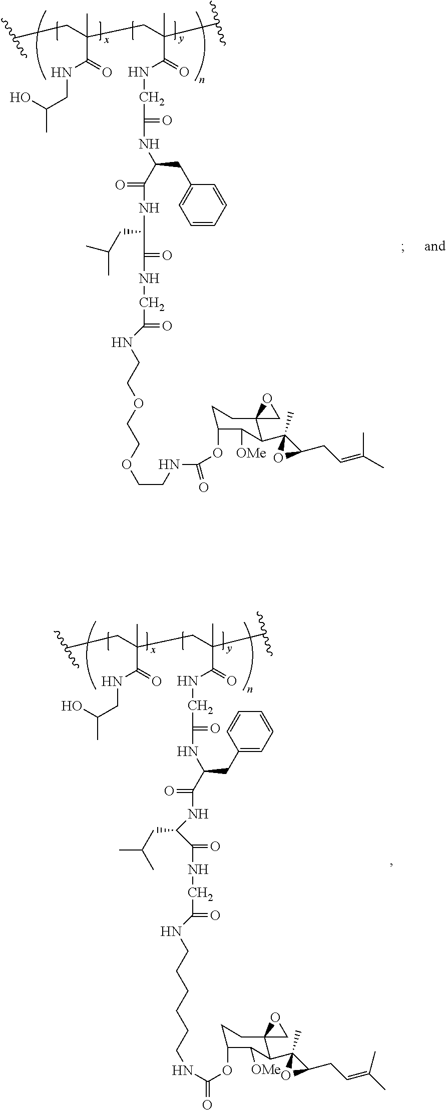
or a pharmaceutically acceptable salt thereof,
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

7. The combination for use of claim 1 or 5-6, the method of claim 2 or 5-6, the MetAP2 inhibitor for use of claim 3 or 5-6, or the nintedanib for use of claim 4 or 5-6, wherein the MetAP2 inhibitor is: 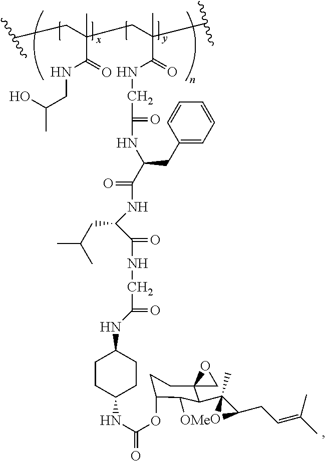
or a pharmaceutically acceptable salt thereof, wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

8. The combination for use of claim 1 or 5-7, the method of claim 2 or 5-7, the MetAP2 inhibitor for use of claim 3 or 5-7, or the nintedanib for use of claim 4 or 5-7, wherein the nintedanib is nintedanib esylate.

9. The combination for use of claim 1 or 5-8, the method of claim 2 or 5-8, the MetAP2 inhibitor for use of claim 3 or 5-8, or the nintedanib for use of claim 4 or 5-8, wherein pulmonary fibrosis is Asbestosis, COVID-19-related pulmonary fibrosis, Drug-induced pulmonary fibrosis, Hypersensitivity Pneumonitis (HP), Idiopathic pulmonary fibrosis (IPF), Idiopathic Non-specific interstitial pneumonia (NSIP), Pneumoconiosis, Rheumatoid Arthritis Interstitial Lung Disease (RA-ILD), Sarcoidosis, Silicosis, Systemic sclerosis, or treatment-induced pulmonary fibrosis, preferably wherein the pulmonary fibrosis is IPF or treatment-induced pulmonary fibrosis.

10. A combination comprising at least one MetAP2 inhibitor, or a pharmaceutically acceptable salt thereof, and pirfenidone, or a pharmaceutically acceptable salt thereof, for use in treating pulmonary fibrosis in a subject.

11. A method of treating pulmonary fibrosis in a subject in need thereof, the method comprising administering to the subject at least one MetAP2 inhibitor, or a pharmaceutically acceptable salt thereof, and pirfenidone, or a pharmaceutically acceptable salt thereof.

12. A MetAP2 inhibitor, or a pharmaceutically acceptable salt thereof, for use in a method of treating pulmonary fibrosis in a subject, wherein the method further comprises administration of pirfenidone, or a pharmaceutically acceptable salt thereof.

13. Pirfenidone, or a pharmaceutically acceptable salt thereof, for use in a method of treating pulmonary fibrosis in a subject, wherein the method further comprises administration of at least one MetAP2 inhibitor or a pharmaceutically acceptable salt thereof.

14. The combination for use of claim 10, the method of claim 11, the MetAP2 inhibitor for use of claim 12, or the pirfenidone for use of claim 13, wherein the at least one MetAP2 inhibitor, or pharmaceutically acceptable salt thereof, and the pirfenidone, or pharmaceutically acceptable salt thereof, are administered concurrently or in temporal proximity.

15. The combination for use of claim 10 or 14, the method of claim 11 or 14, the MetAP2 inhibitor for use of claim 12 or 14, or the pirfenidone for use of claim 13 or 14, wherein the MetAP2 inhibitor is selected from: 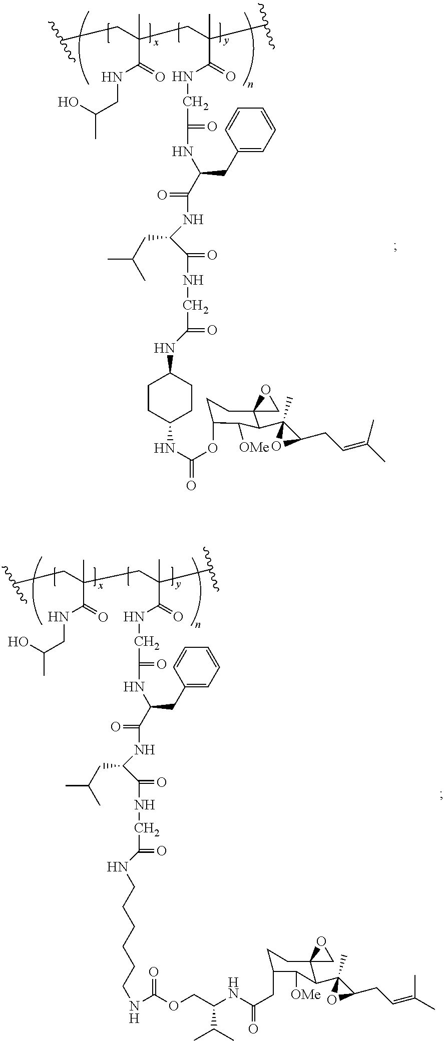 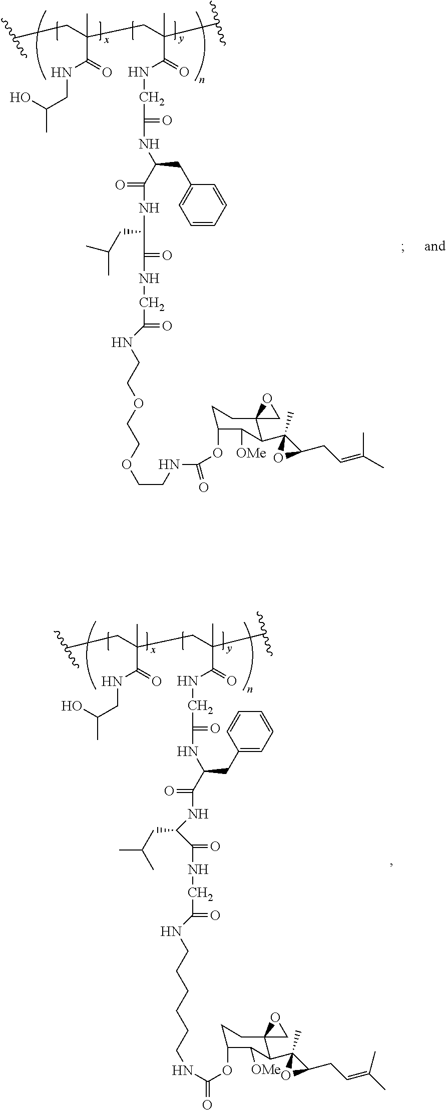
or a pharmaceutically acceptable salt thereof,
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

16. The combination for use of claim 10 or 14-15, the method of claim 11 or 14-15, the MetAP2 inhibitor for use of claim 12 or 14-15, or the pirfenidone for use of claim 13 or 14-15, wherein the MetAP2 inhibitor is: 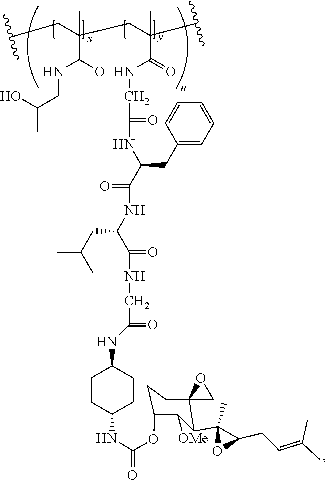
or a pharmaceutically acceptable salt thereof, wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

17. The combination for use of claim 10 or 14-16, the method of claim 11 or 14-16, the MetAP2 inhibitor for use of claim 12 or 14-16, or the pirfenidone for use of claim 13 or 14-16, wherein pulmonary fibrosis is Asbestosis, COVID-19-related pulmonary fibrosis, Drug-induced pulmonary fibrosis, Hypersensitivity Pneumonitis (HP), Idiopathic pulmonary fibrosis (IPF), Idiopathic Non-specific interstitial pneumonia (NSIP), Pneumoconiosis, Rheumatoid Arthritis Interstitial Lung Disease (RA-ILD), Sarcoidosis, Silicosis, Systemic sclerosis or treatment-induced pulmonary fibrosis, preferably wherein the pulmonary fibrosis is IPF or treatment-induced pulmonary fibrosis.

18. A combination comprising at least one MetAP2 inhibitor, or a pharmaceutically acceptable salt thereof, and at least one anti-cancer agent, or a pharmaceutically acceptable salt thereof, for use in treating a cancer in a subject.

19. A method of treating a cancer in a subject in need thereof, the method comprising administering to the subject at least one MetAP2 inhibitor, or a pharmaceutically acceptable salt thereof, and at least one anti-cancer agent, or a pharmaceutically acceptable salt thereof.

20. A MetAP2 inhibitor, or a pharmaceutically acceptable salt thereof, for use in a method of treating a cancer in a subject, wherein the method further comprises administration of at least one anti-cancer agent, or a pharmaceutically acceptable salt thereof.

21. At least one anti-cancer agent, or a pharmaceutically acceptable salt thereof, for use in a method of treating pulmonary fibrosis in a subject, wherein the method further comprises administration of at least one MetAP2 inhibitor or a pharmaceutically acceptable salt thereof.

22. The combination for use of claim 18, the method of claim 19, the MetAP2 inhibitor for use of claim 20, or the at least one anti-cancer agent for use of claim 21, wherein the at least one MetAP2 inhibitor, or pharmaceutically acceptable salt thereof, and the at least one anti-cancer agent, or pharmaceutically acceptable salt thereof, are administered concurrently or in temporal proximity.

23. The combination for use of claim 18 or 22, the method of claim 19 or 22, the MetAP2 inhibitor for use of claim 20 or 22, or the at least one anti-cancer agent for use of claim 21 or 22, wherein the MetAP2 inhibitor is selected from: 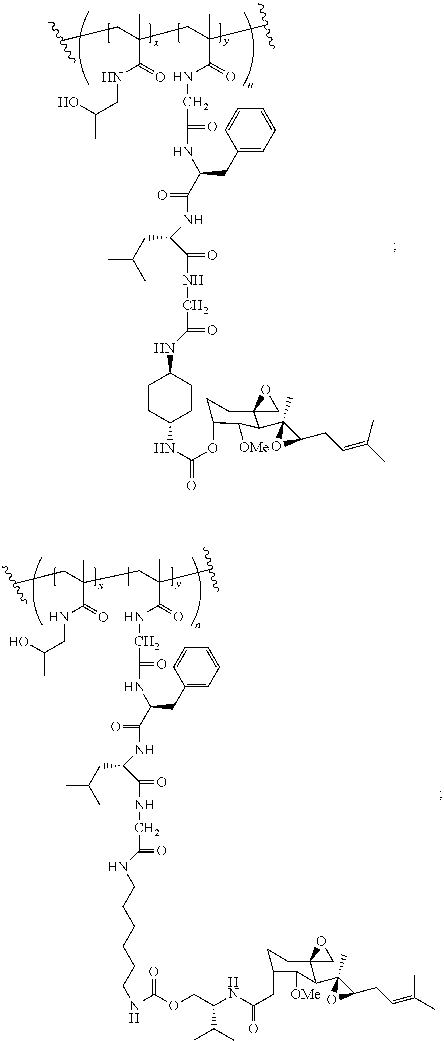 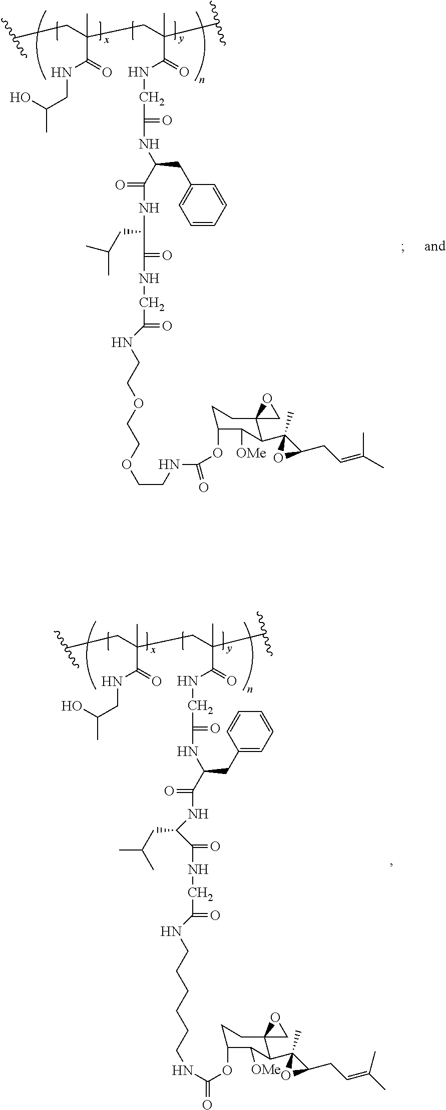
or a pharmaceutically acceptable salt thereof,
wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

24. The combination for use of claim 18 or 22-23, the method of claim 19 or 22-23, the MetAP2 inhibitor for use of claim 20 or 22-23, or the at least one anti-cancer agent for use of claim 21 or 22-23, wherein the MetAP2 inhibitor is: 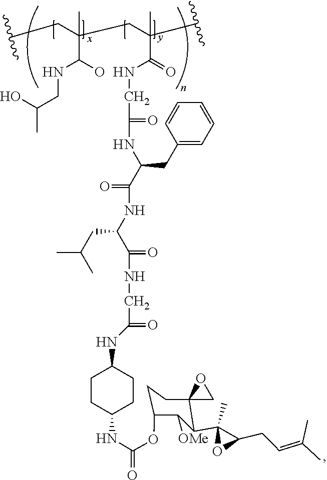
or a pharmaceutically acceptable salt thereof, wherein x is in the range of 1 to about 450, y is in the range of 1 to about 30 and n is in the range of 1 to about 100, preferably wherein the ratio of x to y is in the range of about 30:1 to about 3:1, preferably wherein the ratio of x to y is about 11:1.

25. The combination for use of claim 18 or 22-23, the method of claim 19 or 22-234, the MetAP2 inhibitor for use of claim 20 or 22-24, or the at least one anti-cancer agent for use of claim 21 or 22-24, wherein the at least one anti-cancer agent comprises Enhertu, and wherein the cancer is breast cancer.

26. The combination for use, method, MetAP2 inhibitor for use, or anti-cancer agent for use of claim 25, wherein the breast cancer is HER2-negative breast cancer.

27. The combination for use of claim 1 or 5-8, the method of claim 2 or 5-8, the MetAP2 inhibitor for use of claim 3 or 5-8, or the nintedanib for use of claim 4 or 5-8, wherein the administration of the MetAP2 inhibitor, alone or in combination with nintedanib, results in reduced pulmonary congestion, reduced pulmonary inflammation, reduced respiratory rates, increased SpO₂, increased pO₂ levels, decreased blood lactate levels, increased blood bicarbonate ion levels, decreased lung weight, reduced tissue resistance of the respiratory system, reduced lung tissue damping, reduced elastance of the respiratory system, reduced pulmonary elastance, reduced parenchyma density, reduced pulmonary foci, reduced collagen content, reduced collagen deposition and/or reduced tissue density in the subject.

28. The combination for use of claim 1 or 5-8, the method of claim 2 or 5-8, the MetAP2 inhibitor for use of claim 3 or 5-8, or the nintedanib for use of claim 4 or 5-8, wherein the administration of the MetAP2 inhibitor, alone or in combination with nintedanib, results in a reduction in the deposition of collagen in lung tissue as compared to a subject who was not administered a MetAP2 inhibitor, or a pharmaceutically acceptable salt thereof.

29. The combination for use, method, MetAP2 inhibitor for use, or nintedanib for use of claim 28, wherein the administration of both the MetAP2 inhibitor and nintedanib results in a greater reduction in the deposition of collagen in lung tissue as compared to the administration of the MetAP2 inhibitor, or a pharmaceutically acceptable salt thereof, alone.

30. The combination for use of claim 1 or 5-8, the method of claim 2 or 5-8, the MetAP2 inhibitor for use of claim 3 or 5-8, or the nintedanib for use of claim 4 or 5-8, wherein the pulmonary fibrosis results from an interstitial lung disease.

31. The combination for use, method, MetAP2 inhibitor for use, or nintedanib for use of claim 30, wherein the interstitial lung disease is drug induced.
